(19) United States
(12) Patent Application Publication
(10) Pub. No.: US 2008/0171736 A1
(43) Pub. Date: Jul. 17, 2008

(54) Treatment of Alzheimer's Disease and Mild Cognitive impairment using GnRH-I analogs and one or more of acetylcholinesterase inhibitors and NMDA receptor antagonists

(76) Inventors: Christopher W. Gregory, Cary, NC (US); Patrick S. Smith, Raleigh, NC (US)

Correspondence Address: COVINGTON & BURLING, LLP;ATTN: PATENT DOCKETING, 1201 PENNSYLVANIA AVENUE, N.W., WASHINGTON, DC, 20004-2401, US

(21) Appl. No.: 11/819,699

(22) Filed: Jun. 28, 2007

Related U.S. Application Data
(63) Continuation-in-part of application No. 11/179,608, filed on Jul. 13, 2005, now abandoned.
(60) Provisional application No. 60/638,123, filed on Dec. 23, 2004.

(51) Int. Cl. A61K 31/553; A61K 31/4178; A61K 31/435; A61K 31/445

(52) U.S. Cl. 514/215; 514/397; 514/319; 514/297

(57) ABSTRACT

Methods of treating, mitigating, slowing the progression of, or preventing Alzheimer's Disease and Mild Cognitive Impairment (MCI) include administration of gonadotropin-releasing hormone analogs in combination with acetylcholinesterase inhibitors and/or N-methyl-D-aspartate receptor antagonists.

### RELATED APPLICATIONS

This application is a continuation-in-part of U.S. patent application Ser. No. 11/179,608, filed Jul. 13, 2005, which claims priority under 35 U.S.C. § 119 to U.S. Provisional Patent Application No. 60/638,123, filed Dec. 23, 2004, each of which is incorporated herein by reference in its entirety.

### FIELD OF INVENTION

This invention relates to the treatment, mitigation, slowing the progression of, and prevention of Alzheimer's Disease and Mild Cognitive Impairment (MCI).

### BACKGROUND OF INVENTION

Alzheimer's disease (AD) is a neurodegenerative disorder that leads to progressive memory loss, impairments in behavior, language, and visuo-spatial skills, and ultimately death. The disease is invariably associated with and defined by neuronal and synaptic loss, the presence of extracellular deposits of β-amyloid protein, and intracellular formation of neurofibrillary tangles in the brain (Selkoe D J. Alzheimer disease: Genotypes, phenotypes and treatments. Science 275:630-631, 1997; Smith M A. Alzheimer disease. In: Bradley R J and Harris R A, eds. International Review of Neurobiology, Vol. 42. San Diego, Calif.: Academic Press, Inc. 1-54, 1998). The etiology of AD is not known, although a number of hypotheses exists regarding the mechanisms of damage to the brain. Mild cognitive impairment (MCI) is not a distinct disease condition but rather a form of memory loss that may represent the early stages of AD or may be due to stress or other illness. There is a continuing need for cost-effective approaches for treating, mitigating, slowing the prevention of, and preventing AD and MCI.

### SUMMARY OF INVENTION

Gonadotropin-releasing hormone (GnRH-I) analogs decrease blood and tissue levels of the gonadotropins follicle-stimulating hormone (FSH) and luteinizing hormone (LH). Acetylcholinesterase (AChE) inhibitors increase acetylcholine levels at neuronal synapses, and N-methyl-D-aspartate (NMDA) receptor antagonists decrease glutamate-stimulated excitotoxicity. According to the present invention, GnRH-I analogs in combination with AChE inhibitors and/or NMDA receptor antagonists are effective in treating, mitigating, slowing the progression of, and/or preventing AD and MCI.

In accordance with embodiments of the present invention, decreased blood and tissue levels, production, function, and activity of FSH and LH, along with AChE inhibition at neuronal synapses, prevent aborted cell cycling of terminally differentiated neurons and elevate the levels of acetylcholine in neuronal synapses of the basal forebrain, amygdala, hippocampus, and entorhinal cortex, thus treating, mitigating, slowing the progression of, and/or preventing AD and MCI.

In other embodiments of the invention, decreased blood and tissue levels, production, function, and activity of FSH and LH, along with decreased glutamate-stimulated excitotoxicity, prevent aborted cell cycling of terminally differentiated neurons and prevent neuronal death due to glutamate-induced neuronal excitotoxicity.

In other embodiments of the invention, decreased blood and tissue levels, production, function, and activity of FSH and LH, along with AChE inhibition at neuronal synapses and decreased glutamate-stimulated neuronal excitotoxicity, prevent aborted cell cycling of terminally differentiated neurons, elevate the levels of acetylcholine in neuronal synapses of the basal forebrain, amygdala, hippocampus, and entorhinal cortex, and prevent neuronal death due to glutamate-induced neuronal excitotoxicity.

An embodiment of the present invention provides a method of treating, mitigating, slowing the progression of, or preventing Alzheimer's Disease and MCI, comprising administering a therapeutically effective combination, or a therapeutically effective synergistic combination, of a gonadotropin releasing hormone analog (for example leuprolide acetate, hydrochloride, sulfate, or other salt or the free base), and either or both of an acetylcholinesterase inhibitor (for example donepezil, rivastigimine, galantamine, or tacrine) and an N-methyl-D-aspartate receptor antagonist (for example, memantine). The method of using the combination can comprise the administration of two or more compounds together in the same composition, for example, in one tablet, capsule, implant, injection or nasal spray. The method of using the combination can also comprise the administration of two or more compounds in sequence such that one compound is administered shortly before the administration of the other, or after a delay, e.g., due to differences in dosage regimens of the pharmaceutical materials used.

### BRIEF DESCRIPTION OF THE DRAWINGS

FIG. 1 presents results of a clinical trial comparing administration of a combination of an acetylcholinesterase inhibitor (AChEI) and leuprolide acetate with administration of a combination of an AChEI with placebo, using the Alzheimer's Disease Assessment Scale-Cognitive (ADAS-Cog) test.

FIG. 2 presents results of a clinical trial comparing administration to female subjects of an acetylcholinesterase inhibitor (AChEI) and leuprolide acetate, alone and in combination, using the Alzheimer's Disease Assessment Scale-Cognitive (ADAS-Cog) test.

FIG. 3 presents results of the same clinical trial, using the Alzheimer's Disease Cooperative Study Activities of Daily Living (ADCS-ADL) test.

FIG. 4 presents results of the same clinical trial, using the Alzheimer's Disease Cooperative Study Clinical Global Impression of Change (ADCS-CGIC) test.

FIG. 5 presents results of using leuprolide acetate to inhibit cell growth of neuroblastoma and glioblastoma cell lines according to an embodiment of the invention.

FIG. 6 presents a pharmacokinetic release profile of leuprolide acetate and a serum concentration of testosterone in men receiving ELIGARD® 45 mg.

### DETAILED DESCRIPTION OF EXEMPLARY EMBODIMENTS

### The Gonadotropin Hypothesis of Alzheimer's Disease

The cell cycle hypothesis of AD, which is consistent with known abnormalities associated with the disease, proposes that AD is a result of aberrant re-entry of neurons into the cell cycle. Aberrant cell cycle re-entry has been proposed to be caused by an age-related upregulation of an unknown mitogen. The gonadotropin hypothesis proposes that LH is this mitogen.

LH and human chorionic gonadotropin (HCG) have been shown to be mitogenic in certain reproductive tissues (Horiuchi A. Nikaido T. Yoshizawa T. Itoh K. Kobayashi Y, Toki T, et al. HCG promotes proliferation of uterine leiomyomal cells more strongly than that of myometrial smooth muscle cells in vitro. Molec. Human Reprod. 6:523-528, 2000; Davies B R, Fiimigan D 5, Smith S K, and Ponder B A. Administration of gonadotropins stimulates proliferation of normal mouse ovarian surface epithelium. Gynecol. Endocrinol. 13:75-81, 1999; Webber R J and Sokoloff L. In vitro culture of rabbit growth plate chondrocytes. 1. Age-dependence of response to fibroblast growth factor and “chondrocyte growth factor.” Growth. 45:252-268, 1981).

Further, HCG and LH are frequently expressed by tumor cells (Yokotani T, Koizumi T, Taniguchi R, Nakagawa T, Isobe T, Yoshimura M, et al. Expression of alpha and beta genes of human chorionic gonadotropin in lung cancer. Int. J. Cancer 71:539-544, 1997; Krichevsky A, Campbell-Acevedo E A, Tong J Y, and Acevedo H F. Immunological detection of membrane-associated human luteinizing hormone correlates with gene expression in cultured human cancer and fetal cells. Endocrinol. 136:1034-1039, 1995; Whitfield G K and Kourides I A. Expression of chorionic gonadotropin alpha- and beta-genes in normal and neoplastic human tissues: relationship to deoxyribonucleic acid structure. Endocrinol. 117:231-236, 1985).

In addition, LH has been shown to activate extra-cellular signal-regulated kinase (ERK) and mitogen-activated protein (MAP) kinase. (Srisuparp S, Strakova Z, Brudney A, Mukherjee S, Reierstad S, Hunzicker-Dunn M, et al. Signal transduction pathways activated by chorionic gonadotropin in the primate endometrial epithelial cells. Biol. Reprod. 68:457-464, 2003; Cameron M R, Foster J S, Bukovsky A, and Wimalasena J. Activation of mitogen-activated protein kinases by gonadotropins and cyclic adenosine 5′-monophosphates in porcine granulosa cells. Biol. Reprod. 55:111-119, 1996). Increased serum concentrations of LH also correlate to periods of rapid growth: fetal life, the subsequent first year of life, and puberty. Once reproductive maturity is reached, it is believed that the mitogenicity of LH is countered by newly produced sex steroids and inhibins. However, it is also believed that protection against the mitogenic effects of LH is lost with the age-related decline in reproductive function that results in a decrease in sex steroids and inhibins and an increase in LH. While this hormonal profile may be advantageous in the developing brain of a fetus, terminally differentiated adult neurons are likely to be unable to respond appropriately to mitogenic stimulus, resulting in the neuronal dysfunction and death characteristic of AD.

It has been shown in vitro and in vivo that gonadotropins modulate amyloid-β precursor protein processing and β-amyloid protein generation. (Bowen R L, Verdile G, Liu T, Parlow A F, Perry G, Smith M A, et al. Luteinizing hormone, a reproductive regulator that modulates the processing of amyloid-β precursor protein and amyloid-β deposition. J. Biol. Chem. 279:20539-20545, 2004). In addition, human granulosa cells stimulated with gonadotropins are characterized by upregulation of expression of the presenilin-1 and -2 genes, which code for proteins involved in amyloid-β precursor protein processing. (Rimon E, Sasson R, Dantes A, Land-Bracha A, and Amsterdam A. Gonadotropin-induced gene regulation in human granulosa cells obtained from IVF patients: modulation of genes coding for growth factors and their receptors and genes involved in cancer and other diseases. Int. J. Oncol. 24:1325-1338, 2004).

GnRH-I is widely recognized as the key hormone in the control of reproductive functions. GnRH-I is expressed not only in the hypothalamus but also in peripheral tissues, both normal and in tumors. GnRH-I is generally understood to function by binding to specific receptors of the seven transmembrane family of receptors that are present on the surface of target cells. Binding of the hormone to the receptor activates downstream signaling pathways, and in certain tissues, one of the events triggered by GnRH-I hormone binding to the GnRH-I receptor is the release of gonadotropins into the bloodstream. Activation of the pituitary receptor by binding results in the stimulation of a Gq/11 protein which activates phospholipase C, leading to generation of the second messengers inositol-1,4,5-triphosphate and diacylglycerol, causing mobilization of intracellular pools of calcium and activation of various protein kinase C subspecies. (See Limonta P, Moretti R M, Montagnani M, Motta M. The biology of gonadotropin hormone-releasing hormone: role in the control of tumor growth and progression in humans. Frontiers Neuroendocrinol. 24:279-295, 2003.) GnRH-I also activates phospholipase A2, releasing arachadonic acid and phospholipase D, thereby initiating the production of phosphatidylethanol and phosphatidic acid. In addition, GnRH-I signaling activates mitogen-activated protein kinase (MAPK) cascades, extracellular signal-related kinase (ERK), Jun N-terminal kinase (JNK), p38MAPK, and big MAPK.

GnRH-I has a short half life, and analogs with increased stability have been synthesized as therapeutic agents that have improved pharmacodynamics and pharmacokinetics. By modifying or substituting the glycine residue at position 6 in GnRH-I or deleting glycine 10-amide and adding an ethylamide residue to proline 9, synthetic analogs have been developed with increased half lives and 50-100 times more potency than GnRH-I itself. The GnRH-I peptide chain can also be modified by substituting other synthetic or naturally-occurring amino acid units, including alkylated, acylated or halogenated derivatives of amino acids, or PEGylated amino acid units or PEGylated amino acid derivatives for the amino acid units naturally found in GnRH-I. Generally speaking, when GnRH-I analogs are administered to a patient continuously and at high doses, after an initial upregulation of receptor activation, the GnRH-I receptor is desensitized and downstream signaling is inhibited. Generally, modification of the GnRH-I analog does not prevent the peptide from binding to the GnRH-I receptor to mediate downstream signaling. The GnRH-I receptor is therefore considered to be a “gatekeeper” for GnRH-I analog-mediated signaling. Regardless of the second messenger pathways that are modulated, the initial signal typically originates through the receptor.

In light of the information presented in this specification, GnRH-I analogs useful for practicing the method of the invention may have the same or similar effects on GnRH-I receptors. In some preferred embodiments, a GnRH-I analog useful for the present invention binds selectively to the GnRH-I receptor. In some preferred embodiments, a GnRH-I analog useful for the present invention has an EC₅₀ of between 0.001 pM and 1.0 mM. Further, in embodiments, a GnRH-I analog useful for the present invention may cause at least one of two further processes to occur: 1) the GnRH-I analog may cause desensitization of GnRH-I receptors after an initial activation period of about a day to a week or two, wherein the desensitization or a reduction of receptor numbers leads to inhibition of downstream signaling from the receptors; or 2) the GnRH-I analog may inhibit the normal binding of GnRH-I to its receptor, thereby leading to inhibition of downstream signaling from the receptor. Leuprolide acetate is presented as an example of a GnRH-I analog that can be used to treat Alzheimer's disease, for example, in females, leuprolide acetate and other GnRH-I analogs may also be used to treat Alzheimer's disease and MCI when used in combination with acetylcholinesterase inhibitors and/or NMDA receptor antagonists. However, those of ordinary skill in the art will appreciate that one or more other GnRH-I analogs would be expected to have similar therapeutic effects, due to the commonalities in their mechanisms of action.

### Upregulation of the Cell Cycle

The cell cycle is an orderly sequence of events performed by a cell to replicate itself in which it duplicates its contents and then divides in two. This is the essential mechanism by which all living things reproduce. Humans manufacture millions of new cells every second simply to survive: if all cell division were stopped—by exposure to a very large dose of x-rays, for example—we would die within a few days. The cell cycle occurs in phases, including interphase, which has a gap prior to and after DNA synthesis, and mitosis, during which the nucleus and the cytoplasm divide. Upregulating the cell cycle is achieved through multiple mechanisms, including growth factor stimulation, response to injury, wound repair, and developmental requirements. The cell cycle (and its upregulation) can be analyzed by multiple methods. Cultured cells can be observed under a microscope to count the number of cells that are structurally round rather than firmly adherent to the dish, signifying active proliferation. Other cells can be observed in the process of cytokinesis (division). To identify cells that are synthesizing DNA in preparation for division, cells can be supplied with radioactive H³-thymidine or the artificial thymidine analog bromo-deoxyuridine (BrdU). These nucleotides will be incorporated in the growing DNA strand and nuclei that have incorporated these nucleotides can be identified by autoradiography, in the case of H³-thymidine or visualized with a specific antibody, in the case of BrdU. DNA content doubles during synthesis and DNA-binding fluorescent dyes will identify the DNA content of the cells and can be detected in an instrument known as a flow cytometer.

In Alzheimer's disease, an upregulation of the cell cycling of neurons has been implicated in the pathological changes that occur. Neuronal death has as its root cause the aberrant re-entry of the cells into the process of cell division. It has been proposed that cells may be stuck in a futile cell cycle for up to one year in Alzheimer's brain tissue (Yang Y, Mufson E J, Herrup K. Neuronal cell death is preceded by cell cycle events at all stages of Alzheimer's disease. J. Neurosci. 23:2557-2563, 2003). Cell cycling is incompatible with the differentiated state of the neuron in an adult brain. Neuronal death has been associated with aberrant appearance of cell cycle molecules in Alzheimer's transgenic mouse brains (Andorfer C, Acker C M, Kress Y, H of P R, Duff K, Davies P. Cell-cycle reentry and cell death in transgenic mice expressing nonmutant human tau isoforms. J. Neurosci. 25:5446-5454, 2005). Preventing the upregulation of the cell cycle in terminally differentiated neurons in Alzheimer's brains is desirable as a therapeutic approach. Leuprolide and other GnRH-I analogs are very effective at slowing the growth of neuroblastoma (representing the neuronal phenotype from an Alzheimer's brain) and glioblastoma (representing the glial or supporting cell phenotype from an Alzheimer's brain) cell lines in culture (see below). These cell lines are acceptable and widely utilized models of Alzheimer's disease that can be studied in vitro.

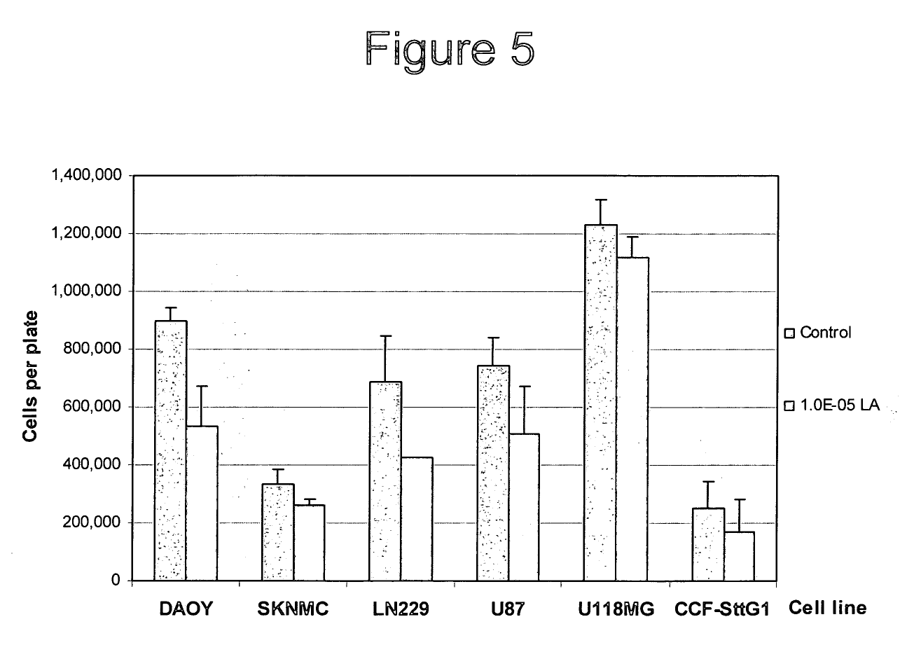

As shown in FIG. 5, leuprolide acetate inhibits cell growth of neuroblastoma cell lines (DAOY (ATCC HTB-186), SKNMC (ATCC HTB-10), and CCF-SttG1 (ATCC CRL-1718)) and glioblastoma cell lines (LN229 (ATCC CRL-2611), U87 (ATCC HTB-14) and U118MG (ATCC HTB-15)). For cell growth assays in a 96 well format, a specific number of cells were plated (about 1000 cells for each line). All cell lines were plated in their respective growth media (supplemented with either 1% regular fetal bovine serum, 1% charcoal/dextran-stripped fetal bovine serum or 0.25% Albumax™ (Invitrogen Corp., Grand Island N.Y.)) and allowed to settle for 24 hours. Leuprolide treatments were commenced immediately after plating the cells. A 10 mM (12.25 mg/ml) solution of leuprolide acetate salt in phosphate buffered saline was prepared and diluted appropriately to obtain the desired final concentrations. Treatment concentrations were 0 M (control) and 10⁻⁵ M (shown as 1.00E-5, 12.25 μg/ml). The number of cells in each group was measured by incubating cells with WST-8 (2-(2-methoxy-4-nitrophenyl)-3-(4-nitrophenyl)-5-(2,4-disulfophenyl)-2H-tetrazolium, monosodium salt) which produces a water soluble formazan dye that was detected by measuring optical density (at 450 nm) using a μQuant™ Universal Microplate Spectrophotometer (Bio-Tek® Instruments, Inc., Winooski, Vt.).

### Therapeutic Strategies Based on the Gonadotropin Hypothesis of AD

According to the present invention, drugs that inhibit gonadotropin synthesis and secretion should result in halting or slowing of the disease process of AD and MCI, and may lead to its mitigation or reversal. A therapeutic strategy for treating AD based on the gonadotropin hypothesis is disclosed in U.S. Pat. No. 6,242,421, issued on Jun. 5, 2001 to Richard L. Bowen, the entirety of which is incorporated herein by reference.

There are a number of drugs approved by the United States Food and Drug Administration (FDA) that effectively suppress gonadotropins. These drugs fall into two classes: GnRH-I agonists (e.g., Zoladex® brand of goserelin acetate) and GnRH-I antagonists (e.g., Plenaxis® brand of abarelix). GnRH-I agonists were developed as a method of suppressing sex steroid production as an alternative to surgical castration in the treatment of advanced prostate cancer. GnRH-I agonists have since been used in a number of other hormone-related conditions, including endometriosis, uterine fibroids, and infertility, and are even approved for use in children suffering from precocious puberty (Filicori M, Hall D A, Loughlin J S, Vale W, and Crowley Jr. W F. A conservative approach to the management of uterine leiomyoma: pituitary desensitization by a luteinizing hormone-releasing hormone analog. Amer. J. Obstetr. Gynecol. 147:726-727, 1983; Laron Z, Kauli R, Zeev Z B, Comaru-Schally A M, and Schally A V. D-TRP5-analog of luteinising hormone releasing hormone in combination with cyproterone acetate to treat precocious puberty. Lancet. 2:955-956, 1981; Meldrum D R, Chang R J, Lu J, Vale W, Rivier J, and Judd H L. “Medical oophorectomy” using a long-acting GNRH agonist-a possible new approach to the treatment of endometriosis. J. Clin. Endocrinol. Metabol. 54:1081-1083, 1982; Wildt L, Diedrich K, van der Ven H, al Hasani S, Hubner H, and Klasen R. Ovarian hyperstimulation for in-vitro fertilization controlled by GnRH-I agonist administered in combination with human menopausal gonadotropins. Human Reprod. 1:15-19, 1986).

For chronic use, GnRH-I agonists are usually more effective than GnRH-I antagonists at suppressing gonadotropins. GnRH-I antagonists were developed to inhibit gonadotropin and sex steroid synthesis and secretion without causing the initial spike or burst in gonadotropins and sex steroids typically associated with GnRH-I agonists. However, while GnRH-I antagonists may prevent this initial burst, there is usually more “breakthrough” in LH and testosterone secretion with use of GnRH-I antagonists than occurs with use of GnRH-I agonists. (Praecis Pharmaceuticals Incorporated, Plenaxis® Package Insert 2004.) This may be due to a compensatory increase in hypothalamic GnRH-I secretion, which alters the ratio of the competing ligands, resulting in activation of the GnRH-I receptor. In contrast, with GnRH-I agonists, a compensatory increase in hypothalamic GnRH-I would only serve to potentiate receptor down-regulation. In addition, GnRH-I antagonists are associated with occasional anaphylactic reactions due to their high histamine releasing properties. (Millar R P, Lu Z L, Pawson A J, Flanagan C A, Morgan K, and Maudsley S R. Gonadotropin-releasing hormone receptors. Endocr. Rev. 25:235-275, 2004).

GnRH-I agonists are analogs of the endogenous GnRH-I decapeptide with specific amino acid substitutions. Replacement of the GnRH-I carboxyl-terminal glycinamide residue with a (short-chain alkyl)-amido-group, such as methylamide group, an ethylamide group or an allylamide group can increase the affinity these analogs possess for the GnRH-I receptor as compared to the endogenous peptide. Many of these analogs also have a longer half-life than endogenous GnRH. Administration of GnRH-I agonists results in an initial increase in serum gonadotropin concentrations that typically persists for several days (there is also a corresponding increase in testosterone in men and estrogen in pre-menopausal women). The initial increase is typically followed by a precipitous decrease in gonadotropins. This suppression is secondary to the loss of GnRH-I signaling due to down-regulation of pituitary GnRH-I receptors (Belchetz P E, Plant T M, Nakai Y, Keogh E J, and Knobil E. Hypophysial responses to continuous and intermittent delivery of hypothalamic gonadotropin-releasing hormone. Science. 202:631-633, 1978). This is believed to be a consequence of the increased concentration of ligand, the increased affinity of the ligand for the receptor, and the continuous receptor exposure to ligand as opposed to the intermittent exposure that occurs with physiological pulsatile secretion of GnRH.

Since GnRH-I agonists are small peptides, they are generally not amenable to oral administration. Therefore, they are customarily administered subcutaneously, intramuscularly, or via nasal spray. GnRH-I agonists are potent, with serum concentrations of less than 1 ng/ml of the GnRH-I agonist leuprolide acetate being considered to be adequate for testosterone suppression. (Fowler J E, Flanagan M, Gleason D M, Klimberg I W, Gottesman J E, and Sharifi R. Evaluation of an implant that delivers leuprolide for 1 year for the palliative treatment of prostate cancer. Urol. 55:639-642, 2000). Due to their small size and high potency, these peptides are strong candidates for use in long-acting depot delivery systems. Many such GnRH-I analog products (as listed below in paragraph 33), each having a duration of action ranging from 1 month to 1 year as depot formulations, are currently marketed in the United States. For the purposes of this invention for the treatment or mitigation or delay of progression or delay of symptoms of Alzheimer's disease and MCI, serum levels should be at or above 0.2 ng/ml, or consistently at above 0.5 ng/mL, or above 0.75 ng/mL, or above 1.0 or 1.5 or 2.0 or 2.5 or 3.0 or 3.5, or 4.0 or higher for extended periods of at least 5 days, at least 10 days, at least 15 days, at least 20 days, at least 30 days, at least 45 days, at least 60 days, at least 90 days, at least 4 months, at least 6 months, at least 9 months, or at least 1 year.

Several of these products comprise leuprolide as the active compound, for example, as found in Lupron Depot®, Viadur™, and ELIGARD®. Zoladex® comprises goserelin as the active ingredient, whereas Synarel® and Suprefact® respectively comprise nafarelin, and buserelin. In addition, both VANTAS® and SUPPRELIN®LA comprise histrelin in inplant form. TRELSTAR® LA and Decapeptyl®SR comprise triptorelin as the active ingredient in injectable, sustained-release form. Several other GnRH-I analogs are also available, including cetrorelix, abarelix, and ganirelix, which are the active ingredients found in Cetrotide®, Plenaxis®, and Antagon™, respectively. Several of these compounds are also formulated for use as sustained-release, injectable agents, or are in formulations amenable to daily administration. These are also known in the art.

Leuprolide has been the active ingredient in products on the market for close to two decades and continues to demonstrate a favorable side effect profile. Most of the side effects such as hot flashes and osteoporosis can be attributed to loss of sex steroid production. (See Stege R. Potential side-effects of endocrine treatment of long duration in prostate cancer. Prostate Suppl. 10:38-42, 2000.) For treatment of female AD patients, sex steroid suppression should not be a major issue since such patients are post-menopausal and their estrogen production is already significantly decreased. However, since males in the same age group normally produce appreciable amounts of testosterone, add-back testosterone supplementation should counter symptoms associated with the suppression of testosterone.

The safety of GnRH-I agonists is further supported by the fact that an estimated well over 100 million doses have been administered to date (based on sales figures) with no serious consistent adverse effects. In addition, the low toxicity of GnRH-I agonists was demonstrated in a clinical trial in which men with prostate cancer received daily injections of leuprolide, for up to two years, that were twenty-fold higher (i.e., 20 mg per day) than the 1 mg per day dose. The 20 mg dose did not result in any adverse effects different from what was seen with the 1 mg dose (TAP Pharmaceuticals, Inc., Lupron Depot® 7.5 mg Package Insert 2003). The safety profile of GnRH-I agonists along with delivery systems that promote compliance for long periods make these compounds well suited for the AD population.

### Exemplary GnRH-I Analogs

Many analogs of GnRH-I are known and have been synthesized. GnRH-I analogs useful for the present invention include the GnRH-I analogs identified in U.S. Pat. No. 5,110,904, the disclosure of which is incorporated herein by reference in its entirety. For instance, GnRH-I analogs contemplated for use in the compositions and methods of the present invention include compounds having a chemical formula represented by the following Chemical Formula I:

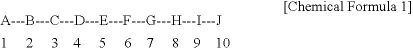

or a pharmaceutically acceptable salt thereof, wherein:

A is an amino acyl residue selected from the group consisting of:
L-pyroglutamyl,
D-pyroglutamyl,
N-acetyl-L-prolyl,
N-acetyl-D-prolyl,
N-acetyl-L-delta-3,4-prolyl,
N-acetyl-D-delta-3,4-prolyl,
N-acetyl-L phenylalanyl,
N-acetyl-D-phenylalanyl,
N-acetyl-L-3-(2-thienyl)alanyl,
N-acetyl-D-3-(2-thienyl)alanyl,
N-acetyl-L-3-(4-chlorophenyl)alanyl,
N-acetyl-D-3-(4-chlorophenyl)alanyl,
N-acetyl-L-3-(4-fluorophenyl)alanyl,
N-acetyl-D-3-(4-fluorophenyl)alanyl,
N-acetyl-L-3-(4-bromophenyl)alanyl,
N-acetyl-D-3-(4-bromophenyl)alanyl,
N-acetyl-L-3-(4-methylphenyl)alanyl,
N-acetyl-D-3-(4-methylphenyl)alanyl,
N-acetyl-L-3-(pentamethylphenyl)alanyl,
N-acetyl-D-3-(pentamethylphenyl)alanyl,
N-acetyl-L-3-(3,4,5-trimethylphenyl)alanyl,
N-acetyl-D-3-(3,4,5-trim ethylphenyl)alanyl,
N-acetyl-L-3-tryptyl(N-indole-methyl),
N-acetyl-D-3-tryptyl(N-indole-methyl),
N-acetyl-L-tryptyl(N-indole-formyl),
N-acetyl-D-tryptyl-(N-indole-formyl),
N-acetyl-L-3-(1-adamantyl)alanyl,
N-acetyl-D-3-(1-adamantyl)alanyl,
N-acetyl-L-5-fluorotryptyl(N-indole-formyl),
N-acetyl-D-5-fluorotryptyl(N-indole-formyl),
N-acetyl-L-3-(2-naphthyl)alanyl,
N-acetyl-D-3-(2-naphthyl)alanyl,
N-acetyl-L-3-(3-benzothienyl)alanyl,
N-acetyl-D-3-(3-benzothienyl)alanyl,
N-acetyl-L-3-(3-benzoxazolyl)alanyl,
N-acetyl-D-3-(3-benzoxazolyl)alanyl,
N-acetyl-alpha-methyl-L-3-(4-chlorophenyl)alanyl,
N-acetyl-alpha-methyl-D-3-(4-chlorophenyl)alanyl,
N-acetyl-L-3-(4-trifluoromethylphenyl)alanyl,
N-acetyl-D-3-(4-trifluoromethylphenyl)alanyl,
N-acetyl-L-tyrosyl,
N-acetyl-D-tyrosyl,
N-acetyl-L-O-methyl-tyrosyl,
N-acetyl-D-O-methyl-tyrosyl,
N-acetyl-D-3-(2-naphthyl)alanyl,
N-acetyl-L-3-(1-naphthyl)alanyl,
N-acetyl-D-3-(1-naphthyl)alanyl,
N-acetylsarcosyl,
N-acetyl-L-3-(cyclohexyl)alanyl,
N-acetyl-D-3-(cyclohexyl)alanyl,
N-acetylglycyl,
L-N-acetyl-N-methylalanyl,
N-acetyl-N-methyl-D-alanyl,
N-acetyl-alpha-methyl-L-phenylalanyl,
N-acetyl-alpha-methyl-D-phenylalanyl,
N-acetyl-D-phenylalanyl,
N-acetyl-L-phenylalanyl,
N-formylsarcosyl,
N-formyl-N-methyl-L-alanyl,
N-formyl-N-methylalanyl,
2-N-beta-(ethylaminocarbonyl)-N-epsilon-(ethylamido)glutamyl,
N-delta-ethyl-glutamyl,
L-prolyl,
D-prolyl,
L-delta-3,4-prolyl,
D-delta-3,4-prolyl,
L-phenylalanyl,
D-phenylalanyl,
L-3-(4-methylphenyl)alanyl),
D-3-(4-methylphenyl)alanyl,
L-3-(4-nitrophenyl)alanyl,
D-3-(4-nitrophenyl)alanyl,
L-3-(4-acetylaminophenyl)alanyl,
D-3-(4-acetylaminophenyl)alanyl,
L-3-(4-chlorophenyl)alanyl,
D-3-(4-chlorophenyl)alanyl,
L-3-(4-fluorophenyl)alanyl,
D-3-(4-fluorophenyl)alanyl,
alpha-methyl-L-3-(4-chlorophenyl)alanyl,
alpha-methyl-D-3-(4-chlorophenyl)alanyl,
L-3-(4-trifluoromethylphenyl)alanyl,
D-3-(4-trifluoromethylphenyl)alanyl,
L-tyrosyl,
D-tyrosyl,
L-O-methyl-tyrosyl,
D-O-methyl-tyrosyl,
sarcosyl, glycyl,
L-N-methylalanyl,
N-methyl-D-alanyl,
N-methyl-L-pyroglutamyl,
N-methyl-D-pyroglutamyl,
alpha-methyl-L-phenylalanyl,
alpha-methyl-D-phenylalanyl,
N-acetyl-alpha-aza-3-(4-chlorophenyl)alanyl,
N-acetyl-alpha-aza-3-(4-fluorophenyl)alanyl,
N-acetyl-alpha-aza-3-(2-naphthyl)alanyl,
N-acetyl-alpha-aza-3-(1-naphthyl)alanyl,
N-acetyl-alpha-aza-alanyl,
N-acetyl-alpha-aza-glycyl,
N-acetyl-alpha-aza-sarcosyl,
N-acetyl-alpha-aza-3-(4-methylphenyl)alanyl,
N-acetyl-alpha-aza-cyclohexylalanyl,
N-acetyl-alpha-aza-3-(1-adamantyl)alanyl,
N-acetyl-alpha-aza-tyrosyl(O-methyl),
N-acetyl-alpha-aza-3-(3-benzothienyl)alanyl,
N-acetyl-alpha-aza-phenylalanyl,
N-methyl-alpha-aza-pyroglutamyl,
N-acetyl-alpha-aza-3-(2-thienyl)alanyl,
N-acetyl-alpha-aza-3-(3-benzoxazolyl)alanyl,
N-acetyl-alpha-aza-3-(3,4,5-trimethylphenyl)alanyl,
N-acetyl-alpha-aza-3-(pentamethylphenyl)alanyl,
N-acetyl-N-alpha-methyl-alpha-aza-3-(2-naphthyl)alanyl,
N-acetyl-N-alpha-methyl-alpha-aza-3-(1-naphthyl)alanyl,
N-acetyl-N-alpha-methyl-alpha-aza-3-(4-chlorophenyl)alanyl,
N-acetyl-N-alpha-methyl-alpha-aza-3-(4-fluorophenyl)alanyl,
N-acetyl-N-alpha-methyl-alpha-aza-3-(4-methylphenyl)alanyl,
N-acetyl-N-alpha-methyl-alpha-aza-3-(4-methoxyphenyl)alanyl,
N-acetyl-N-alpha-methyl-alpha-aza(1-adamantyl)alanyl,
N-acetyl-N-alpha-methyl alpha-aza-3-(phenyl)alanyl,
N-acetyl-N-alpha-methyl-alpha-aza-alanyl,
N-acetyl-N-alpha-methyl alpha-aza-3-(cyclohexyl)alanyl,
N-acetyl-N-alpha-methyl-alpha-aza-3-(benzothienyl)alanyl,
N-acetyl-N-alpha-methyl-alpha-aza-3-(benzoxazolyl)alanyl,
N-acetyl-N-alpha-methyl-alpha-aza-3-(3,4,5-trimethylphenyl)alanyl,
N-acetyl-N-alpha-methyl-alpha-aza-3-(pentamethylphenyl)alanyl and
N-acetyl-N-alpha-methyl-alpha-aza-3-(2-thienyl)alanyl;

B is absent or is an amino acyl residue selected from the group consisting of
L-histidyl,
D-histidyl,
L-tryptyl,
D-tryptyl,
L-tryptyl(N-indole-methyl),
D-tryptyl(N-indole-methyl),
L-phenylalanyl,
D-phenylalanyl,
L-3-(2-naphthyl)-alanyl,
D-3-(2-naphthyl)-alanyl,
L-3-(1-naphthyl)-alanyl,
D-3-(1-naphthyl)-alanyl,
L-3-(3-benzoxazolyl)alanyl,
D-3-(3-benzoxazolyl)alanyl,
L-3-(3-pyridyl)-alanyl,
L-3-(2-pyridyl)-alanyl,
D-3-(3-pyridyl)-alanyl,
D-3-(2-pyridyl)-alanyl,
L-3-(2-thiazolyl)alanyl,
D-3-(2-thiazolyl)-alanyl,
L-3-(3-benzothienyl)alanyl,
D-3-(3-benzothienyl)alanyl,
L-3-(2-benzothienyl)alanyl,
D-3-(2-benzothienyl)alanyl,
L-3-(2-thienyl)-alanyl,
D-3-(2-thienyl)-alanyl,
L-cyclohexylalanyl,
D-cyclohexylalanyl,
L-3-(3-pyrazolyl)alanyl,
D-3-(3-pyrazolyl)alanyl,
L-3-(4-chlorophenyl)alanyl,
D-3-(4-chlorophenyl)alanyl,
L-3-(4-fluorophenyl)alanyl,
D-3-(4-fluorophenyl)alanyl,
L-3-(4-bromophenyl)alanyl,
D-3-(4-bromophenyl)alanyl,
L-3-(4-trifluoromethylphenyl)alanyl,
D-3-(4-trifluoromethylphenyl)alanyl,
L-3-(4-aminophenyl)alanyl,
D-3-(4-aminophenyl)alanyl,
L-3-(4-nitrophenyl)alanyl,
D-3-(4-nitrophenyl)alanyl,
L-3-(4-cyanophenyl)alanyl,
D-3-(4-cyanophenyl)alanyl,
L-tyrosyl-(O-methyl),
D-tyrosyl(O-methyl),
L-3-(4-methylphenyl)alanyl,
D-3-(4-methylphenyl)alanyl,
L-3-(4-nitrophenyl)alanyl,
D-3-(4-nitrophenyl)alanyl,
L-3-(4-acetylaminophenyl)alanyl,
D-3-(4-acetylaminophenyl)alanyl,
L-methionyl, D-methionyl,
L-alpha-methyl-3-(4-chlorophenyl)alanyl,
D-alpha-methyl-3-(4-chlorophenyl)alanyl,
(3S)-1,2,3,4-tetrahydroisoquinoline-3-carbonyl,
(3R)-1,2,3,4-tetrahydroisoquinoline-3-carbonyl,
(2)-N-(ethylaminocarbonyl)-(5)-N-(ethylamido)glutamyl,
alpha-aza-3-(3,4,5-trimethylphenyl)alanyl,
alpha-aza-3-(4-bromophenyl)alanyl,
alpha-aza-3-(4-methylphenyl)alanyl,
alpha-aza-3-(1-naphthyl)alanyl,
alpha-aza-3-(1-adamantyl)alanyl,
L-3-(3-quinolyl)-alanyl,
D-3-(3-quinolyl)-alanyl,
alpha-aza-3-(4-chlorophenyl)alanyl,
alpha-aza-3-(4-fluorophenyl)alanyl,
alpha-aza-3-(2-naphthyl)alanyl,
alpha-aza-3-(3-quinolyl)alanyl,
alpha-aza-phenylalanyl,
alpha-aza-tyrosyl(O-methyl),
alpha-aza-3-(2-thienyl)alanyl,
alpha-aza-3-(3-benzthienyl)alanyl,
alpha-aza-cyclohexylalanyl,
alpha-aza-tryptyl,
alpha-aza-tryptyl(N-indole-methyl),
alpha-aza-tryptyl(N-indole-formyl),
N—(R₃₁)-L-phenylalanyl,
N—(R₃₁)-D-phenylalanyl,
N—(R₃₁)-D-3-(4-chlorophenyl)alanyl,
N—(R₃₁)-L-3-(4-chlorophenyl)alanyl,
N—(R₃₁)-D-3-(4-fluorophenyl)alanyl,
N—(R₃₁)-L-3-(4-fluorophenyl)alanyl,
N—(R₃₁)-L-3-(4-trifluoromethylphenyl)alanyl,
N—(R₃₁)-D-3-(4-trifluoromethylphenyl)alanyl,
N—(R₃₁)-L-3-(cyclohexyl)alanyl,
N—(R₃₁)-D-3-(cyclohexyl)alanyl,
N—(R₃₁)-L-3-(4-bromophenyl)alanyl,
N—(R₃₁)-D-3-(4-bromophenyl)alanyl,
N—(R₃₁)-L-3-(4-nitrophenyl)alanyl,
N—(R₃₁)-D-3-(4-nitrophenyl)alanyl,
L-prolyl, D-prolyl, N—(R₃₁)-L-O-methyltyrosyl,
N—(R₃₁)-L-tyrosyl, N—(R₃₁)-D-O-methyl-tyrosyl,
N—(R₃₁)-D-tyrosyl, N—(R₃₁)-L-histidyl,
N—(R₃₁)-D-histidyl, N—(R₃₁)-L-3-(2-thienyl)alanyl,
N—(R₃₁)-D-3-(2-thienyl)alanyl,
N—(R₃₁)-L-3-(2-thiazolyl)alanyl,
N—(R₃₁)-D-3-(2-thiazolyl)alanyl,
N—(R₃)-L-3-(2-pyridyl)alanyl,
N—(R₃₁)-D-3-(2-pyridyl)alanyl,
N—(R₃₁)-D-3-(2-naphthyl)alanyl,
N—(R₃₁)-L-3-(2-naphthyl)alanyl,
N—(R₃₁)-L-3-(3-benzothienyl)alanyl,
N—(R₃₁)-D-3-(3-benzothienyl)alanyl,
N—(R₃₁)-L-3-(2-benzothienyl)alanyl,
N—(R₃₁)-D-3-(2-benzothienyl)alanyl,
N—(R₃₁)-L-3-(3-benzoxazolyl)alanyl,
N—(R₃₁)-D-3-(3-benzoxazolyl)alanyl,
N—(R₃₁)-L-3-(3-pyridyl)alanyl,
N—(R₃₁)-D-3-(3-pyridyl)alanyl,
N—(R₃)-L-tryptyl, N—(R₃₁)-D-tryptyl,
N—(R₃₁)-L-tryptyl(N-indole-methyl),
N—(R₃₁)-D-tryptyl(N-indole-methyl),
N—(R₃₁)-D-methionyl,
N—(R₃₁)-L-methionyl,
N—(R₃₁)-D-3-(1-naphthyl)alanyl, and
N—(R₃₁)-L-3-(1-naphthyl)alanyl,

wherein R₃₁— is methyl, ethyl, 2-fluoroethyl, 2,2-difluoroethyl, 2,2,2-trifluoroethyl, propyl or isopropyl;

C is an amino acyl residue selected from the group consisting of
L-tryptyl,
D-tryptyl,
L-tryptyl(N-indole-formyl),
D-tryptyl(N-indole-formyl),
L-tryptyl(N-indole-methyl),
D-tryptyl(N-indole-methyl),
5-fluoro-L-tryptyl,
5-fluoro-D-tryptyl,
L-phenylalanyl,
L-prolyl,
D-prolyl,
L-tyrosyl,
D-tyrosyl,
D-phenylalanyl,
D-3-(3-pyridyl)alanyl,
L-3-(3-pyridyl)alanyl,
D-3-(3-pyridyl-N′-oxide)alanyl,
L-3-(3-pyridyl-N′-oxide)alanyl,
D-3-(3-quinolyl)alanyl,
L-3-(3-quinolyl)alanyl,
D-3-(3-quinolyl-N′-oxide)alanyl,
L-3-(3-quinolyl-N′-oxide)alanyl,
D-3-(1-adamantyl)alanyl,
L-3-(1-adamantyl)alanyl,
L-3-(1-naphthyl)alanyl,
D-3-(1-naphthyl)alanyl,
L-3-(3-benzothienyl)alanyl,
D-3-(3-benzothienyl)alanyl,
L-3-(2-benzothienyl)alanyl,
D-3-(2-benzothienyl)alanyl,
L-3-(3-benzoxazolyl)alanyl,
D-3-(3-benzoxazolyl)alanyl,
L-cyclohexylalanyl,
D-cyclohexylalanyl,
L-3-(3-indazolyl)alanyl,
D-3-(3-indazolyl)alanyl,
alpha-methyl-L-phenylalanyl,
alpha-methyl-D-phenylalanyl,
L-3-(2-naphthyl)alanyl,
D-3-(2-naphthyl)alanyl,
L-O-methyltyrosyl,
D-O-methyltyrosyl,
L-3-(4-methylphenyl)alanyl,
D-3-(4-methylphenyl)alanyl,
L-3-(pentamethylphenyl)alanyl,
D-3-(pentamethylphenyl)alanyl,
L-3-(3,4,5-trimethylphenyl)alanyl,
D-3-(3,4,5-trimethylphenyl)alanyl,
L-3-(4-chlorophenyl)alanyl,
D-3-(4-chlorophenyl)alanyl,
alpha-methyl-L-3-(4-chlorophenyl)alanyl,
alpha-methyl-D-3-(4-chlorophenyl)alanyl,
L-3-(4-trifluoromethylphenyl)alanyl,
D-3-(4-trifluoromethylphenyl)alanyl,
L-3-(4-fluorophenyl)alanyl,
D-3-(4-fluorophenyl)alanyl,
L-3-(2-thienyl)-alanyl,
D-3-(2-thienyl)-alanyl,
N—(R₃₂)-L-3-(3-pyridyl)alanyl,
N—(R₃₂)-D-3-(3-pyridyl)alanyl,
N—(R₃₂)-L-3-(3-pyridyl-N′-oxide)alanyl,
N—(R₃₂)-D-3-(3-pyridyl-N′-oxide)alanyl,
L-3-(2-thiazolyl)-alanyl,
D-3-(2-thiazolyl)alanyl,
alpha-aza-3-(1-naphthyl)alanyl,
alpha-aza-tryptyl,
alpha-aza-phenylalanyl,
alpha-aza-3-(2-thienyl)alanyl,
alpha-aza-3-4-methylphenyl)alanyl,
alpha-aza-3-(pentamethylphenyl)alanyl,
alpha-aza-3-(2-naphthyl)alanyl,
alpha-aza-3-(3-benzothienyl)alanyl,
alpha-aza-3-(3-benzoxazolyl)alanyl,
alpha-aza-3-(cyclohexyl)alanyl,
alpha-aza-3-(1-adamantyl)alanyl,
alpha-aza-3-(4-methoxyphenyl)alanyl,
alpha-aza-3-(4-chlorophenyl)alanyl,
alpha-aza-3-(4 bromophenyl)alanyl,
alpha-aza-tryptyl(N-indole-methyl),
alpha-aza-3-(3-pyridyl)alanyl,
alpha-aza-3-(3-quinolyl)alanyl,
alpha-aza-3-(2-thiazolyl)alanyl,
N—(R₃₂)-L-3-(2-thienyl)alanyl,
N—(R₃₂)-D-3-(2-thienyl)alanyl,
L-3-(3-quinolyl)alanyl,
D-3-(3-quinolyl)alanyl,
L-3-(2-naphthyl)alanyl,
D-3-(2-naphthyl)alanyl,
N—(R₃₂)-D-phenylalanyl,
N—(R₃₂)-L-phenylalanyl,
N—(R₃₂)-D-tryptyl,
N—(R₃₂)-L-tryptyl,
N—(R₃₂)-L-tryptyl(N-indole-formyl),
N—(R₃₂)-D-tryptyl(N-indole-formyl),
N—(R₃₂)-L-tryptyl(N-indole-methyl),
N—(R₃₂)-D-tryptyl(N-indole-methyl),
N—(R₃₂)-L-3-(2-thiazolyl)alanyl,
N—(R₃₂)-D-3-(2-thiazolyl)alanyl,
N—(R₃₂)-L-3-(3-pyridyl)alanyl,
N—(R₃₂)-D-3-(3-pyridyl)alanyl,
N—(R₃₂)-D-3-(3-quinolyl)alanyl,
N—(R₃₂)-L-3-(3-quinolyl)alanyl,
N—(R₃₂)-D-3-(1-adamantyl)alanyl,
N—(R₃₂)-L-3-(1-adamantyl)alanyl,
N—(R₃₂)—)-D-3-(4-fluorophenyl)alanyl,
N—(R₃₂)-L-3-(4-fluorophenyl)alanyl,
N—(R₃₂)-D-3-(4-chlorophenyl)alanyl,
N—(R₃₂)-L-3-(4-chlorophenyl)alanyl,
N—(R₃₂)-L-3-(4-trifluoromethylphenyl)alanyl,
N—(R₃₂)-D-3-(4-trifluoromethylphenyl)alanyl,
N—(R₃₂)-D-3-(2-naphthyl)alanyl,
N—(R₃₂)-L-3-(2-naphthyl)alanyl,
N—(R₃₂)-D-3-(1-naphthyl)alanyl,
N—(R₃₂)-L-3-(1-naphthyl)alanyl,
N—(R₃₂)-L-3-(3-benzothienyl)alanyl,
N—(R₃₂)-D-3-(3-benzothienyl)alanyl,
N—(R₃₂)-L-3-(2-benzothienyl)alanyl,
N—(R₃₂)-D-3-(2-benzothienyl)alanyl,
N—(R₃₂)-L-3-(3-benzoxazolyl)alanyl,
N—(R₃₂)-D-3-(3-benzoxazolyl)alanyl,
N—(R₃₂)-L-tyrosyl, N—(R₃₂)-D-tyrosyl,
N—(R₃₂)-L-3-(3,4,5-trimethylphenyl)alanyl,
N—(R₃₂)-D-3-(3,4,5-trimethylphenyl)alanyl,
N—(R₃₂)-L-3-(4-methylphenyl)alanyl,
N—(R₃₂)-D-3-(4-methylphenyl)alanyl,
N—(R₃₂)-L-3-(pentamethylphenyl)alanyl,
N—(R₃₂)-D-3-(pentamethylphenyl)alanyl,
N—(R₃₂)-L-3-(4-bromophenyl)alanyl,
N—(R₃₂)-D-3-(4-bromophenyl)alanyl,
N—(R₃₂)-L-cyclohexylalanyl,
N—(R₃₂)-D-cyclohexylalanyl,
N—(R₃₂)-L-3-(3-indazolyl)alanyl,
N—(R₃₂)-D-3-(3-indazolyl)alanyl,
N-alpha-N—(R₃₂)-alpha-aza-3-(1-naphthyl)alanyl,
N-alpha-(R₃₂)-alpha-aza-3-(3-pyridyl)alanyl,
N-alpha-(R₃₂)-alpha-aza-phenylalanyl,
N-alpha-(R₃₂)-alpha-aza-3-(3-benzothienyl)alanyl,
N-alpha-(R₃₂)-alpha-aza-3-(2-benzothienyl)alanyl,
N-alpha-(R₃₂)-alpha-aza-3-(4-methylphenyl)alanyl,
N-alpha-(R₃₂)-alpha-aza-3-(4-methylphenyl)alanyl,
N-alpha-(R₃₂)-alpha-aza-3-(4-chlorophenyl)alanyl,
N—(R₃₂)—O-methyl-D-tyrosyl and
N—(R₃₂)—O-methyl-L-tyrosyl,

wherein R₃₂ is methyl, ethyl, 2-fluoroethyl, 2,2-difluoroethyl, 2,2,2-trifluoroethyl, propyl or isopropyl;

D is an amino acyl residue selected from the group consisting of
prolyl,
4-hydroxyproline,
L-seryl,
L-seryl(O-benzyl),
L-seryl(O—PO₃H₂),
L-seryl(O—PO₃Me₂),
D-seryl,
D-seryl(O-benzyl),
D-seryl(O—PO₃H₂),
D-seryl(O—PO₃Me₂),
L-glutamine,
D-3-(3-pyridyl)alanyl,
L-3-(3-pyridyl)alanyl,
D-3-(3-pyridyl-N′-oxide)alanyl,
L-3-(3-pyridyl-N′-oxide)alanyl,
D-3-(3-quinolyl)alanyl,
L-3-(3-quinolyl)alanyl,
D-3-(3-quinolyl-N′-oxide)alanyl,
L-3-(3-quinolyl-N′-oxide)alanyl,
L-alpha,beta-diaminopropionyl,
L-alanyl,
L-threonyl,
2,3-diaminopropionyl,
2-amino-3-guanidinopropionyl,
2,3-diaminopropionyl (wherein the 3-amino group is substituted with lower alkyl, 3-pyridinecarbonyl, 2-pyrazinecarbonyl or 2-indolecarbonyl),
N-alpha-aza-glycyl,
N-alpha-aza-alanyl,
N-alpha-(R₀)-alpha-aza-glycyl,
N-alpha-(R₀)-alpha-aza-alanyl,
N-alpha-(R₀)-L-seryl,
N-alpha-(R₀)-L-seryl(O-benzyl),
NN-alpha-(R₀)-L-glutamine,
N-alpha-(R₀)-L-alanyl,
N-alpha-(R₀)-beta-aminopropionyl,
N-alpha-(R₀)—N-beta-ethylaminopropionyl,
N-(R₀)-L-seryl(O—PO₃H₂),
N-(R₀)-L-seryl(O—PO₃Me₂), and
N-(R₀)-L-threonyl, wherein Ro is lower alkyl or allyl;

or D is a glycosyl derivative of D- or L-serine or D- or L-threonine;

or D is a C₁-C₁₂ ether derivative of D- or L-serine or D- or L-threonine;

or D is a polyethyleneglycol ether derivative of D- or L-serine or D- or L-threonine; wherein the ethyleneglycol ether portion is selected from 1, 2, 3, 4 or up to 100 (O—C₂H₄—) units; and is preferably selected from 1, 2, 3, or 4 to 50 (O—C₂H₄—) units; or from 1 to 20 (O—C₂H₄—) units; or from 2 to 20 (O—C₂H₄—) units; or from 3 to 20 (O—C₂H₄—) units; or from 4 to 20 (O—C₂H₄—) units; or from 1 to 10 (O—C₂H₄—) units; or from 2 to 10 (O—C₂H₄—) units; or from 3 to 10 (O—C₂H₄—) units; or from 4 to 10 (O—C₂H₄—) units; and wherein said polyether terminates in a group selected from the group consisting of:
alkyl,
aryl,
—OH,
—OR,
—O(C(O))R,
—O(C(O))NHR,
—O(C(O))NRR,
—O—C₂H₄—CO₂H,
—O—CH₂—CO₂H,
—NH₂,
—NHR,
—NR₂, or
—NH(C(O))R,
—O(C(O))NHR, or
—O(C(O))NRR;

wherein R is selected independently from lower alkyl, cycloalkyl, benzyl or aryl;

E is an amino acyl residue selected from the group consisting of
L-tyrosyl,
L-tyrosyl(O-methyl),
L-tyrosyl(O-ethyl),
L-tyrosyl(O—PO₃H₂),
L-tyrosyl(O—PO₃Me₂),
D-alanyl,
L-phenylalanyl,
N—(R₃₃)-L-tyrosyl,
N—(R₃₃)-L-tyrosyl(O-methyl),
N—(R₃₃)-L-tyrosyl(O—PO₃H₂),
N—(R₃₃)-L-tyrosyl(O—PO₃Me₂),
3-(2-thienyl)alanyl,
3-(3-benzothienyl)alanyl,
3-(1-naphthyl)alanyl,
3-(2-naphthyl)alanyl,
N—(R₃₃)-L-phenylalanyl,
L-3-(4-chlorophenyl)alanyl,
L-3-(4-fluorophenyl)alanyl,
L-histidyl,
L-3-(cyclohexyl)alanyl,
L-3-(4-aminophenyl)alanyl,
1-3-(4 acetylaminophenyl)alanyl,
N—(R₃₃)-L-3-(4-aminophenyl)alanyl,
N—(R₃₃)-L-3-(4-acetylaminophenyl)alanyl,
N—(R₃₃)-L-3-(4-fluorophenyl)alanyl,
N—(R₃₃)-L-3-(4-chlorophenyl)alanyl,
N—(R₃₃)-L-histidyl,
N—(R₃₃)-L-3-(cyclohexyl)alanyl,
N—(R₃₃)-3-(2-thienyl)alanyl,
N—(R₃₃)-3-(3-benzothienyl)alanyl,
N—(R₃₃)-3-(1-naphthyl)alanyl,
N—(R₃₃)-3-(2-naphthyl)alanyl, and
N—(R₃₃)-L-tyrosyl(O-ethyl),

wherein (R₃₃) is methyl, ethyl, 2-fluoroethyl, 2,2-difluoroethyl, 2,2,2-trifluoroethyl, propyl or isopropyl;

or E is

—N(R₃₀)—C((CH₂)nR₁)H—(CO)—

wherein n is 1 to 5; R₃₀ is hydrogen, methyl, ethyl, propyl or isopropyl; and R₁ is amino, alkylamino, cycloalkylamino or alkanoylamino; or R₁ is —N(R₃)—C(O)(CH₂)_{ff}R₆₀ or —NHC(N(R₃)₂)═NR₄ wherein R₃ is hydrogen, lower alkyl or cycloalkyl; R₄ is hydrogen, lower alkyl, cycloalkyl, amino or cyano; ff is 0 to 6; and R₆₀ is loweralkyl, dialkylamino, cycloalkyl, aryl, arylalkyl, heterocyclic, (heterocyclic)alkyl or —NHR₁₂₀ wherein R₁₂₀ is hydrogen, loweralkyl, cycloalkyl, aryl, arylalkyl, heterocyclic, (heterocyclic)alkyl, amino, alkanoylamino or —NHR₆₂ wherein R₆₂ is loweralkyl, cycloalkyl, aryl, arylalkyl, heterocyclic, (heterocyclic)alkyl or —C(O)R₆₃ wherein R₆₃ is loweralkyl, cycloalkyl, aryl, arylalkyl, heterocyclic or (heterocyclic)alkyl;

or R₁ is —C(O)R** wherein R** is hydroxy, alkoxy, amino, phenoxy or -methoxyphenyl;

F is a D-aminoacyl residue derived from any of the naturally-occurring alpha amino acids, or from synthetic, non-natural alpha amino acids including (t-Bu)-D-glycyl-, D-seryl(O-tBu), D-3-(2-naphthyl)alanyl, Nt-benzyl-D-histidyl-, and including but not limited to those listed for group B; or

F is a D-amino acyl residue having the formula:

—N(R₃₄)—C((CH₂)_{z}(CO)R₃₇)H—(CO)—

wherein z is 0 to 3 and R₃₇ is hydroxy, alkoxy, phenoxy, amino or p-methoxyphenyl and R₃₄ is hydrogen, methyl, ethyl, propyl or isopropyl;

or F is a D-lysine residue or D-homolysine residue substituted with a polyethyleneglycol group (PEG) on the distal nitrogen wherein the PEG unit is attached via acylation to give an amide derivative of a structure selected from the group consisting of:

—NH—C((CH₂)y-NH—(CO)(CH₂)x-(O—C₂H₄—)n-OH))H—(CO)—; or

—NH—C((CH₂)y-NH—(CO)(CH₂))x-(O—C₂H₄—)n-OR))H—(CO)—;

wherein y is 4 or 5; x is an integer selected from 1-10 inclusive; n is an integer selected from 1-100 inclusive; preferably selected from 1-50, more preferably selected from 1-20; and R is a group selected from lower alkyl, aryl, heteroaryl, benzyl, acyl, aroyl, or heteroaroyl; or

or F is a D-lysine residue or D-homolysine residue substituted with a polyethyleneglycol group (PEG) on the distal nitrogen wherein the PEG unit is attached via alkylation to give an amine derivative of a structure such as:

—NH—C((CH₂)y-NH—(CH₂)xCH₂—(O—C₂H₄—)n-OR))H—(CO)—;

wherein y is 4 or 5; x is an integer selected from 1-10 inclusive; n is an integer from 1-100 inclusive; preferably selected from 1-50, more preferably selected from 1-20; and R is a group selected from lower alkyl, aryl, heteroaryl, benzyl, acyl, aroyl, or heteroaroyl;

or F is a
D-citrullinyl residue,
D-ornithinyl residue,
D-lysyl residue or
D-homolysyl residue; or

is a D-ornithinyl residue, D-lysyl residue or D-homolysyl residue substituted on the distal nitrogen with one or two groups selected independently from the group consisting of:
H,
lower alkyl,
aryl,
heteroaryl,
cycloalkyl,
ureido,
guanidinyl,
—(C(O))R,
—(C(O))NHR,
—(C(O))NRR,
C₂H₄—CO₂H,
—CH₂—CO₂H,
—NH₂,
—NHR,
—NR₂,
—NH(C(O))R,
(C(O))NHR, and
—(C(O))NRR; and

wherein R is selected independently from lower alkyl, cycloalkyl, benzyl or aryl;

or F is a glycosyl derivative of D- or L-serine or D- or L-threonine;

or F is a polyethyleneglycol ether derivative of D- or L-serine or D- or L-threonine; wherein the ethyleneglycol ether portion is

selected from the group consisting of from 1-100 (O—C₂H₄—) units; or

alternatively is selected from 1-50 (O—C₂H₄—) units; or

alternatively is selected from 1 to 20 (O—C₂H₄—) units; or

alternatively is selected from 2 to 20 (O—C₂H₄—) units; or from 3 to 20 (O—C₂H₄—) units; or from 4 to 20 (O—C₂H₄—) units; or from 1 to 10 (O—C₂H₄—) units; or from 2 to 10 (O—C₂H₄—) units; or from 3 to 10 (O—C₂H₄—) units; or from 4 to 10 (O—C₂H₄—) units; and

wherein said polyether unit terminates in a group selected from the group consisting of:
alkyl,
aryl,
—OH,
—OR,
O(C(O))R,
—O(C(O))NHR,
—O(C(O))NRR,
—O—C₂H₄—CO₂H,
—O—CH₂—CO₂H,
—NH₂, —NHR,
—NR₂,
—NH(C(O))R,
—O(C(O))NHR, and
—O(C(O))NRR; and

wherein R is selected independently from lower alkyl, cycloalkyl, benzyl or aryl;

provided that if D is a glycosyl derivative or a polyethyleneglycol ether derivative of a serine or threonine, then F can not be a glycosyl derivative or a polyethyleneglycol ether derivative of a serine or threonine or lysine;

G is an amino acyl residue selected from the group consisting of
L-arginyl,
L-leucyl,
D-leucyl,
(t-Bu)-D-glycyl-,
(t-Bu)-L-glycyl-,
L-isoleucyl,
norleucyl,
alloisoleucyl,
valyl,
norvalyl,
seryl(O-t-Bu),
tyrosyl,
tryptyl,
2-aminobutyryl,
L-(cyclohexyl)alanyl,
phenylalanyl,
D-tryptyl,
tyrosyl,
seryl(O-alkyl),
prolyl, pipecolyl,
L-(β-nicotinoyl)lysyl,
seryl and D-seryl;

H is an amino acyl residue selected from
L-prolyl,
L-arginyl,
L-leucyl,
L-(β-nicotinoyl)lysyl; or

H is of the formula:

—N(R₃₀)—C((CH₂)nR₁)H—(CO)—

wherein n is 1 to 5; R₃₀ is hydrogen, methyl, ethyl, propyl or isopropyl; and R₁ is amino, alkylamino, cycloalkylamino or alkanoylamino; or R₁ is —N(R₃)—C(O)(CH₂)_{ff}R₆₀ or —NHC(N(R₃)₂)═NR₄ wherein R₃ is hydrogen, lower alkyl or cycloalkyl; R₄ is hydrogen, lower alkyl, cycloalkyl, amino or cyano; ff is 0 to 6; and R₆₀ is loweralkyl, dialkylamino, cycloalkyl, aryl, arylalkyl, heterocyclic, (heterocyclic)alkyl or —NHR₁₂₀ wherein R₁₂₀ is hydrogen, loweralkyl, cycloalkyl, aryl, arylalkyl, heterocyclic, (heterocyclic)alkyl, amino, alkanoylamino or —NHR₆₂ wherein R₆₂ is loweralkyl, cycloalkyl, aryl, arylalkyl, heterocyclic, (heterocyclic)alkyl or —C(O)R₆₃ wherein R₆₃ is loweralkyl, cycloalkyl, aryl, arylalkyl, heterocyclic or (heterocyclic)alkyl;

I is an NH₂ or NHR group wherein R is selected from lower alkyl such as
methyl,
ethyl,
propyl,
hydroxyethyl,
fluoroethyl,
difluoroethyl, or
trifluoroethyl; or

I is imino acyl or aliphatic amino acyl residue selected from the group consisting of
L-prolyl,
L-pipecolyl,
alpha-aza-prolyl,
trans-beta-aminocyclopentanecarbonyl,
cis-beta-aminocyclopentanecarbonyl,
3-(lower alkyl)-prolyl,
N-methyl-L-alanyl,
N-methyl-norvalyl,
1-dihydroisoindole-2-L-carbonyl and
thiazolidine-5-L-carbonyl; or

I is L-(β-isopropyl)lysyl;

and J is nothing if I is an NH₂ or NHR group; or J is
1-pyrrolidinyl,
1-piperidinyl,
4-morpholinyl, or

an amino acyl residue selected from
D-alanylamide,
L-alanylamide,
glycylamide,
sarcosylamide,
N—(R₄₀)-D-alanylamide,
N—(R₄₀)-L-alanylamide,
N—(R₄₀)-beta-L-alanylamide,
N—(R₄₀)-beta-D-alanylamide,
L-2-aminobutyrylamide,
D-2-aminobutyrylamide,
N—(R₄₀)-L-2-aminobutyrylamide,
N—(R₄₀)-D-2-aminobutyrylamide,
L-serylamide,
D-serylamide,
N—(R₄₀)-L-serylamide,
N—(R₄₀)-D-serylamide,
N—(R₄₀)-L-norvalylamide,
N—(R₄₀)-D-norvalylamide,
L-norvalylamide,
D-norvalylamide,
alpha-aza-glycylamide or
alpha-aza-alanylamide,
   wherein R₄₀ is H, methyl, ethyl, propyl or isopropyl.

As set forth above, and for convenience in describing this invention, the conventional abbreviations for the various common amino acids are used as generally accepted in the peptide art as recommended by the IUPAC-IUB Commission on Biochemical Nomenclature, Biochemistry II, 1726-1972). These represent L-amino acids, with the exception of the achiral amino acid glycine, and with the further exception of any unnatural or natural amino acids which are achiral, or are otherwise designated as D-. The peptide sequences mentioned herein are written according to the generally accepted convention whereby the N-terminal amino acid is on the left and the C-terminal amino acid is on the right.

As used herein, the term “pharmaceutically acceptable salts” refers to salts that retain the desired biological activity of the parent compound and do not impart any undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, trifluoroacetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, methanesulfonic acid, p-toluenesulfonic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or with an organic cation formed from N,N′-dibenzylethylene-diamine or ethylenediamine; or (c) combinations, of (a) and (b), e.g., a zinc tannate salt and the like.

The term “loweralkyl” refers to a straight or branched chain saturated hydrocarbon group having from 1 to 6 carbon atoms such as, for example, methyl, ethyl, n-propyl, isopropyl, n-butyl, isobutyl, sec-butyl, tert-butyl, n-pentyl and n-hexyl.

The term “alkyl of 1 to 12 carbon atoms” refers to a straight or branched chain radical of 1 to 12 carbon atoms.

The term “cycloalkyl” refers to a cyclic saturated hydrocarbon group having from 3 to 7 carbon atoms, for example, cyclopropyl, cyclobutyl, cyclopentyl, cyclohexyl and cycloheptyl.

The term “alkoxy” refers to —OR₄₁ wherein R₄₁ is lower alkyl including, but not limited to, methoxy, ethoxy, t-butyloxy and the like.

The term “thioalkoxy” refers to —SR₄₂ wherein R₄₂ is loweralkyl including, but not limited to, —SCH₃, —SEt and the like.

The term “alkylamino” refers to —NHR₄₄ wherein R₄₄ is loweralkyl including, but not limited to, methylamino, ethylamino and the like.

The term “dialkylamino” refers to —NR₄₅R₄₆ wherein R₄₅ and R₄₆ are independently selected from loweralkyl including, but not limited to, dimethylamino, N-methyl-N-ethyl-amino and the like.

The term “cycloalkylamino” as used herein refers to —NHR₁₃₀ wherein R₁₃₀ is a cycloalkyl group.

The term “halogen” or “halo” as used herein refers to I, Br, Cl or F.

The term “alkanoyl” as used herein refers to —C(O)R₁₃₁ wherein R₁₃₀ is loweralkyl.

The term “alkanoylamino” as used herein refers to R₉₀C(O)NH— wherein R₉₀ is loweralkyl.

The term “alkoxycarbonyl” as used herein refers to R₉₁OC(O)— wherein R₉₁ is loweralkyl.

The term “aryl” as used herein refers to a monocyclic or bicyclic carbocyclic ring system comprising an aromatic carbocyclic ring. Aryl groups include, but are not limited to, phenyl, naphthyl, indanyl, indenyl, tetrahydronaphthyl and the like. Aryl groups can be unsubstituted or substituted with one, two or three substituents independently selected from halogen, loweralkyl, hydroxy, alkoxy, thioalkoxy, nitro, cyano, amino, alkylamino, dialkylamino, alkanoylamino, trihalomethyl and alkoxycarbonyl. Where a specific aryl group is mentioned as a substituent in a compound of this invention, it is to be understood that this invention is intended to encompass compounds comprising any aryl group in place of the specific aryl groups mentioned. In particular, where a specifically substituted phenyl group is mentioned as a substituent in a compound of this invention, it is to be understood that this invention is intended to encompass phenyl groups with other substituents selected from the list given above in place of the specific substituent(s) mentioned.

The term “arylalkyl” as used herein refers to an aryl group appended to a loweralkyl radical including, but limited to, benzyl, naphthylmethyl, 4-methoxybenzyl and the like.

The term “heterocyclic” or “heterocyclic group” as used herein refers to any 3-, 4-, 5- or 6-membered ring containing a heteroatom selected from oxygen, sulfur and nitrogen, or a 5- or 6-membered ring containing one, two or three nitrogen atoms; one nitrogen and one sulfur atom; or one nitrogen and one oxygen atom; wherein the nitrogen and sulfur heteroatoms can optionally be oxidized; wherein the nitrogen heteroatoms can optionally be quaternized; and wherein the 5-membered ring has 0-2 double bonds and the 6-membered ring has 0-3 double bonds. Heterocyclics also include any bicyclic group in which any of the above heterocyclic rings is fused to a benzene ring or another 5- or 6-membered heterocyclic ring independently defined as above. Heterocyclics include, but are not limited to, guinolyl, indolyl, benzofuryl, benzothienyl, imidazolyl, thiazolyl, benzoxazolyl, furyl, thienyl, pyridyl, pyrimidinyl, morpholinyl, piperazinyl, pyrrolidinyl, piperidinyl, thienyl, pyrazinyl, pyrazolyl, thiomorpholinyl, isoquinolyl, indazolyl and the like. Where a specific heterocyclic group is mentioned as a substituent in a compound of this invention, it is to be understood that this invention is intended to encompass compounds comprising any heterocyclic group in place of the specific heterocyclic group(s) mentioned.

Heterocyclics can be unsubstituted or substituted with substituents selected from hydroxy, halo, amino, alkylamino, dialkylamino, alkoxy, thioalkoxy, formyl, alkanoyl, alkanoylamino, benzyl, loweralkyl, cycloalkyl and trihaloalkyl.

The term “(heterocyclic)alkyl” as used herein refers to a heterocyclic group appended to a loweralkyl radical.

The term “glycosyl derivative of serine or threonine” as used herein refers to a serine or threonine residue which is bonded through its hydroxyl group (either alpha- or beta-glycosidically) to a glycosyl radical. Glycosyl radical are derived from a glycopyranose, glycofuranose or an oligosaccharide (all of which can be optionally protected). These glycosyl radicals are derived from D- or L-monosaccharides such as ribose, arabinose, xylose, lyxose, allose, altrose, glucose, mannose, gulose, idose, galactose, talose, erythrose, threose, psicose, fructose, sorbose, tagatose, xylulose, fucose, rhamnose, olivose, oliose, mycarose, rhodosamine, N-acetylglucosamine, N-acetylgalactosamine, N-acetylmannosamine; or disaccharides such as maltose, lactose, cellobiose, gentibiose, N-acetyllactosamine, chitobiose, beta-galactopyranosyl-(1,3)-N-acetylgalactosamine and beta-galactopyranosyl-(1,3)- or (1,4)-N-acetylglucosamine, as well as their synthetic derivatives, such as 2-deoxy, 2-amino, 2-acetamideo- or 2-halogeno derivatives.

### The Cholinergic Hypothesis of Alzheimer's Disease

The cholinergic hypothesis of AD proposes that cholinergic neurons in the basal forebrain degenerate, leading to decreased cholinergic neurotransmission in the cerebral cortex. These changes are thought to contribute to the learning and memory deficits associated with AD.

The enzyme acetylcholinesterase (AChE) hydrolyzes acetylcholine, thereby making it a suitable substrate for binding to the acetylcholine muscarinic and nicotinic receptors, which activate downstream signaling pathways in the cortical pyramidal neurons. In brains with AD, there is an alteration in neurotransmission resulting from reduced levels of acetylcholine. AChE breaks down the acetylcholine that is produced, thereby decreasing activation of postsynaptic acetylcholine muscarinic and nicotinic receptors, which is believed to result in decreased processing of amyloid precursor protein, increased amyloid-β production, and accumulation of hyperphosphorylated tau protein, all hallmarks of AD pathology. Inhibition of AChE enzyme activity is believed to reduce the breakdown of endogenously released acetylcholine, which is expected to result in increased activation of postsynaptic receptors with the end result of reversing the deleterious consequences described above.

### Therapeutic Strategies Based on the Cholinergic Hypothesis

Four AChE inhibitors are currently marketed to improve central cholinergic neurotransmission and are used to treat AD due to their positive effects on memory and cognitive impairment (Racchi M, Mazzucchelli M, Porrello E, Lanni C, Govoni S. Acetylcholinesterase inhibitors: novel activities of old molecules. Pharmacol. Res. 50:441-451, 2004). Donepezil (marketed under the name Aricept®) is a piperidine-based, reversible AChE inhibitor that is highly selective for AChE. Rivastigmine (marketed under the name Exelon®) is a carbamylating, pseudo-irreversible AChE inhibitor that shows dose-dependent cognitive and behavioral benefits in mild-to-moderate AD patients. Galantamine (marketed under the name Reminyl® or Razadyne®), a tertiary alkaloid, is a reversible, competitive AChE inhibitor that has been shown to produce beneficial effects on cognition and the ability to perform activities of daily living. Tetrahydroaminoacridine (tacrine) (marketed under the name Cognex®), was the first acetylcholinesterase inhibitor approved for use in Alzheimer's patients. These compounds are available for the symptomatic treatment of patients with mild-to-moderate AD and are considered to be effective for short-term intervention. While the primary efficacy of this family of compounds likely results from the prevention of acetylcholine breakdown, recent work suggests that these drugs may also interfere with the amyloid cascade by preventing accumulation of amyloid-β. (See Giacobii E. Cholinesterase inhibitors stabilize Alzheimer disease. Neurochem. Res. 25:1185-1190, 2000)

### The Neuronal Glutamate Hypothesis of AD

Neuronal excitotoxicity resulting from glutamate overstimulation of the N-methyl-D-aspartate (NMDA) receptor may play a role in AD pathophysiology. Activation of the NMDA receptor is critical for normal cognitive function (Shimizu E, Tang Y P, Rampon C, Tsien J Z. (2000) NMDA receptor-dependent synaptic reinforcement as a crucial process for memory consolidation [published correction in Science 2001, 291:1902]. Science 290:1170-1174, 2000). Overstimulation of the receptor by glutamate causes increased intracellular calcium and is implicated in neuronal death.

### Therapeutic Strategy Based on the Neuronal Glutamate Hypothesis

Memantine (marketed under the name NAMENDA® and Ebixa®), a noncompetitive antagonist with moderate affinity for the NMDA receptor, blocks neuronal toxicity caused by glutamate. Memantine is approved for use in treating moderate to severe AD.

### Combination Therapy for AD

Each of GnRH-I analogs, AChE inhibitors, and NMDA receptor antagonists, when used separately, has a distinct mechanism of action. Treatment of mild to moderate AD patients with leuprolide acetate as the salt form of the active agent leuprolide, for example, typically prevents the aberrant re-entry of terminal neurons into the cell cycle, thereby preventing neuronal cell death characteristic of AD brains. AChE inhibitors typically improve cholinergic neurotransmission in viable neurons. NMDA receptor antagonists typically prevent glutamate-induced neuronal toxicity. Concomitant use of memantine typically does not inhibit the action of acetylcholinesterase inhibitors.

According to the present invention, combined use of one or more GnRH-I analogs such as leuprolide, goserelin, triptorelin, nafarelin, histrelin, buserelin, cetrorelix, abarelix, or ganirelix (comprising the free-base or acetate, alkyl carboxylate, benzoate, aryl carboxylate, pamoate, hydrochloride, hydrobromide, sulfate, oxalate, mesylate, or other salt forms including polymer-bound anionic salt forms) with the use of one or more AChE inhibitors such as donepezil, rivastigmine, galantamine, or tacrine (comprising the free-base or acetate, pamoate, hydrochloride, hydrobromide, sulfate, oxalate, mesylate, or other salt forms including polymer-bound anionic salt forms) prevents or slows neuronal cell death and improves neurotransmission in surviving cells, resulting in very beneficial results to patients treated with such combinations.

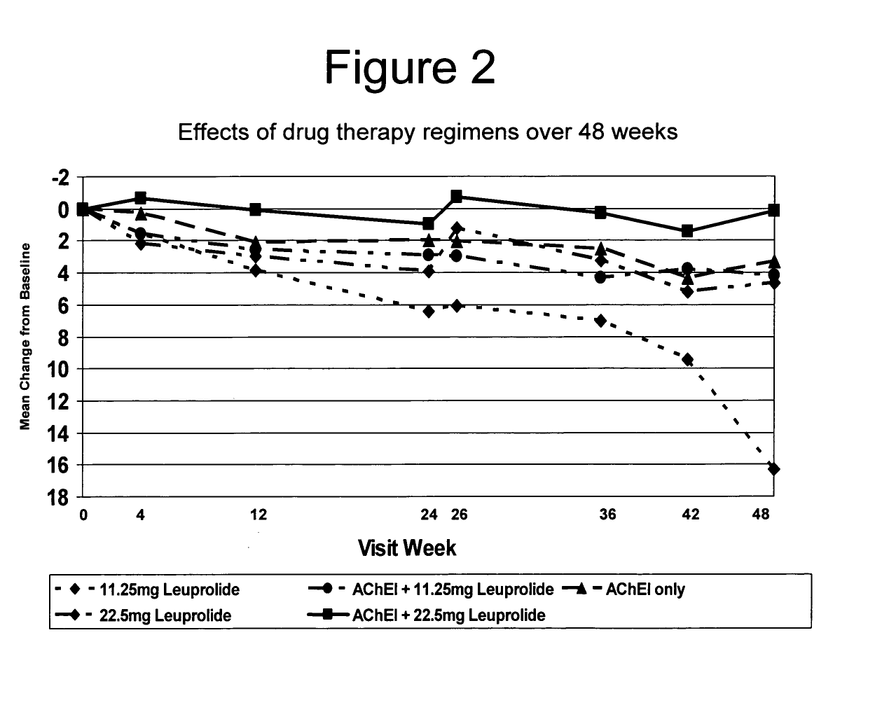

After as little as two months of therapy, combined therapy regimens of GnRH-I analog plus AChEI can yield benefits to the cognitive functioning in patients with Alzheimer's disease or mild cognitive impairment (MCI) over those benefits from using GnRH-I analog alone, AChE inhibitor alone, or no treatment. Similarly, after 4 months, 6 months, 8 months, 10 months, and a year of combination therapy of GnRH-I analog and AChEI, scores on cognition tests and on tests measuring patients' ability in daily activities are much closer to the patients' baseline entry values than the scores of those patients on monotherapy or patients who are not treated with these drugs. This is especially apparent in women who are on a combination therapy regimen of a GnRH-I analog and an AChE inhibitor, who for unexpected reasons show very beneficial, synergistic outcomes on their scores in general at various time periods (see FIG. 2). The prevention of cognitive decline, or slowing of the rate of decline of cognitive functioning and slowing the rate of decline of the mental abilities of patients to take care of basic personal tasks such as brushing their own teeth or using the toilet, is much more apparent in patients on combined GnRH-I analog/AChEI treatment than in those patients in other groups at corresponding time periods.

For example, as shown in FIG. 2, females receiving AChEIs only declined 3.3 points over 48 weeks on the ADAS-cog outcome measure. Females receiving 11.25 mg Lupron Depot® alone declined 16 points over 48 weeks, whereas females receiving 22.5 mg Lupron Depot® declined 4.5 points over 48 weeks. When females were administered 11.25 mg Lupron Depot® together with AChEIs, they declined about 4 points on the ADAS-cog scale while those receiving 22.5 mg Lupron Depot® and AChEIs did not worsen, but instead were stable at the end of 48 weeks.

According to Lon S. Schneider, M D, an expert clinician who is involved in the design and conduct of Alzheimer's disease trials and who is the chairman of Voyager Pharmaceutical Corporation's Medical and Scientific Advisory Board, the expected decline on the ADAS-cog scale for people with mild-to-moderate Alzheimer's disease who are receiving AChEIs is 1.9 points over 6 months, 4 points over 12 months, and 6.4 points over 18 months. In an ongoing trial of tramiprosate in Alzheimer's disease patients, the overall 18 month decline on the ADAS-cog in mild patients was 5.5 points and the overall 18 month decline in moderate patients was 6.3 points.

The examples disclosed in the following tables illustrate exemplary combination therapies that may be implemented by practitioners according to the present invention. As shown in Tables A-D, combination therapies for treating AD may comprise administering to patients various combinations of GnRH-I analogs and AChE inhibitors, such as Lupron Depot®, Viadur®, ELIGARD®, Zoladex®, Synarel®, TRELSTAR®, SUPPRELIN, VANTAS®, Suprefact®, Cetrotide®, Plenaxis®, Antagon™, Decapeptyl® SR, Aricept®, donepezil, rivastigmine, galantamine, and tacrine. Those of ordinary skill will appreciate that variations of the dosage regimens disclosed in Tables A-D may also be used according to various embodiments of the invention. For instance, those of ordinary skill in the art will appreciate that treatment regimens, consistent with prescribing guidelines, may be varied according to the severity of AD in a patient.

TABLE A
Combination of Donepezil (Aricept ®) with selected GnRH-I analog products
for use in a method according to an embodiment of the invention.

| | Dosage Regimen | |
|---|---|---|
| | AChE inhibitor | |
| | Donepezil | Donepezil |
| Dosage Regimen | (Aricept ® 5 mg tablet once | (Aricept ® 10 mg tablet |
| GnRH-I Analogs | per day) | once per day) |
| Lupron Depot ® 3.75 mg 1 | Lupron Depot ® 3.75 mg 1 | Lupron Depot ® 3.75 mg 1 |
| month injection | month injection + Aricept ® | month injection + Aricept ® |
| | 5 mg Tab | 10 mg Tab |
| Lupron Depot ® 7.5 mg 1 | Lupron Depot ® 7.5 mg 1 | Lupron Depot ® 7.5 mg 1 |
| month injection | month injection + Aricept ® | month injection + Aricept ® |
| | 5 mg Tab | 10 mg Tab |
| Lupron Depot-PED ® | Lupron Depot-PED ® | Lupron Depot-PED ® |
| 11.25 mg 1 month injection | 11.25 mg 1 month injection + Aricept ® | 11.25 mg 1 month injection + Aricept ® |
| | 5 mg Tab | 10 mg Tab |
| Lupron Depot-PED ® 15 mg | Lupron Depot-PED ® | Lupron Depot-PED ® |
| injection | 15 mg injection + Aricept ® | 15 mg injection + Aricept ® |
| | 5 mg Tab | 10 mg Tab |
| Lupron Depot ® 22.5 mg 3 | Lupron Depot ® 22.5 mg 3 | Lupron Depot ® 22.5 mg 3 |
| month injection | month injection + Aricept ® | month injection + Aricept ® |
| | 5 mg Tab | 10 mg Tab |
| Lupron Depot ® 30 mg 4 | Lupron Depot ® 30 mg 4 | Lupron Depot ® 30 mg 4 |
| month injection | month injection + Aricept ® | month injection + Aricept ® |
| | 5 mg Tab | 10 mg Tab |
| Lupron Depot ® multidose | Lupron Depot ® multidose | Lupron Depot ® multidose |
| vials with 2.8 ml of 5 mg/ml | vials with 2.8 ml of 5 mg/ml | vials with 2.8 ml of 5 mg/ml |
| daily injections | daily injections + Aricept ® | daily injections + Aricept ® |
| | 5 mg Tab | 10 mg Tab |
| Viadur ™ 72 mg 12 month | Viadur ™ 72 mg 12 month | Viadur ™ 72 mg 12 month |
| implantation | implantation + Aricept ® | implantation + Aricept ® |
| | 5 mg Tab | 10 mg Tab |
| ELIGARD ® 7.5 mg 1 month | ELIGARD ® 7.5 mg 1 | ELIGARD ® 7.5 mg 1 |
| injection | month injection + Aricept ® | month injection + Aricept ® |
| | 5 mg Tab | 10 mg Tab |
| ELIGARD ® 22.5 mg 3 month | ELIGARD ® 22.5 mg 3 | ELIGARD ® 22.5 mg 3 |
| injection | month injection + Aricept ® | month injection + Aricept ® |
| | 5 mg Tab | 10 mg Tab |
| ELIGARD ® 30 mg 4 month | ELIGARD ® 30 mg 4 | ELIGARD ® 30 mg 4 |
| injection | month injection + Aricept ® | month injection + Aricept ® |
| | 5 mg Tab | 10 mg Tab |
| ELIGARD ® 45 mg 3 month | ELIGARD ® 45 mg 3 | ELIGARD ® 45 mg 3 |
| injection | month injection + Aricept ® | month injection + Aricept ® |
| | 5 mg Tab | 10 mg Tab |
| Zoladex ® 3.6 mg 1month | Zoladex ® 3.6 mg 1month + Aricept ® | Zoladex ® 3.6 mg 1month + Aricept ® |
| | 5 mg Tab | 10 mg Tab |
| Synarel ® 200 micrograms | Synarel ® 200 micrograms | Synarel ® 200 micrograms |
| twice daily | twice daily + Aricept ® | twice daily + Aricept ® |
| | 5 mg Tab | 10 mg Tab |
| Synarel ® daily intranasal | Synarel ® daily intranasal | Synarel ® daily intranasal |
| dosings for children and | dosings for children and | dosings for children and |
| adults range from 200 ug to | adults range from 200 ug to | adults range from 200 ug to |
| 1800 ug | 1800 ug + Aricept ® 5 mg | 1800 ug + Aricept ® 10 mg |
| | Tab | Tab |
| TRELSTAR ® DEPOT | TRELSTAR ® DEPOT | TRELSTAR ® DEPOT |
| 3.75 mg 1 month | 3.75 mg 1 month + Aricept ® | 3.75 mg 1 month + Aricept ® |
| | 5 mg Tab | 10 mg Tab |
| TRELSTAR ® LA 11.25 mg | TRELSTAR ® LA 11.25 mg | TRELSTAR ® LA 11.25 mg |
| 12 week injection | 12 week injection + Aricept ® | 12 week injection + Aricept ® |
| | 5 mg Tab | 10 mg Tab |
| SUPPRELIN ® 200 ug/ml | SUPPRELIN ® 200 ug/ml | SUPPRELIN ® 200 ug/ml |
| daily injection | daily injection + Aricept ® | daily injection + Aricept ® |
| | 5 mg Tab | 10 mg Tab |
| SUPPRELIN ® 500 ug/ml | SUPPRELIN ® 500 ug/ml | SUPPRELIN ® 500 ug/ml |
| daily injection | daily injection + Aricept ® | daily injection + Aricept ® |
| | 5 mg Tab | 10 mg Tab |
| SUPPRELIN ® 1000 ug/ml | SUPPRELIN ® 1000 ug/ml | SUPPRELIN ® 1000 ug/ml |
| daily injection | daily injection + Aricept ® | daily injection + Aricept ® |
| | 5 mg Tab | 10 mg Tab |
| SUPPRELIN ® LA 50 mg 12 | SUPPRELIN ® LA 50 mg | SUPPRELIN ® LA 50 mg |
| month implant | 12 month implant + Aricept ® | 12 month implant + Aricept ® |
| | 5 mg Tab | 10 mg Tab |
| VANTAS ® 50 mg 12 month | VANTAS ® 50 mg 12 | VANTAS ® 50 mg 12 |
| implant | month implant + Aricept ® | month implant + Aricept ® |
| | 5 mg Tab | 10 mg Tab |
| SUPREFACT ® 6.3 mg 2 | SUPREFACT ® 6.3 mg 2 | SUPREFACT ® 6.3 mg 2 |
| month implant | month implant + Aricept ® | month implant + Aricept ® |
| | 5 mg Tab | 10 mg Tab |
| SUPREFACT ® 500 ug every | SUPREFACT ® 500 ug | SUPREFACT ® 500 ug |
| 8 hours for 7 days followed | every 8 hours for 7 days | every 8 hours for 7 days |
| by 200 ug per day | followed by 200 ug per day + Aricept ® | followed by 200 ug per day + Aricept ® |
| | 5 mg Tab | 10 mg Tab |
| SUPREFACT ® 9.5 mg 3 | SUPREFACT ® 9.5 mg 3 | SUPREFACT ® 9.5 mg 3 |
| month implant | month implant + Aricept ® | month implant + Aricept ® |
| | 5 mg Tab | 10 mg Tab |
| Cetrotide ® 0.25 mg daily | Cetrotide ® 0.25 mg daily + Aricept ® | Cetrotide ® 0.25 mg daily + Aricept ® |
| | 5 mg Tab | 10 mg Tab |
| Cetrotide ® 3.0 mg every 4 | Cetrotide ® 3.0 mg every 4 | Cetrotide ® 3.0 mg every 4 |
| days | days + Aricept ® 5 mg Tab | days + Aricept ® 10 mg Tab |
| PLENAXIS ® 100 mg given | PLENAXIS ® 100 mg | PLENAXIS ® 100 mg |
| on days 1, 15 and 28 and | given on days 1, 15 and 28 | given on days 1, 15 and 28 |
| every 4 weeks afterward | and every 4 weeks | and every 4 weeks |
| | afterward + Aricept ® 5 mg | afterward + Aricept ® 10 mg |
| | Tab | Tab |
| Antagon ™ 250 ug daily | Antagon ™ 250 ug daily + Aricept ® | Antagon ™ 250 ug daily + Aricept ® |
| | 5 mg Tab | 10 mg Tab |
| Decapeptyl ® SR 3 mg 1 | Decapeptyl ® SR 3 mg 1 | Decapeptyl ® SR 3 mg 1 |
| month subcutaenous and | month subcutaenous and | month subcutaenous and |
| intramuscular injections | intramuscular injections + Aricept ® | intramuscular injections + Aricept ® |
| | 5 mg Tab | 10 mg Tab |
| Decapeptyl ® SR 11.25 mg 3 | Decapeptyl ® SR 11.25 mg | Decapeptyl ® SR 11.25 mg |
| month injection | 3 month injection + Aricept ® | 3 month injection + Aricept ® |
| | 5 mg Tab | 10 mg Tab |

TABLE B
Combination of Rivastigmine with selected GnRH-I analog products for use in the method of the invention.

| | Dosage Regimen | | | | |
|---|---|---|---|---|---|
| | Dosage Regimen | | | | |
| | AChE inhibitor Rivastigmine | | | | |
| | Rivastigmine | Rivastigmine | Rivastigmine | Rivastigmine | Rivastigmine |
| | (Exelon ® 1.5 mg | (Exelon ® 3 mg | (Exelon ® 4.5 mg | (Exelon ® 6 mg | (Exelon ® oral |
| GnRH-I | capsules twice | capsules twice | capsules twice | capsules twice | solution 1.5 mg |
| Analogs | daily) | daily) | daily) | daily) | twice daily) |
| Lupron Depot ® | Lupron Depot ® | Lupron Depot ® | Lupron Depot ® | Lupron Depot ® | Lupron Depot ® |
| 3.75 mg 1 month | 3.75 mg 1 month | 3.75 mg 1 month | 3.75 mg 1 month | 3.75 mg 1 month | 3.75 mg 1 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot ® | Lupron Depot ® | Lupron Depot ® | Lupron Depot ® | Lupron Depot ® | Lupron Depot ® |
| 7.5 mg 1 month | 7.5 mg 1 month | 7.5 mg 1 month | 7.5 mg 1 month | 7.5 mg 1 month | 7.5 mg 1 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot- | Lupron Depot- | Lupron Depot- | Lupron Depot- | Lupron Depot- | Lupron Depot- |
| PED ® 11.25 mg | PED ® 11.25 mg | PED ® 11.25 mg | PED ® 11.25 mg | PED ® 11.25 mg | PED ® 11.25 mg |
| 1 month | 1 month | 1 month | 1 month | 1 month | 1 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot- | Lupron Depot- | Lupron Depot- | Lupron Depot- | Lupron Depot- | Lupron Depot- |
| PED ® 15 mg | PED ® 15 mg | PED ® 15 mg | PED ® 15 mg | PED ® 15 mg | PED ® 15 mg |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot ® | Lupron Depot ® | Lupron Depot ® | Lupron Depot ® | Lupron Depot ® | Lupron Depot ® |
| 22.5 mg 3 month | 22.5 mg 3 month | 22.5 mg 3 month | 22.5 mg 3 month | 22.5 mg 3 month | 22.5 mg 3 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot ® | Lupron Depot ® | Lupron Depot ® | Lupron Depot ® | Lupron Depot ® | Lupron Depot ® |
| 30 mg 4 month | 30 mg 4 month | 30 mg 4 month | 30 mg 4 month | 30 mg 4 month | 30 mg 4 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Lupron Depot ® | Lupron Depot ® | Lupron Depot ® | Lupron Depot ® | Lupron Depot ® | Lupron Depot ® |
| multidoses vials | multidose vials | multidose vials | multidose vials | multidose vials | multidose vials |
| with 2.8 ml of | with 2.8 ml of | with 2.8 ml of | with 2.8 ml of | with 2.8 ml of | with 2.8 ml of |
| 5 mg/ml daily | 5 mg/ml daily | 5 mg/ml daily | 5 mg/ml daily | 5 mg/ml daily | 5 mg/ml daily |
| injections | injections + | injections + | injections + | injections + | injections + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Viadur ™ 72 mg | Viadur ™ 72 mg | Viadur ™ 72 mg | Viadur ™ 72 mg | Viadur ™ 72 mg | Viadur ™ 72 mg |
| 12 month | 12 month | 12 month | 12 month | 12 month | 12 month |
| implantation | implantation + | implantation + | implantation + | implantation + | implantation + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| ELIGARD ® | ELIGARD ® | ELIGARD ® | ELIGARD ® | ELIGARD ® | ELIGARD ® |
| 7.5 mg 1 month | 7.5 mg 1 month | 7.5 mg 1 month | 7.5 mg 1 month | 7.5 mg 1 month | 7.5 mg 1 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| ELIGARD ® | ELIGARD ® | ELIGARD ® | ELIGARD ® | ELIGARD ® | ELIGARD ® |
| 22.5 mg 3 month | 22.5 mg 3 month | 22.5 mg 3 month | 22.5 mg 3 month | 22.5 mg 3 month | 22.5 mg 3 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| ELIGARD ® | ELIGARD ® | ELIGARD ® | ELIGARD ® | ELIGARD ® | ELIGARD ® |
| 30 mg 4 month | 30 mg 4 month | 30 mg 4 month | 30 mg 4 month | 30 mg 4 month | 30 mg 4 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| ELIGARD ® | ELIGARD ® | ELIGARD ® | ELIGARD ® | ELIGARD ® | ELIGARD ® |
| 45 mg 3 month | 45 mg 3 month | 45 mg 3 month | 45 mg 3 month | 45 mg 3 month | 45 mg 3 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Zoladex ® | Zoladex ® | Zoladex ® | Zoladex ® | Zoladex ® | Zoladex ® |
| 3.6 mg 1 month | 3.6 mg 1 month + | 3.6 mg 1 month + | 3.6 mg 1 month + | 3.6 mg 1 month + | 3.6 mg 1 month + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Synarel ® 200 | Synarel ® 200 | Synarel ® 200 | Synarel ® 200 | Synarel ® 200 | Synarel ® 200 |
| micrograms | micrograms | micrograms | micrograms | micrograms | micrograms |
| twice daily | twice daily + | twice daily + | twice daily + | twice daily + | twice daily + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Synarel ® daily | Synarel ® daily | Synarel ® daily | Synarel ® daily | Synarel ® daily | Synarel ® daily |
| intranasal dosings | intranasal dosings | intranasal dosings | intranasal dosings | intranasal dosings | intranasal dosings |
| for children and | for children and | for children and | for children and | for children and | for children and |
| adults range | adults range | adults range | adults range | adults range | adults range |
| from 200 ug to | from 200 ug to | from 200 ug to | from 200 ug to | from 200 ug to | from 200 ug to |
| 1800 ug | 1800 ug + | 1800 ug + | 1800 ug + | 1800 ug + | 1800 ug + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| TRELSTAR ® | TRELSTAR ® | TRELSTAR ® | TRELSTAR ® | TRELSTAR ® | TRELSTAR ® |
| DEPOT | DEPOT | DEPOT | DEPOT | DEPOT | DEPOT |
| 3.75 mg 1 month | 3.75 mg 1 month + | 3.75 mg 1 month + | 3.75 mg 1 month + | 3.75 mg 1 month + | 3.75 mg 1 month + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| TRELSTAR ® | TRELSTAR ® | TRELSTAR ® | TRELSTAR ® | TRELSTAR ® | TRELSTAR ® |
| LA 11.25 mg | LA 11.25 mg | LA 11.25 mg | LA 11.25 mg | LA 11.25 mg | LA 11.25 mg |
| 12 week | 12 week | 12 week | 12 week | 12 week | 12 week |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPPRELIN ® | SUPPRELIN ® | SUPPRELIN ® | SUPPRELIN ® | SUPPRELIN ® | SUPPRELIN ® |
| 200 ug/ml daily | 200 ug/ml daily | 200 ug/ml daily | 200 ug/ml daily | 200 ug/ml daily | 200 ug/ml daily |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPPRELIN ® | SUPPRELIN ® | SUPPRELIN ® | SUPPRELIN ® | SUPPRELIN ® | SUPPRELIN ® |
| 500 ug/ml daily | 500 ug/ml daily | 500 ug/ml daily | 500 ug/ml daily | 500 ug/ml daily | 500 ug/ml daily |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPPRELIN ® | SUPPRELIN ® | SUPPRELIN ® | SUPPRELIN ® | SUPPRELIN ® | SUPPRELIN ® |
| 1000 ug/ml daily | 1000 ug/ml daily | 1000 ug/ml daily | 1000 ug/ml daily | 1000 ug/ml daily | 1000 ug/ml daily |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPPRELIN ® | SUPPRELIN ® | SUPPRELIN ® | SUPPRELIN ® | SUPPRELIN ® | SUPPRELIN ® |
| LA 50 mg 12 month | LA 50 mg 12 month | LA 50 mg 12 month | LA 50 mg 12 month | LA 50 mg 12 month | LA 50 mg 12 month |
| implant | implant + | implant + | implant + | implant + | implant + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| VANTAS ® | VANTAS ® | VANTAS ® | VANTAS ® | VANTAS ® | VANTAS ® |
| 50 mg 12 month | 50 mg 12 month | 50 mg 12 month | 50 mg 12 month | 50 mg 12 month | 50 mg 12 month |
| implant | implant + | implant + | implant + | implant + | implant + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPREFACT ® | SUPREFACT ® | SUPREFACT ® | SUPREFACT ® | SUPREFACT ® | SUPREFACT ® |
| 6.3 mg 2 month | 6.3 mg 2 month | 6.3 mg 2 month | 6.3 mg 2 month | 6.3 mg 2 month | 6.3 mg 2 month |
| implant | implant + | implant + | implant + | implant + | implant + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPREFACT ® | SUPREFACT ® | SUPREFACT ® | SUPREFACT ® | SUPREFACT ® | SUPREFACT ® |
| 500 ug every 8 | 500 ug every 8 | 500 ug every 8 | 500 ug every 8 | 500 ug every 8 | 500 ug every 8 |
| hours for 7 days | hours for 7 days | hours for 7 days | hours for 7 days | hours for 7 days | hours for 7 days |
| followed by | followed by | followed by | followed by | followed by | followed by |
| 200 ug per day | 200 ug per day + | 200 ug per day + | 200 ug per day + | 200 ug per day + | 200 ug per day + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| SUPREFACT ® | SUPREFACT ® | SUPREFACT ® | SUPREFACT ® | SUPREFACT ® | SUPREFACT ® |
| 9.5 mg 3 month | 9.5 mg 3 month | 9.5 mg 3 month | 9.5 mg 3 month | 9.5 mg 3 month | 9.5 mg 3 month |
| implant | implant + | implant + | implant + | implant + | implant + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Cetrotide ® | Cetrotide ® | Cetrotide ® | Cetrotide ® | Cetrotide ® | Cetrotide ® |
| 0.25 mg daily | 0.25 mg daily + | 0.25 mg daily + | 0.25 mg daily + | 0.25 mg daily + | 0.25 mg daily + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Cetrotide ® | Cetrotide ® | Cetrotide ® | Cetrotide ® | Cetrotide ® | Cetrotide ® |
| 3.0 mg every 4 | 3.0 mg every 4 | 3.0 mg every 4 | 3.0 mg every 4 | 3.0 mg every 4 | 3.0 mg every 4 |
| days | days + | days + | days + | days + | days + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| PLENAXIS ® | PLENAXIS ® | PLENAXIS ® | PLENAXIS ® | PLENAXIS ® | PLENAXIS ® |
| 100 mg given on | 100 mg given on | 100 mg given on | 100 mg given on | 100 mg given on | 100 mg given on |
| days 1, 15 and 28 | days 1, 15 and 28 | days 1, 15 and 28 | days 1, 15 and 28 | days 1, 15 and 28 | days 1, 15 and 28 |
| and every 4 weeks | and every 4 weeks | and every 4 weeks | and every 4 weeks | and every 4 weeks | and every 4 weeks |
| afterward | afterward + | afterward + | afterward + | afterward + | afterward + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Antagon ™ | Antagon ™ | Antagon ™ | Antagon ™ | Antagon ™ | Antagon ™ |
| 250 ug daily | 250 ug daily + | 250 ug daily + | 250 ug daily + | 250 ug daily + | 250 ug daily + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Decapeptyl ® | Decapeptyl ® | Decapeptyl ® | Decapeptyl ® | Decapeptyl ® | Decapeptyl ® |
| SR 3 mg 1 month | SR 3 mg 1 month | SR 3 mg 1 month | SR 3 mg 1 month | SR 3 mg 1 month | SR 3 mg 1 month |
| subcutaneous | subcutaenous | subcutaenous | subcutaenous | subcutaenous | subcutaenous |
| and intramuscular | and intramuscular | and intramuscular | and intramuscular | and intramuscular | and intramuscular |
| injections | injections + | injections + | injections + | injections + | injections + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |
| Decapeptyl ® | Decapeptyl ® | Decapeptyl ® | Decapeptyl ® | Decapeptyl ® | Decapeptyl ® |
| SR 11.25 mg 3 month | SR 11.25 mg 3 month | SR 11.25 mg 3 month | SR 11.25 mg 3 month | SR 11.25 mg 3 month | SR 11.25 mg 3 month |
| injection | injection + | injection + | injection + | injection + | injection + |
| | Exelon ® 1.5 mg | Exelon ® 3 mg | Exelon ® 4.5 mg | Exelon ® 6 mg | Exelon ® 1.5 mg |
| | Cap | Cap | Cap | Cap | oral solution |

TABLE C
Combination of Galantamine with selected GnRH-I analog products for use in
the method of the invention.

| | Dosage Regimen | | |
|---|---|---|---|
| Dosage | AChE inhibitor Galantamine | | |
| Regimen | Galantamine | Galantamine | Galantamine |
| GnRH-I | (Reminyl ® 8 mg | (Reminyl ® 16 mg | (Reminyl ® 24 mg tablets |
| Analogs | tablets twice daily) | tablets twice daily) | twice daily) |
| Lupron Depot ® | Lupron Depot ® | Lupron Depot ® | Lupron Depot ® 3.75 mg 1 |
| 3.75 mg 1 | 3.75 mg 1 month | 3.75 mg 1 month | month injection + Reminyl ® |
| month injection | injection + Reminyl ® | injection + Reminyl ® | 24 mg tablets |
| | 8 mg tablets | 16 mg tablets | |
| Lupron Depot ® | Lupron Depot ® | Lupron Depot ® | Lupron Depot ® 7.5 mg 1 |
| 7.5 mg 1 month | 7.5 mg 1 month | 7.5 mg 1 month | month injection + Reminyl ® |
| injection | injection + Reminyl ® | injection + Reminyl ® | 24 mg tablets |
| | 8 mg tablets | 16 mg tablets | |
| Lupron Depot- | Lupron Depot- | Lupron Depot- | Lupron Depot-PED ® |
| PED ® 11.25 mg | PED ® 11.25 mg 1 | PED ® 11.25 mg 1 | 11.25 mg 1 month injection + Reminyl ® |
| 1 month | month injection + Reminyl ® | month injection + Reminyl ® | 24 mg tablets |
| injection | 8 mg | 16 mg | |
| | tablets | tablets | |
| Lupron Depot- | Lupron Depot-PED ® | Lupron Depot-PED ® | Lupron Depot-PED ® 15 mg |
| PED ® 15 mg | 15 mg injection + Reminyl ® | 15 mg injection + Reminyl ® | injection + Reminyl ® 24 mg |
| injection | 8 mg | 16 mg | tablets |
| | tablets | tablets | |
| Lupron Depot ® | Lupron Depot ® | Lupron Depot ® | Lupron Depot ® 22.5 mg 3 |
| 22.5 mg 3 | 22.5 mg 3 month | 22.5 mg 3 month | month injection + Reminyl ® |
| month injection | injection + Reminyl ® | injection + Reminyl ® | 24 mg tablets |
| | 8 mg tablets | 16 mg tablets | |
| Lupron Depot ® | Lupron Depot ® | Lupron Depot ® | Lupron Depot ® 30 mg 4 |
| 30 mg 4 month | 30 mg 4 month | 30 mg 4 month | month injection + Reminyl ® |
| injection | injection + Reminyl ® | injection + Reminyl ® | 24 mg tablets |
| | 8 mg tablets | 16 mg tablets | |
| Lupron Depot ® | Lupron Depot ® | Lupron Depot ® | Lupron Depot ® multidose |
| multidose vials | multidose vials with | multidose vials with | vials with 2.8 ml of 5 mg/ml |
| with 2.8 ml of | 2.8 ml of 5 mg/ml | 2.8 ml of 5 mg/ml | daily injections + Reminyl ® |
| 5 mg/ml daily | daily injections + Reminyl ® | daily injections + Reminyl ® | 24 mg tablets |
| injections | 8 mg | 16 mg | |
| | tablets | tablets | |
| Viadur ™ 72 mg | Viadur ™ 72 mg 12 | Viadur ™ 72 mg 12 | Viadur ™ 72 mg 12 month |
| 12 month | month implantation + Reminyl ® | month implantation + Reminyl ® | implantation + Reminyl ® |
| implantation | 8 mg | 16 mg | 24 mg tablets |
| | tablets | tablets | |
| ELIGARD ® | ELIGARD ® 7.5 mg 1 | ELIGARD ® 7.5 mg 1 | ELIGARD ® 7.5 mg 1 month |
| 7.5 mg 1 month | month injection + Reminyl ® | month injection + Reminyl ® | injection + Reminyl ® 24 mg |
| injection | 8 mg | 16 mg | tablets |
| | tablets | tablets | |
| ELIGARD ® | ELIGARD ® 22.5 mg | ELIGARD ® 22.5 mg | ELIGARD ® 22.5 mg 3 month |
| 22.5 mg 3 | 3 month injection + Reminyl ® | 3 month injection + Reminyl ® | injection + Reminyl ® 24 mg |
| month injection | 8 mg | 16 mg | tablets |
| | tablets | tablets | |
| ELIGARD ® | ELIGARD ® 30 mg 4 | ELIGARD ® 30 mg 4 | ELIGARD ® 30 mg 4 month |
| 30 mg 4 month | month injection + Reminyl ® | month injection + Reminyl ® | injection + Reminyl ® 24 mg |
| injection | 8 mg | 16 mg | tablets |
| | tablets | tablets | |
| ELIGARD ® | ELIGARD ® 45 mg 3 | ELIGARD ® 45 mg 3 | ELIGARD ® 45 mg 3 month |
| 45 mg 3 month | month injection + Reminyl ® | month injection + Reminyl ® | injection + Reminyl ® 24 mg |
| injection | 8 mg | 16 mg | tablets |
| | tablets | tablets | |
| Zoladex ® | Zoladex ® 3.6 mg | Zoladex ® 3.6 mg | Zoladex ® 3.6 mg 1month + Reminyl ® |
| 3.6 mg 1month | 1month + Reminyl ® | 1month + Reminyl ® | 24 mg tablets |
| | 8 mg tablets | 16 mg tablets | |
| Synarel ® 200 | Synarel ® 200 | Synarel ® 200 | Synarel ® 200 micrograms |
| micrograms | micrograms twice | micrograms twice | twice daily + Reminyl ® 24 mg |
| twice daily | daily + Reminyl ® | daily + Reminyl ® | tablets |
| | 8 mg tablets | 16 mg tablets | |
| Synarel ® daily | Synarel ® daily | Synarel ® daily | Synarel ® daily intranasal |
| intranasal | intranasal dosings for | intranasal dosings for | dosings for children and |
| dosings for | children and adults | children and adults | adults range from 200 ug to |
| children and | range from 200 ug to | range from 200 ug to | 1800 ug + Reminyl ® 24 mg |
| adults range | 1800 ug + Reminyl ® | 1800 ug + Reminyl ® | tablets |
| from 200 ug to | 8 mg tablets | 16 mg tablets | |
| 1800 ug | | | |
| TRELSTAR ® | TRELSTAR ® | TRELSTAR ® | TRELSTAR ® DEPOT |
| DEPOT | DEPOT 3.75 mg 1 | DEPOT 3.75 mg 1 | 3.75 mg 1 month + Reminyl ® |
| 3.75 mg 1 | month + Reminyl ® | month + Reminyl ® | 24 mg tablets |
| month | 8 mg tablets | 16 mg tablets | |
| TRELSTAR ® | TRELSTAR ® LA | TRELSTAR ® LA | TRELSTAR ® LA 11.25 mg |
| LA 11.25 mg | 11.25 mg 12 week | 11.25 mg 12 week | 12 week injection + Reminyl ® |
| 12 week | injection + Reminyl ® | injection + Reminyl ® | 24 mg tablets |
| injection | 8 mg tablets | 16 mg tablets | |
| SUPPRELIN ® | SUPPRELIN ® | SUPPRELIN ® | SUPPRELIN ® 200 ug/ml |
| 200 ug/ml daily | 200 ug/ml daily | 200 ug/ml daily | daily injection + Reminyl ® |
| injection | injection + Reminyl ® | injection + Reminyl ® | 24 mg tablets twice daily |
| | 8 mg tablets twice | 16 mg tablets twice | |
| | daily | daily | |
| SUPPRELIN ® | SUPPRELIN ® | SUPPRELIN ® | SUPPRELIN ® 500 ug/ml |
| 500 ug/ml daily | 500 ug/ml daily | 500 ug/ml daily | daily injection + Reminyl ® |
| injection | injection + Reminyl ® | injection + Reminyl ® | 24 mg tablets twice daily |
| | 8 mg tablets twice | 16 mg | |
| | daily | tablets twice daily | |
| SUPPRELIN ® | SUPPRELIN ® | SUPPRELIN ® | SUPPRELIN ® 1000 ug/ml |
| 1000 ug/ml | 1000 ug/ml daily | 1000 ug/ml daily | daily injection + Reminyl ® |
| daily injection | injection + Reminyl ® | injection + Reminyl ® | 24 mg tablets twice daily |
| | 8 mg tablets twice | 16 mg tablets twice | |
| | daily | daily | |
| SUPPRELIN ® | SUPPRELIN ® LA | SUPPRELIN ® LA | SUPPRELIN ® LA 50 mg 12 |
| LA 50 mg 12 | 50 mg 12 month | 50 mg 12 month | month implant + Reminyl ® |
| month implant | implant + Reminyl ® | implant + Reminyl ® | 24 mg tablets twice daily |
| | 8 mg tablets twice | 16 mg tablets twice | |
| | daily | daily | |
| VANTAS ® | VANTAS ® 50 mg 12 | VANTAS ® 50 mg 12 | VANTAS ® 50 mg 12 month |
| 50 mg 12 month | month implant + Reminyl ® | month implant + Reminyl ® | implant + Reminyl ® 24 mg |
| implant | 8 mg | 16 mg | tablets twice daily |
| | tablets twice daily | tablets twice daily | |
| SUPREFACT ® | SUPREFACT ® | SUPREFACT ® | SUPREFACT ® 6.3 mg 2 |
| 6.3 mg 2 month | 6.3 mg 2 month | 6.3 mg 2 month | month implant + Reminyl ® |
| implant | implant + Reminyl ® | implant + Reminyl ® | 24 mg tablets twice daily |
| | 8 mg tablets twice | 16 mg tablets twice | |
| | daily | daily | |
| SUPREFACT ® | SUPREFACT ® | SUPREFACT ® | SUPREFACT ® 500 ug every |
| 500 ug every 8 | 500 ug every 8 hours | 500 ug every 8 hours | 8 hours for 7 days followed |
| hours for 7 | for 7 days followed | for 7 days followed | by 200 ug per day + Reminyl ® |
| days followed | by 200 ug per day + Reminyl ® | by 200 ug per day + Reminyl ® | 24 mg tablets twice |
| by 200 ug per | 8 mg | 16 mg | daily |
| day | tablets twice daily | tablets twice daily | |
| SUPREFACT ® | SUPREFACT ® | SUPREFACT ® | SUPREFACT ® 9.5 mg 3 |
| 9.5 mg 3 month | 9.5 mg 3 month | 9.5 mg 3 month | month implant + Reminyl ® |
| implant | implant + Reminyl ® | implant + Reminyl ® | 24 mg tablets twice daily |
| | 8 mg tablets twice | 16 mg tablets twice | |
| | daily | daily | |
| Cetrotide ® | Cetrotide ® 0.25 mg | Cetrotide ® 0.25 mg | Cetrotide ® 0.25 mg daily + Reminyl ® |
| 0.25 mg daily | daily + Reminyl ® | daily + Reminyl ® | 24 mg tablets twice |
| | 8 mg tablets twice | 16 mg tablets twice | daily |
| | daily | daily | |
| Cetrotide ® | Cetrotide ® 3.0 mg | Cetrotide ® 3.0 mg | Cetrotide ® 3.0 mg every 4 |
| 3.0 mg every 4 | every 4 days + Reminyl ® | every 4 days + Reminyl ® | days + Reminyl ® 24 mg |
| days | 8 mg | 16 mg | tablets twice daily |
| | tablets twice daily | tablets twice daily | |
| PLENAXIS ® | PLENAXIS ® 100 mg | PLENAXIS ® 100 mg | PLENAXIS ® 100 mg given |
| 100 mg given | given on days 1, 15 | given on days 1, 15 | on days 1, 15 and 28 and every |
| on days 1, 15 | and 28 and every 4 | and 28 and every 4 | 4 weeks afterward + Reminyl ® |
| and 28 and | weeks afterward + Reminyl ® | weeks afterward + Reminyl ® | 24 mg tablets twice |
| every 4 weeks | 8 mg | 16 mg | daily |
| afterward | tablets twice daily | tablets twice daily | |
| Antagon ™ | Antagon ™ 250 ug | Antagon ™ 250 ug | Antagon ™ 250 ug daily + Reminyl ® |
| 250 ug daily | daily + Reminyl ® | daily + Reminyl ® | 24 mg tablets twice |
| | 8 mg tablets twice | 16 mg tablets twice | daily |
| | daily | daily | |
| Decapeptyl ® | Decapeptyl ® SR | Decapeptyl ® SR 3 mg | Decapeptyl ® SR 3 mg 1 |
| SR 3 mg 1 | 3 mg 1 month | 1 month | month subcutaenous and |
| month | subcutaenous and | subcutaenous and | intramuscular injections + Reminyl ® |
| subcutaenous | intramuscular | intramuscular | 24 mg tablets twice |
| and | injections + Reminyl ® | injections + Reminyl ® | daily |
| intramuscular | 8 mg | 16 mg | |
| injections | tablets twice daily | tablets twice daily | |
| Decapeptyl ® | Decapeptyl ® SR | Decapeptyl ® SR | Decapeptyl ® SR 11.25 mg 3 |
| SR 11.25 mg 3 | 11.25 mg 3 month | 11.25 mg 3 month | month injection + Reminyl ® |
| month | injection + Reminyl ® | injection + Reminyl ® | 24 mg tablets twice daily |
| injection | 8 mg tablets twice | 16 mg tablets twice | |
| | daily | daily | |

TABLE D
Combination of Tacrine with selected GnRH-I analog products according to
various embodiments of the invention.

| Dosage | AChE inhibitor Cognex | | | |
|---|---|---|---|---|
| Regimen | Tetrahydroaminoacridine- | Tetrahydroaminoacridine- | Tetrahydroaminoacridine- | Tetrahydroaminoacridine- |
| GnRH-I | Tacrine (Cognex ® 10 mg | Tacrine (Cognex ® 20 mg | Tacrine (Cognex ® 30 mg | Tacrine (Cognex ® 40 mg |
| Analogs | QID) | QID) | QID) | QID) |
| Lupron Depot ® | Lupron Depot ® 3.75 mg 1 | Lupron Depot ® 3.75 mg 1 | Lupron Depot ® 3.75 mg 1 | Lupron Depot ® 3.75 mg 1 |
| 3.75 mg 1 | month injection + Cognex ® | month injection + Cognex ® | month injection + Cognex ® | month injection + Cognex ® |
| month injection | 10 mg | 20 mg | 30 mg | 40 mg |
| Lupron Depot ® | Lupron Depot ® 7.5 mg 1 | Lupron Depot ® 7.5 mg 1 | Lupron Depot ® 7.5 mg 1 | Lupron Depot ® 7.5 mg 1 |
| 7.5 mg 1 month | month injection + Cognex ® | month injection + Cognex ® | month injection + Cognex ® | month injection + Cognex ® |
| injection | 10 mg | 20 mg | 30 mg | 40 mg |
| Lupron Depot- | Lupron Depot-PED ® | Lupron Depot-PED ® | Lupron Depot-PED ® | Lupron Depot-PED ® |
| PED ® 11.25 mg | 11.25 mg 1 month | 11.25 mg 1 month | 11.25 mg 1 month | 11.25 mg 1 month |
| 1 month | injection + Cognex ® | injection + Cognex ® | injection + Cognex ® | injection + Cognex ® |
| injection | 10 mg | 20 mg | 30 mg | 40 mg |
| Lupron Depot- | Lupron Depot-PED ® | Lupron Depot-PED ® | Lupron Depot-PED ® | Lupron Depot-PED ® |
| PED ® 15 mg | 15 mg injection + Cognex ® | 15 mg injection + Cognex ® | 15 mg injection + Cognex ® | 15 mg injection + Cognex ® |
| injection | 10 mg | 20 mg | 30 mg | 40 mg |
| Lupron Depot ® | Lupron Depot ® 22.5 mg 3 | Lupron Depot ® 22.5 mg 3 | Lupron Depot ® 22.5 mg 3 | Lupron Depot ® 22.5 mg 3 |
| 22.5 mg 3 | month injection + Cognex ® | month injection + Cognex ® | month injection + Cognex ® | month injection + Cognex ® |
| month injection | 10 mg | 20 mg | 30 mg | 40 mg |
| Lupron Depot ® | Lupron Depot ® 30 mg 4 | Lupron Depot ® 30 mg 4 | Lupron Depot ® 30 mg 4 | Lupron Depot ® 30 mg 4 |
| 30 mg 4 month | month injection + Cognex ® | month injection + Cognex ® | month injection + Cognex ® | month injection + Cognex ® |
| injection | 10 mg | 20 mg | 30 mg | 40 mg |
| Lupron Depot ® | Lupron Depot ® | Lupron Depot ® | Lupron Depot ® | Lupron Depot ® |
| multidose vials | multidose vials with | multidose vials with | multidose vials with | multidose vials with |
| with 2.8 ml of | 2.8 ml of 5 mg/ml daily | 2.8 ml of 5 mg/ml daily | 2.8 ml of 5 mg/ml daily | 2.8 ml of 5 mg/ml daily |
| 5 mg/ml daily | injections + Cognex ® | injections + Cognex ® | injections + Cognex ® | injections + Cognex ® |
| injections | 10 mg | 20 mg | 30 mg | 40 mg |
| Viadur ™ 72 mg | Viadur ™ 72 mg 12 month | Viadur ™ 72 mg 12 month | Viadur ™ 72 mg 12 month | Viadur ™ 72 mg 12 month |
| 12 month | implantation + Cognex ® | implantation + Cognex ® | implantation + Cognex ® | implantation + Cognex ® |
| implantation | 10 mg | 20 mg | 30 mg | 40 mg |
| ELIGARD ® | ELIGARD ® 7.5 mg 1 | ELIGARD ® 7.5 mg 1 | ELIGARD ® 7.5 mg 1 | ELIGARD ® 7.5 mg 1 |
| 7.5 mg 1 month | month injection + Cognex ® | month injection + Cognex ® | month injection + Cognex ® | month injection + Cognex ® |
| injection | 10 mg | 20 mg | 30 mg | 40 mg |
| ELIGARD ® | ELIGARD ® 22.5 mg 3 | ELIGARD ® 22.5 mg 3 | ELIGARD ® 22.5 mg 3 | ELIGARD ® 22.5 mg 3 |
| 22.5 mg 3 | month injection + Cognex ® | month injection + Cognex ® | month injection + Cognex ® | month injection + Cognex ® |
| month injection | 10 mg | 20 mg | 30 mg | 40 mg |
| ELIGARD ® | ELIGARD ® 30 mg 4 | ELIGARD ® 30 mg 4 | ELIGARD ® 30 mg 4 | ELIGARD ® 30 mg 4 |
| 30 mg 4 month | month injection + Cognex ® | month injection + Cognex ® | month injection + Cognex ® | month injection + Cognex ® |
| injection | 10 mg | 20 mg | 30 mg | 40 mg |
| ELIGARD ® | ELIGARD ® 45 mg 3 | ELIGARD ® 45 mg 3 | ELIGARD ® 45 mg 3 | ELIGARD ® 45 mg 3 |
| 45 mg 3 month | month injection + Cognex ® | month injection + Cognex ® | month injection + Cognex ® | month injection + Cognex ® |
| injection | 10 mg | 20 mg | 30 mg | 40 mg |
| Zoladex ® | Zoladex ® 3.6 mg 1 month + | Zoladex ® 3.6 mg 1 month + | Zoladex ® 3.6 mg 1 month + | Zoladex ® 3.6 mg 1 month + |
| 3.6 mg 1 month | Cognex ® 10 mg | Cognex ® 20 mg | Cognex ® 30 mg | Cognex ® 40 mg |
| Synarel ® 200 | Synarel ® 200 | Synarel ® 200 | Synarel ® 200 | Synarel ® 200 |
| micrograms | micrograms twice daily + | micrograms twice daily + | micrograms twice daily + | micrograms twice daily + |
| twice daily | Cognex ® 10 mg | Cognex ® 20 mg | Cognex ® 30 mg | Cognex ® 40 mg |
| Synarel ® daily | Synarel ® daily intranasal | Synarel ® daily intranasal | Synarel ® daily intranasal | Synarel ® daily intranasal |
| intranasal | dosings for children and | dosings for children and | dosings for children and | dosings for children and |
| dosings for | adults range from 200 ug | adults range from 200 ug | adults range from 200 ug | adults range from 200 ug |
| children and | to 1800 ug + Cognex ® | to 1800 ug + Cognex ® | to 1800 ug + Cognex ® | to 1800 ug + Cognex ® |
| adults range | 10 mg | 20 mg | 30 mg | 40 mg |
| from 200 ug to | | | | |
| 1800 ug | | | | |
| TRELSTAR ® | TRELSTAR ® DEPOT | TRELSTAR ® DEPOT | TRELSTAR ® DEPOT | TRELSTAR ® DEPOT |
| DEPOT | 3.75 mg 1 month + Cognex ® | 3.75 mg 1 month + Cognex ® | 3.75 mg 1 month + Cognex ® | 3.75 mg 1 month + Cognex ® |
| 3.75 mg 1 | 10 mg | 20 mg | 30 mg | 40 mg |
| month | | | | |
| TRELSTAR ® | TRELSTAR ® LA 11.25 mg | TRELSTAR ® LA 11.25 mg | TRELSTAR ® LA 11.25 mg | TRELSTAR ® LA 11.25 mg |
| LA 11.25 mg | 12 week injection + | 12 week injection + | 12 week injection + | 12 week injection + |
| 12 week | Cognex ® 10 mg | Cognex ® 20 mg | Cognex ® 30 mg | Cognex ® 40 mg |
| injection | | | | |
| SUPPRELIN ® | SUPPRELIN ® 200 ug/ml | SUPPRELIN ® 200 ug/ml | SUPPRELIN ® 200 ug/ml | SUPPRELIN ® 200 ug/ml |
| 200 ug/ml daily | daily injection + Cognex ® | daily injection + Cognex ® | daily injection + Cognex ® | daily injection + Cognex ® |
| injection | 10 mg | 20 mg | 30 mg | 40 mg |
| SUPPRELIN ® | SUPPRELIN ® 500 ug/ml | SUPPRELIN ® 500 ug/ml | SUPPRELIN ® 500 ug/ml | SUPPRELIN ® 500 ug/ml |
| 500 ug/ml daily | daily injection + Cognex ® | daily injection + Cognex ® | daily injection + Cognex ® | daily injection + Cognex ® |
| injection | 10 mg | 20 mg | 30 mg | 40 mg |
| SUPPRELIN ® | SUPPRELIN ® | SUPPRELIN ® | SUPPRELIN ® | SUPPRELIN ® |
| 1000 ug/ml | 1000 ug/ml daily injection + | 1000 ug/ml daily injection + | 1000 ug/ml daily injection + | 1000 ug/ml daily injection + |
| daily injection | Cognex ® 10 mg | Cognex ® 20 mg | Cognex ® 30 mg | Cognex ® 40 mg |
| SUPPRELIN ® | SUPPRELIN ® LA 50 mg | SUPPRELIN ® LA 50 mg | SUPPRELIN ® LA 50 mg | SUPPRELIN ® LA 50 mg |
| LA 50 mg 12 | 12 month implant + | 12 month implant + Cognex ® | 12 month implant + Cognex ® | 12 month implant + Cognex ® |
| month implant | Cognex ® 10 mg | 20 mg | 30 mg | 40 mg |
| VANTAS ® | VANTAS ® 50 mg 12 | VANTAS ® 50 mg 12 | VANTAS ® 50 mg 12 | VANTAS ® 50 mg 12 |
| 50 mg 12 month | month implant + Cognex ® | month implant + Cognex ® | month implant + Cognex ® | month implant + Cognex ® |
| implant | 10 mg | 20 mg | 30 mg | 40 mg |
| SUPREFACT ® | SUPREFACT ® 6.3 mg 2 | SUPREFACT ® 6.3 mg 2 | SUPREFACT ® 6.3 mg 2 | SUPREFACT ® 6.3 mg 2 |
| 6.3 mg 2 month | month implant + Cognex ® | month implant + Cognex ® | month implant + Cognex ® | month implant + Cognex ® |
| implant | 10 mg | 20 mg | 30 mg | 40 mg |
| SUPREFACT ® | SUPREFACT ® 500 ug | SUPREFACT ® 500 ug | SUPREFACT ® 500 ug | SUPREFACT ® 500 ug |
| 500 ug every 8 | every 8 hours for 7 days | every 8 hours for 7 days | every 8 hours for 7 days | every 8 hours for 7 days |
| hours for 7 | followed by 200 ug per | followed by 200 ug per | followed by 200 ug per | followed by 200 ug per |
| days followed | day + Cognex ® 10 mg | day + Cognex ® 20 mg | day + Cognex ® 30 mg | day + Cognex ® 40 mg |
| by 200 ug per | | | | |
| day | | | | |
| SUPREFACT ® | SUPREFACT ® 9.5 mg 3 | SUPREFACT ® 9.5 mg 3 | SUPREFACT ® 9.5 mg 3 | SUPREFACT ® 9.5 mg 3 |
| 9.5 mg 3 month | month implant + Cognex ® | month implant + Cognex ® | month implant + Cognex ® | month implant + Cognex ® |
| implant | 10 mg | 20 mg | 30 mg | 40 mg |
| Cetrotide ® | Cetrotide ® 0.25 mg daily + | Cetrotide ® 0.25 mg daily + | Cetrotide ® 0.25 mg daily + | Cetrotide ® 0.25 mg daily + |
| 0.25 mg daily | Cognex ® 10 mg | Cognex ® 20 mg | Cognex ® 30 mg | Cognex ® 40 mg |
| Cetrotide ® | Cetrotide ® 3.0 mg every 4 | Cetrotide ® 3.0 mg every 4 | Cetrotide ® 3.0 mg every 4 | Cetrotide ® 3.0 mg every 4 |
| 3.0 mg every 4 | days + Cognex ® 10 mg | days + Cognex ® 20 mg | days + Cognex ® 30 mg | days + Cognex ® 40 mg |
| days | | | | |
| PLENAXIS ® | PLENAXIS ® 100 mg | PLENAXIS ® 100 mg | PLENAXIS ® 100 mg | PLENAXIS ® 100 mg |
| 100 mg given | given on days 1, 15 and 28 | given on days 1, 15 and 28 | given on days 1, 15 and 28 | given on days 1, 15 and 28 |
| on days 1, 15 | and every 4 weeks | and every 4 weeks | and every 4 weeks | and every 4 weeks |
| and 28 and | afterward + Cognex ® | afterward + Cognex ® | afterward + Cognex ® | afterward + Cognex ® |
| every 4 weeks | 10 mg | 20 mg | 30 mg | 40 mg |
| afterward | | | | |
| Antagon ™ | Antagon ™ 250 ug daily + | Antagon ™ 250 ug daily + | Antagon ™ 250 ug daily + | Antagon ™ 250 ug daily + |
| 250 ug daily | Cognex ® 10 mg | Cognex ® 20 mg | Cognex ® 30 mg | Cognex ® 40 mg |
| Decapeptyl ® | Decapeptyl ® SR 3 mg 1 | Decapeptyl ® SR 3 mg 1 | Decapeptyl ® SR 3 mg 1 | Decapeptyl ® SR 3 mg 1 |
| SR 3 mg 1 | month subcutaenous and | month subcutaenous and | month subcutaenous and | month subcutaenous and |
| month | intramuscular injections + | intramuscular injections + | intramuscular injections + | intramuscular injections + |
| subcutaenous | Cognex ® 10 mg | Cognex ® 20 mg | Cognex ® 30 mg | Cognex ® 40 mg |
| and | | | | |
| intramuscular | | | | |
| injections | | | | |
| Decapeptyl ® | Decapeptyl ® SR | Decapeptyl ® SR | Decapeptyl ® SR | Decapeptyl ® SR |
| SR 11.25 mg 3 | 11.25 mg 3 month | 11.25 mg 3 month | 11.25 mg 3 month | 11.25 mg 3 month |
| month | injection + Cognex ® | injection + Cognex ® | injection + Cognex ® | injection + Cognex ® |
| injection | 10 mg | 20 mg | 30 mg | 40 mg |

As further described in Table E below, combination therapies according to the present invention may also include the administration of one or more N-methyl-D-aspartate (NMDA) antagonists. For example, combined use of one or more GnRH-I analogs such as leuprolide, goserelin, triptorelin, nafarelin, histrelin, buserelin, cetrorelix, abarelix, or ganirelix, comprising the free-base or acetate, alkyl carboxylate, benzoate, aryl carboxylate, pamoate, hydrochloride, hydrobromide, sulfate, oxalate, mesylate, or other salt forms including polymer-bound-anion salt forms with the use of one or more N-methyl-D-aspartate (NMDA) antagonist such as NAMENDA® or Ebixa® comprising the free-base or acetate, alkyl carboxylate, benzoate, aryl carboxylate, pamoate, hydrochloride, hydrobromide, sulfate, oxalate, mesylate, or other salt forms including polymer-bound-anion salt forms has the net effect of reducing the number of neurons that die in AD brains.

TABLE E
Combination of Memantine (Ebixa ®) with selected GnRH-I analog products
for use in methods according to embodiments of the invention.

| | Dosing Regimen |
|---|---|
| | Memantine (Ebixa ®) |
| Dosing Regimen | Memantine (Ebixa ® 10 mg |
| GnRH-I Analogs | tablet twice daily) |
| Lupron Depot ® 3.75 mg 1 month injection | Lupron Depot ® 3.75 mg 1 month injection + Memantine |
| | (Ebixa ® 10 mg) |
| Lupron Depot ® 7.5 mg 1 month injection | Lupron Depot ® 7.5 mg 1 month injection + Memantine |
| | (Ebixa ® 10 mg) |
| Lupron Depot-PED ® 11.25 mg 1 month | Lupron Depot-PED ® 11.25 mg 1 month |
| injection | injection + Memantine(Ebixa ® 10 mg) |
| Lupron Depot-PED ® 15 mg injection | Lupron Depot-PED ® 15 mg injection + Memantine |
| | (Ebixa ® 10 mg) |
| Lupron Depot ® 22.5 mg 3 month injection | Lupron Depot ® 22.5 mg 3 month injection + Memantine |
| | (Ebixa ® 10 mg) |
| Lupron Depot ® 30 mg 4 month injection | Lupron Depot ® 30 mg 4 month injection + Memantine |
| | (Ebixa ® 10 mg) |
| Lupron Depot ® multidose vials with 2.8 ml | Lupron Depot ® multidose vials with 2.8 ml |
| of 5 g/ml daily injections | of 5 g/ml daily injections + Memantine |
| | (Ebixa ® 10 mg) |
| Viadur ™ 72 mg 12 month implantation | Viadur ™ 72 mg 12 month implantation + Memantine |
| | Memantine(Ebixa ® 10 mg) |
| ELIGARD ® 7.5 mg 1 month injection | ELIGARD ® 7.5 mg 1 month injection + Memantine |
| | (Ebixa ® 10 mg) |
| ELIGARD ® 22.5 mg 3 month injection | ELIGARD ® 22.5 mg 3 month injection + Memantine |
| | (Ebixa ® 10 mg) |
| ELIGARD ® 30 mg 4 month injection | ELIGARD ® 30 mg 4 month injection + Memantine |
| | (Ebixa ® 10 mg) |
| ELIGARD ® 45 mg 3 month injection | ELIGARD ® 45 mg 3 month injection + Memantine |
| | (Ebixa ® 10 mg) |
| Zoladex ® 3.6 mg 1 month | Zoladex ® 3.6 mg 1month + Doepezil + Memantine |
| | (Ebixa ® 10 mg) |
| Synarel ® 200 micrograms twice daily | Synarel ® 200 micrograms twice daily + Memantine |
| | (Ebixa ® 10 mg) |
| Synarel ® daily intranasal dosings for | Synarel ® daily intranasal dosings for |
| children and adults range from 200 ug to | children and adults range from 200 ug to |
| 1800 ug | 1800 ug + Memantine(Ebixa ® 10 mg) |
| TRELSTAR ® DEPOT 3.75 mg 1 month | TRELSTAR ® DEPOT 3.75 mg 1 month + Memantine |
| | (Ebixa ® 10 mg) |
| TRELSTAR ® LA 11.25 mg 12 week | TRELSTAR ® LA 11.25 mg 12 week |
| injection | injection + Memantine(Ebixa ® 10 mg) |
| GnRH-I Analogs | Memantine (Ebixa ® 10 mg twice daily) |
| SUPPRELIN ® 200 ug/ml daily injection | SUPPRELIN ® 200 ug/ml daily injection + Memantine |
| | (Ebixa ® 10 mg) |
| SUPPRELIN ® 500 ug/ml daily injection | SUPPRELIN ® 500 ug/ml daily injection + Memantine |
| | (Ebixa ® 10 mg) |
| SUPPRELIN ® 1000 ug/ml daily injection | SUPPRELIN ® 1000 ug/ml daily injection + Memantine |
| | (Ebixa ® 10 mg) |
| SUPPRELIN ® LA 50 mg 12 month | SUPPRELIN ® LA 50 mg 12 month |
| implant | implant + Memantine(Ebixa ® 10 mg) |
| VANTAS ® 50 mg 12 month implant | VANTAS ® 50 mg 12 month implant + Memantine |
| | (Ebixa ® 10 mg) |
| SUPREFACT ® 6.3 mg 2 month implant | SUPREFACT ® 6.3 mg 2 month implant + Memantine |
| | (Ebixa ® 10 mg) |
| SUPREFACT ® 500 ug every 8 hours for 7 | SUPREFACT ® 500 ug every 8 hours for 7 |
| days followed by 200 ug per day | days followed by 200 ug per day + Memantine |
| | (Ebixa ® 10 mg) |
| SUPREFACT ® 9.5 mg 3 month implant | SUPREFACT ® 9.5 mg 3 month implant + Memantine |
| | (Ebixa ® 10 mg) |
| Cetrotide ® 0..25 mg daily | Cetrotide ® 0..25 mg daily + Memantine |
| | (Ebixa ® 10 mg) |
| Cetrotide ® 3.0 mg every 4 days | Cetrotide ® 3.0 mg every 4 days + Doepezil + Memantine |
| | (Ebixa ® 10 mg) |
| PLENAXIS ® 100 mg given on days 1, 15 | PLENAXIS ® 100 mg given on days 1, 15 |
| and 28 and every 4 weeks afterward | and 28 and every 4 weeks afterward + Memantine |
| | (Ebixa ® 10 mg) |
| Antagon ™ 250 ug daily | Antagon ™ 250 ug daily + Doepezil + Memantine |
| | (Ebixa ® 10 mg) |
| Decapeptyl ® SR 3 mg 1 month | Decapeptyl ® SR 3 mg 1 month |
| subcutaenous and intramuscular injections | subcutaenous and intramuscular injections + Memantine |
| | (Ebixa ® 10 mg) |
| Decapeptyl ® SR 11.25 mg 3 month | Decapeptyl ® SR 11.25 mg 3 month |
| injection | injection + Memantine(Ebixa ® 10 mg) |

TABLE F
Combination of Memantine (NAMENDA ®) with selected GnRH-I analog
products for use in the method of the invention.

| | | Dosing Regimen |
|---|---|---|
| | Dosing Regimen | Memantine (NAMENDA ®) |
| | Memantine (NAMENDA ®) | Memantine |
| Dosing Regimen | Memantine (NAMENDA ® | (NAMENDA ® 10 mg |
| GnRH-I Analogs | 5 mg twice daily) | twice daily) |
| Lupron Depot ® 3.75 mg 1 | Lupron Depot ® 3.75 mg 1 | Lupron Depot ® 3.75 mg 1 |
| month injection | month injection + NAMENDA ® | month injection + NAMENDA ® |
| | 5 mg) | 10 mg |
| Lupron Depot ® 7.5 mg 1 | Lupron Depot ® 7.5 mg 1 month | Lupron Depot ® 7.5 mg 1 |
| month injection | injection + NAMENDA ® 5 mg) | month injection + NAMENDA ® |
| | | 10 mg |
| Lupron Depot-PED ® | Lupron Depot-PED ® 11.25 mg | Lupron Depot-PED ® |
| 11.25 mg 1 month | 1 month injection + NAMENDA ® | 11.25 mg 1 month |
| injection | 5 mg) | injection + NAMENDA ® |
| | | 10 mg |
| Lupron Depot-PED ® | Lupron Depot-PED ® 15 mg | Lupron Depot-PED ® |
| 15 mg injection | injection + NAMENDA ® 5 mg) | 15 mg injection + NAMENDA ® |
| | | 10 mg |
| Lupron Depot ® 22.5 mg 3 | Lupron Depot ® 22.5 mg 3 | Lupron Depot ® 22.5 mg 3 |
| month injection | month injection + NAMENDA ® | month injection + NAMENDA ® |
| | 5 mg) | 10 mg |
| Lupron Depot ® 30 mg 4 | Lupron Depot ® 30 mg 4 month | Lupron Depot ® 30 mg 4 |
| month injection | injection + NAMENDA ® 5 mg | month injection + NAMENDA ® |
| | | 10 mg |
| Lupron Depot ® | Lupron Depot ® multidose vials | Lupron Depot ® |
| multidose vials with | with 2.8 ml of 5 g/ml daily | multidose vials with |
| 2.8 ml of 5 g/ml daily | injections + NAMENDA ® 5 mg | 2.8 ml of 5 g/ml daily |
| injections | | injections + NAMENDA ® |
| | | 10 mg |
| Viadur ™ 72 mg 12 month | Viadur ™ 72 mg 12 month | Viadur ™ 72 mg 12 month |
| implantation | implantation + NAMENDA ® 5 mg | implantation + NAMENDA ® 10 mg |
| ELIGARD ® 7.5 mg 1 | ELIGARD ® 7.5 mg 1 month | ELIGARD ® 7.5 mg 1 |
| month injection | injection + NAMENDA ® 5 mg | month injection + NAMENDA ® |
| | | 10 mg |
| ELIGARD ® 22.5 mg 3 | ELIGARD ® 22.5 mg 3 month | ELIGARD ® 22.5 mg 3 |
| month injection | injection + NAMENDA ® 5 mg | month injection + NAMENDA ® |
| | | 10 mg |
| ELIGARD ® 30 mg 4 | ELIGARD ® 30 mg 4 month | ELIGARD ® 30 mg 4 |
| month injection | injection + NAMENDA ® 5 mg | month injection + NAMENDA ® |
| | | 10 mg |
| ELIGARD ® 45 mg 3 | ELIGARD ® 45 mg 3 month | ELIGARD ® 45 mg 3 |
| month injection | injection + NAMENDA ® 5 mg | month injection + NAMENDA ® |
| | | 10 mg |
| Zoladex ® 3.6 mg 1month | Zoladex ® 3.6 mg 1month + NAMENDA ® | Zoladex ® 3.6 mg |
| | 5 mg | 1month + NAMENDA ® |
| | | 10 mg |
| Synarel ® 200 | Synarel ® 200 micrograms twice | Synarel ® 200 |
| micrograms twice daily | daily + NAMENDA ® 5 mg | micrograms twice daily + NAMENDA ® |
| | | 10 mg |
| Synarel ® daily intranasal | Synarel ® daily intranasal | Synarel ® daily intranasal |
| dosings for children and | dosings for children and adults | dosings for children and |
| adults range from 200 ug | range from 200 ug to 1800 ug + NAMENDA ® | adults range from 200 ug |
| to 1800 ug | 5 mg | to 1800 ug + NAMENDA ® |
| | | 10 mg |
| TRELSTAR ® DEPOT | TRELSTAR ® DEPOT 3.75 mg | TRELSTAR ® DEPOT |
| 3.75 mg 1 month | 1 month + NAMENDA ® 5 mg | 3.75 mg 1 month + NAMENDA ® |
| | | 10 mg |
| TRELSTAR ® LA 11.25 mg | TRELSTAR ® LA 11.25 mg 12 | TRELSTAR ® LA 11.25 mg |
| 12 week injection | week injection + NAMENDA ® | 12 week injection + NAMENDA ® |
| | 5 mg | 10 mg |
| | | AChE inhibitors + Memantine |
| | AChE inhibitors + Memantine | Memantine |
| | (NAMENDA ® 5 mg twice | (NAMENDA ® 10 mg |
| GnRH-I Analogs | daily) | twice daily) |
| SUPPRELIN ® 200 ug/ml | SUPPRELIN ® 200 ug/ml daily | SUPPRELIN ® 200 ug/ml |
| daily injection | injection + NAMENDA ® 5 mg | daily injection + Memantine |
| | | (Ebixa ® |
| | | 10 mg) |
| SUPPRELIN ® 500 ug/ml | SUPPRELIN ® 500 ug/ml daily | SUPPRELIN ® 500 ug/ml |
| daily injection | injection + NAMENDA ® 5 mg | daily injection + NAMENDA ® |
| | | 10 mg) |
| SUPPRELIN ® | SUPPRELIN ® 1000 ug/ml daily | SUPPRELIN ® |
| 1000 ug/ml daily injection | injection + NAMENDA ® 5 mg | 1000 ug/ml daily injection + NAMENDA ® |
| | | 10 mg) |
| SUPPRELIN ® LA 50 mg | SUPPRELIN ® LA 50 mg 12 | SUPPRELIN ® LA 50 mg |
| 12 month implant | month implant + NAMENDA ® | 12 month implant + NAMENDA ® |
| | 5 mg | 10 mg |
| VANTAS ® 50 mg 12 | VANTAS ® 50 mg 12 month | VANTAS ® 50 mg 12 |
| month implant | implant + NAMENDA ® 5 mg | month implant + NAMENDA ® |
| | | 10 mg |
| SUPREFACT ® 6.3 mg 2 | SUPREFACT ® 6.3 mg 2 month | SUPREFACT ® 6.3 mg 2 |
| month implant | implant + NAMENDA ® 5 mg | month implant + NAMENDA ® |
| | | 10 mg |
| SUPREFACT ® 500 ug | SUPREFACT ® 500 ug every 8 | SUPREFACT ® 500 ug |
| every 8 hours for 7 days | hours for 7 days followed by | every 8 hours for 7 days |
| followed by 200 ug per | 200 ug per day + NAMENDA ® | followed by 200 ug per |
| day | 5 mg | day + NAMENDA ® 10 mg |
| SUPREFACT ® 9.5 mg 3 | SUPREFACT ® 9.5 mg 3 month | SUPREFACT ® 9.5 mg 3 |
| month implant | implant + NAMENDA ® 5 mg | month implant + NAMENDA ® |
| | | 10 mg |
| Cetrotide ® 0..25 mg daily | Cetrotide ® 0..25 mg daily + NAMENDA ® | Cetrotide ® 0..25 mg daily + NAMENDA ® |
| | 5 mg | 10 mg |
| Cetrotide ® 3.0 mg every 4 | Cetrotide ® 3.0 mg every 4 days + NAMENDA ® | Cetrotide ® 3.0 mg every 4 |
| days | 5 mg | days + NAMENDA ® 10 mg |
| PLENAXIS ® 100 mg | PLENAXIS ® 100 mg given on | PLENAXIS ® 100 mg |
| given on days 1, 15 and 28 | days 1, 15 and 28 and every 4 | given on days 1, 15 and 28 |
| and every 4 weeks | weeks afterward + NAMENDA ® | and every 4 weeks |
| afterward | 5 mg | afterward + NAMENDA ® |
| | | 10 mg |
| Antagon ™ 250 ug daily | Antagon ™ 250 ug daily + NAMENDA ® | Antagon ™ 250 ug daily + NAMENDA ® |
| | 5 mg | 10 mg |
| Decapeptyl ® SR 3 mg 1 | Decapeptyl ® SR 3 mg 1 month | Decapeptyl ® SR 3 mg 1 |
| month subcutaenous and | subcutaenous and intramuscular | month subcutaenous and |
| intramuscular injections | injections + NAMENDA ® 5 mg | intramuscular injections + NAMENDA ® |
| | | 10 mg |
| Decapeptyl ® SR 11.25 mg | Decapeptyl ® SR 11.25 mg 3 | Decapeptyl ® SR 11.25 mg |
| 3 month injection | month injection + NAMENDA ® | 3 month injection + NAMENDA ® |
| | 5 mg | 10 mg |

Using one or more GnRH-I analogs in combination with NMDA receptor antagonists is expected to have the net effect of reducing the number of neurons that die in AD brains. Combination therapy with one or more GnRH-I analogs, AChE inhibitors, and/or NMDA antagonists can prevent or slow neuronal death caused by aberrant cell cycling and glutamate toxicity and improve cholinergic neurotransmission.

In some embodiments of the invention, a three-way combination of agents may be administered to treat AD or MCI. For example, a three-way combination of one or more GnRH-I analogs, one or more AChE inhibitors, and one or more NMDA antagonists can be beneficial to patients. Utilizing the three different pathways of these agents in the treatment of AD can yield several advantages. For example, using a lower amount of a specific drug in a combination therapy can reduce the side effects from that drug while still yielding positive results from the combination therapy as a whole. Also, a dose of one drug that is smaller than the patient's usual dose can be administered while holding the other two drug doses constant. In such situations, increasing the first drug by a small amount can achieve significant benefits in treatment without appreciably increasing the side effects from that drug.

TABLE G
Combination of Memantine (Ebixa ® or NAMENDA ®) with selected GnRH-
I analog products and AChE inhibitor products for use in the method of the invention.

| | Dosing Regimen | | | |
|---|---|---|---|---|
| Dosing | AChE inhibitors + Memantine (Ebixa ® 10 mg or NAMENDA ® 5 or 10 mg) | | | |
| Regimen | Donepezil | Rivastigmine | Galantamine | Tetrahydroaminoacridine- |
| GnRH-I | (Aricept ®) + | (Exelon ®) + | (Reminyl ®) + | Tacrine (Cognex ®) + |
| Analogs | Memantine | Memantine | Memantine | Memantine |
| Lupron Depot ® | Lupron Depot ® | Lupron Depot ® | Lupron Depot ® | Lupron Depot ® 3.75 mg 1 |
| 3.75 mg 1 | 3.75 mg 1 | 3.75 mg 1 | 3.75 mg 1 | month injection + Tacrine + |
| month injection | month injection + | month injection + | month injection + | Memantine |
| | Donepezil + | Rivastigmine + | Galantamine + | |
| | Memantine | Memantine | Memantine | |
| Lupron Depot ® | Lupron Depot ® | Lupron Depot ® | Lupron Depot ® | Lupron Depot ® 7.5 mg 1 |
| 7.5 mg 1 month | 7.5 mg 1 month | 7.5 mg 1 month | 7.5 mg 1 month | month injection + Tacrine |
| injection | injection + | injection + | injection + | + Memantine |
| | Donepezil + | Rivastigmine + | Galantamine + | |
| | Memantine | Memantine | Memantine | |
| Lupron Depot- | Lupron Depot- | Lupron Depot- | Lupron Depot- | Lupron Depot-PED ® |
| PED ® 11.25 mg | PED ® 11.25 mg | PED ® 11.25 mg | PED ® 11.25 mg | 11.25 mg 1 month |
| 1 month | 1 month | 1 month | 1 month | injection + Tacrine + |
| injection | injection + | injection + | injection + | Memantine |
| | Donepezil + | Rivastigmine + | Galantamine + | |
| | Memantine | Memantine | Memantine | |
| Lupron Depot- | Lupron Depot- | Lupron Depot- | Lupron Depot- | Lupron Depot-PED ® |
| PED ® 15 mg | PED ® 15 mg | PED ® 15 mg | PED ® 15 mg | 15 mg injection + Tacrine |
| injection | injection + | injection + | injection + | + Memantine |
| | Donepezil + | Rivastigmine + | Galantamine + | |
| | Memantine | Memantine | Memantine | |
| Lupron Depot ® | Lupron Depot ® | Lupron Depot ® | Lupron Depot ® | Lupron Depot ® 22.5 mg 3 |
| 22.5 mg 3 | 22.5 mg 3 | 22.5 mg 3 | 22.5 mg 3 | month injection + Tacrine + |
| month injection | month injection + | month injection + | month injection + | Memantine |
| | Donepezil + | Rivastigmine + | Galantamine + | |
| | Memantine | Mamentine | Memantine | |
| Lupron Depot ® | Lupron Depot ® | Lupron Depot ® | Lupron Depot ® | Lupron Depot ® 30 mg 4 |
| 30 mg 4 month | 30 mg 4 month | 30 mg 4 month | 30 mg 4 month | month injection + Tacrine + |
| injection | injection + | injection + | injection + | Memantine |
| | Donepezil + | Rivastigmine + | Galantamine + | |
| | Memantine | Memantine | Memantine | |
| Lupron Depot ® | Lupron Depot ® | Lupron Depot ® | Lupron Depot ® | Lupron Depot ® |
| multidose vials | multidose vials | multidose vials | multidose vials | multidose vials with |
| with 2.8 ml of | with 2.8 ml of | with 2.8 ml of | with 2.8 ml of | 2.8 ml of 5 g/ml daily |
| 5 g/ml daily | 5 g/ml daily | 5 g/ml daily | 5 g/ml daily | injections + Tacrine + |
| injections | injections + | injections + | injections + | Memantine |
| | Doepezil + | Rivastigmine + | Galantamine + | |
| | Memantine | Memantine | Memantine | |
| Viadur ™ 72 mg | Viadur ™ 72 mg | Viadur ™ 72 mg | Viadur ™ 72 mg | Viadur ™ 72 mg 12 month |
| 12 month | 12 month | 12 month | 12 month | implantation + Tacrine + |
| implantation | implantation + | implantation + | implantation + | Memantine |
| | Donepezil + | Rivastigmine + | Galantamine + | |
| | Memantine | Memantine | Memantine | |
| ELIGARD ® | ELIGARD ® | ELIGARD ® | ELIGARD ® | ELIGARD ® 7.5 mg 1 |
| 7.5 mg 1 month | 7.5 mg 1 month | 7.5 mg 1 month | 7.5 mg 1 month | month injection + Tacrine + |
| injection | injection + | injection + | injection + | Memantine |
| | Donepezil + | Rivastigmine + | Galantamine + | |
| | Memantine | Memantine | Memantine | |
| ELIGARD ® | ELIGARD ® | ELIGARD ® | ELIGARD ® | ELIGARD ® 22.5 mg 3 |
| 22.5 mg 3 | 22.5 mg 3 | 22.5 mg 3 | 22.5 mg 3 | month injection + Tacrine + |
| month injection | month injection + | month injection + | month injection + | Memantine |
| | Donepezil + | Rivastigmine + | Galantamine + | |
| | Memantine | Mamentine | Memantine | |
| ELIGARD ® | ELIGARD ® | ELIGARD ® | ELIGARD ® | ELIGARD ® 30 mg 4 |
| 30 mg 4 month | 30 mg 4 month | 30 mg 4 month | 30 mg 4 month | month injection + Tacrine + |
| injection | injection + | injection+ | injection + | Memantine |
| | Donepezil + | Rivastigmine + | Galantamine + | |
| | Memantine | Memantine | Memantine | |
| ELIGARD ® | ELIGARD ® | ELIGARD ® | ELIGARD ® | ELIGARD ® 45 mg 3 |
| 45 mg 3 month | 45 mg 3 month | 45 mg 3 month | 45 mg 3 month | month injection + Tacrine + |
| injection | injection + | injection + | injection + | Memantine |
| | Donepezil + | Rivastigmine + | Galantamine + | |
| | Memantine | Memantine | Memantine | |
| Zoladex ® | Zoladex ® | Zoladex ® | Zoladex ® | Zoladex ® 3.6 mg 1month + |
| 3.6 mg 1month | 3.6 mg 1month + | 3.6 mg 1month + | 3.6 mg 1month + | Tacrine + Memantine |
| | Donepezil + | Rivastigmine + | Galantamine + | |
| | Memantine | Memantine | Memantine | |
| Synarel ® 200 | Synarel ® 200 | Synarel ® 200 | Synarel ® 200 | Synarel ® 200 |
| micrograms | micrograms | micrograms | micrograms | micrograms twice daily + |
| twice daily | twice daily + | twice daily + | twice daily + | Tacrine + Memantine |
| | Donepezil + | Rivastigmine + | Galantamine + | |
| | Memantine | Memantine | Memantine | |
| Synarel ® daily | Synarel ® daily | Synarel ® daily | Synarel ® daily | Synarel ® daily intranasal |
| intranasal | intranasal | intranasal | intranasal | dosings for children and |
| dosings for | dosings for | dosings for | dosings for | adults range from 200 ug |
| children and | children and | children and | children and | to 1800 ug + Tacrine + |
| adults range | adults range | adults range | adults range | Memantine |
| from 200 ug to | from 200 ug to | from 200 ug to | from 200 ug to | |
| 1800 ug | 1800 ug + | 1800 ug + | 1800 ug + | |
| | Donepezil + | Rivastigmine + | Galantamine + | |
| | Memantine | Memantine | Memantine | |
| TRELSTAR ® | TRELSTAR ® | TRELSTAR ® | TRELSTAR ® | TRELSTAR ® DEPOT |
| DEPOT | DEPOT | DEPOT | DEPOT | 3.75 mg 1 month + |
| 3.75 mg 1 | 3.75 mg 1 | 3.75 mg 1 | 3.75 mg 1 | Tacrine + Memantine |
| month | month + | month + | month + | |
| | Donepezil + | Rivastigmine + | Galantamine + | |
| | Memantine | Memantine | Memantine | |
| TRELSTAR ® | TRELSTAR ® | TRELSTAR ® | TRELSTAR ® | TRELSTAR ® LA 11.25 mg |
| LA 11.25 mg | LA 11.25 mg | LA 11.25 mg | LA 11.25 mg | 12 week injection + |
| 12 week | 12 week | 12 week | 12 week | Tacrine + Memantine |
| injection | injection + | injection + | injection + | |
| | Donepezil + | Rivastigmine + | Galantamine + | |
| | Memantine | Memantine | Memantine | |
| SUPPRELIN ® | SUPPRELIN ® | SUPPRELIN ® | SUPPRELIN ® | SUPPRELIN ® 200 ug/ml |
| 200 ug/ml daily | 200 ug/ml daily | 200 ug/ml daily | 200 ug/ml daily | daily injection + Tacrine + |
| injection | injection + | injection + | injection + | Memantine |
| | Donepezil + | Rivastigmine + | Galantamine + | |
| | Memantine | Memantine | Memantine | |
| SUPPRELIN ® | SUPPRELIN ® | SUPPRELIN ® | SUPPRELIN ® | SUPPRELIN ® 500 ug/ml |
| 500 ug/ml daily | 500 ug/ml daily | 500 ug/ml daily | 500 ug/ml daily | daily injection + Tacrine + |
| injection | injection + | injection + | injection + | Memantine |
| | Donepezil + | Rivastigmine + | Galantamine + | |
| | Memantine | Memantine | Memantine | |
| SUPPRELIN ® | SUPPRELIN ® | SUPPRELIN ® | SUPPRELIN ® | SUPPRELIN ® |
| 1000 ug/ml | 1000 ug/ml | 1000 ug/ml | 1000 ug/ml | 1000 ug/ml daily injection + |
| daily injection | daily injection + | daily injection + | daily injection + | Tacrine + Memantine |
| | Donepezil + | Rivastigmine + | Galantamine + | |
| | Memantine | Memantine | Memantine | |
| SUPPRELIN ® | SUPPRELIN ® | SUPPRELIN ® | SUPPRELIN ® | SUPPRELIN ® LA 50 mg |
| LA 50 mg 12 | LA 50 mg 12 | LA 50 mg 12 | LA 50 mg 12 | 12 month implant + |
| month implant | month implant + | month implant + | month implant + | Tacrine + Memantine |
| | Donepezil + | Rivastigmine + | Galantamine + | |
| | Memantine | Memantine | Memantine | |
| VANTAS ® | VANTAS ® | VANTAS ® | VANTAS ® | VANTAS ® 50 mg 12 |
| 50 mg 12 month | 50 mg 12 month | 50 mg 12 month | 50 mg 12 month | month implant + Tacrine + |
| implant | implant + | implant + | implant + | Memantine |
| | Donepezil + | Rivastigmine + | Galantamine + | |
| | Memantine | Memantine | Memantine | |
| SUPREFACT ® | SUPREFACT ® | SUPREFACT ® | SUPREFACT ® | SUPREFACT ® 6.3 mg 2 |
| 6.3 mg 2 month | 6.3 mg 2 month | 6.3 mg 2 month | 6.3 mg 2 month | month implant + Tacrine + |
| implant | implant + | implant + | implant + | Memantine |
| | Donepezil + | Rivastigmine + | Galantamine + | |
| | Memantine | Memantine | Memantine | |
| SUPREFACT ® | SUPREFACT ® | SUPREFACT ® | SUPREFACT ® | SUPREFACT ® 500 ug |
| 500 ug every 8 | 500 ug every 8 | 500 ug every 8 | 500 ug every 8 | every 8 hours for 7 days |
| hours for 7 | hours for 7 | hours for 7 | hours for 7 | followed by 200 ug per |
| days followed | days followed | days followed | days followed | day + Tacrine + |
| by 200 ug per | by 200 ug per | by 200 ug per | by 200 ug per | Memantine |
| day | day + | day + | day + | |
| | Donepezil + | Rivastigmine + | Galantamine + | |
| | Memantine | Memantine | Memantine | |
| SUPREFACT ® | SUPREFACT ® | SUPREFACT ® | SUPREFACT ® | SUPREFACT ® 9.5 mg 3 |
| 9.5 mg 3 month | 9.5 mg 3 month | 9.5 mg 3 month | 9.5 mg 3 month | month implant + Tacrine + |
| implant | implant + | implant + | implant + | Memantine |
| | Donepezil + | Rivastigmine + | Galantamine + | |
| | Memantine | Memantine | Memantine | |
| Cetrotide ® | Cetrotide ® | Cetrotide ® | Cetrotide ® | Cetrotide ® 0..25 mg daily |
| 0..25 mg daily | 0..25 mg daily + | 0..25 mg daily + | 0..25 mg daily + | + Tacrine + Memantine |
| | Donepezil + | Rivastigmine + | Galantamine + | |
| | Memantine | Memantine | Memantine | |
| Cetrotide ® | Cetrotide ® | Cetrotide ® | Cetrotide ® | Cetrotide ® 3.0 mg every 4 |
| 3.0 mg every 4 | 3.0 mg every 4 | 3.0 mg every 4 | 3.0 mg every 4 | days + Tacrine + |
| days | days + | days + | days + | Memantine |
| | Donepezil + | Rivastigmine + | Galantamine + | |
| | Memantine | Memantine | Memantine | |
| PLENAXIS ® | PLENAXIS ® | PLENAXIS ® | PLENAXIS ® | PLENAXIS ® 100 mg |
| 100 mg given | 100 mg given | 100 mg given | 100 mg given | given on days 1, 15 and |
| on days 1, 15 | on days 1, 15 | on days 1, 15 | on days 1, 15 | 28 and every 4 weeks |
| and 28 and | and 28 and | and 28 and | and 28 and | afterward + Tacrine + |
| every 4 weeks | every 4 weeks | every 4 weeks | every 4 weeks | Memantine |
| afterward | afterward + | afterward + | afterward + | |
| | Donepezil + | Rivastigmine + | Galantamine + | |
| | Memantine | Memantine | Memantine | |
| Antagon ™ | Antagon ™ | Antagon ™ | Antagon ™ | Antagon ™ 250 ug daily + |
| 250 ug daily | 250 ug daily + | 250 ug daily + | 250 ug daily + | Tacrine + Memantine |
| | Donepezil + | Rivastigmine + | Galantamine + | |
| | Memantine | Memantine | Memantine | |
| Decapeptyl ® | Decapeptyl ® | Decapeptyl ® | Decapeptyl ® | Decapeptyl ® SR 3 mg 1 |
| SR 3 mg 1 | SR 3 mg 1 | SR 3 mg 1 | SR 3 mg 1 | month subcutaenous and |
| month | month | month | month | intramuscular injections + |
| subcutaenous | subcutaenous | subcutaenous | subcutaenous | Tacrine + Memantine |
| and | and | and | and | |
| intramuscular | intramuscular | intramuscular | intramuscular | |
| injections | injections + | injections + | injections + | |
| | Donepezil + | Rivastigmine + | Galantamine + | |
| | Memantine | Memantine | Memantine | |
| Decapeptyl ® | Decapeptyl ® | Decapeptyl ® | Decapeptyl ® | Decapeptyl ® SR |
| SR 11.25 mg 3 | SR 11.25 mg 3 | SR 11.25 mg 3 | SR 11.25 mg 3 | 11.25 mg 3 month |
| month | month | month | month | injection + Tacrine + |
| injection | injection + | injection + | injection + | Memantine |
| | Donepezil + | Rivastigmine + | Galantamine + | |
| | Memantine | Memantine | Memantine | |

In accordance with embodiments of the present invention, decreased blood and tissue levels, production, function, and activity of FSH and LH, along with AChE inhibition at neuronal synapses, prevents aborted cell cycling of terminally differentiated neurons and elevates the levels of acetylcholine in neuronal synapses of the basal forebrain, amygdala, hippocampus, and entorhinal cortex, thus treating, mitigating, slowing the progression of, and/or preventing AD or MCI.

In other embodiments of the invention, decreased blood and tissue levels, production, function, and activity of FSH and LH, along with decreased glutamate-stimulated excitotoxicity, prevents aborted cell cycling of terminally differentiated neurons and prevents neuronal death due to glutamate-induced neuronal excitotoxicity, thus treating, mitigating, slowing the progression of, and/or preventing AD or MCI.

In other embodiments of the invention, decreased blood and tissue levels, production, function, and activity of FSH and LH, along with AChE inhibition at neuronal synapses and decreased glutamate-stimulated neuronal excitotoxicity, prevents aborted cell cycling of terminally differentiated neurons, elevates the levels of acetylcholine in neuronal synapses of the basal forebrain, amygdala, hippocampus, and entorhinal cortex, and prevents neuronal death due to glutamate-induced neuronal excitotoxicity, thus treating, mitigating, slowing the progression of, and/or preventing AD.

### EXAMPLES

### Example 1

### Exemplary Method of Treatment of AD or MCI in Patients Using a Commercially-Available, Injectable, Time-Release Suspension of Leuprolide in Polymer Microspheres According to an Embodiment of the Invention

The following description of Example 1's compositions and procedures for administration are based on publicly available materials. (See, e.g., http://products.sanofi-aventis.us/eligard/eligard_{—}225.html.) The results described for treatments of AD are results that are expected in view of the publicly available materials and this specification.

The commercially-available product, ELIGARD® 7.5 mg is a sterile polymeric matrix formulation of leuprolide acetate used for subcutaneous injection. It is designed to deliver 7.5 mg of leuprolide acetate at a controlled rate over a one month therapeutic period.

Leuprolide acetate is the acetate salt-form of a synthetic nonapeptide analog of naturally occurring gonadotropin releasing hormone (GnRH) that, when given continuously, inhibits pituitary gonadotropin secretion and suppresses testicular and ovarian steroidogenesis. The analog possesses greater potency than the natural hormone. The chemical name is 5-oxo-L-prolyl-L-histidyl-L-tryptophyl-L-seryl-L-tyrosyl-D-leucyl-

L-leucyl-L-arginyl-N-ethyl-L-prolinamide acetate (salt) with the following structural formula:

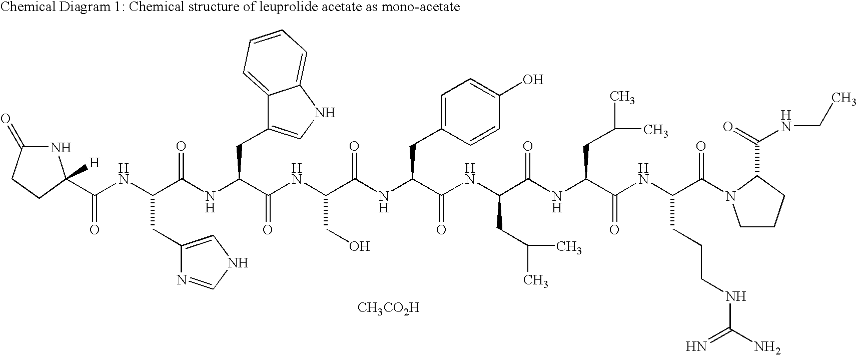

A clinician follows the directions on the ELIGARD® package insert to prepare the drug use for treatment of a patient with AD or MCI, which is similar to the procedure used in the treatment for a use known in the art, summarized as follows. ELIGARD® 7.5 mg is prefilled and supplied in two separate, sterile syringes whose contents are mixed immediately prior to administration. The two syringes are joined and the single dose product is mixed until it is homogenous. ELIGARD® 7.5 mg is administered subcutaneously where it forms a solid drug delivery depot.

One syringe contains the ATRIGEL® Delivery System and the other contains leuprolide acetate. The ATRIGEL® Delivery System is a polymeric (non-gelatin containing) delivery system consisting of a biodegradable poly(DL-lactide-co-glycolide) (PLGH) polymer formulation dissolved in a biocompatible solvent, N-methyl-2-pyrrolidone (NMP). PLGH is a co-polymer with a 50:50 molar ratio of DL-lactide to glycolide containing carboxyl end groups. The second syringe contains leuprolide acetate, and the constituted product is designed to deliver 7.5 mg of leuprolide acetate at the time of subcutaneous injection.

ELIGARD® 7.5 mg delivers 7.5 mg of leuprolide acetate (equivalent to approximately 7.0 mg leuprolide free base) dissolved in 160 mg N-methyl-2-pyrrolidone and 82.5 mg poly(DL-lactide-coglycolide). The approximate weight of the administered formulation is 250 mg. A clinician or trained person injects the syringe contents into a person having (or suspected of having) AD or MCI to achieve the desired effect. In some cases, it will be desirable for a clinician to inject a patient with two or three injections of ELIGARD® 7.5 mg at about the same time, or within a week of each other to achieve the desired effect of treating or slowing the decline of a person's cognitive or mental abilities. This multiple-dose treatment may be especially important when a patient is overweight or obese.

Following the first dose of ELIGARD® 7.5 mg, mean serum gonadotropin hormone concentrations and mean serum sex steroid concentrations increase, then fall to low levels within three weeks. Continued monthly treatments with ELIGARD® 7.5 mg maintains the hormone suppression throughout the study and provides beneficial treatment effects to a patient with Alzheimer's disease or MCI, as determined using known methods for scoring a patient's cognitive abilities and mental functions such as memory loss, e.g., the ADAS-cog test, the ADCS-CGIC test, the ADCS-ADL test, and/or other clinically-proven tests found useful for such determinations.

The treatment is expected to be useful to slow the progression of MCI or AD mental deficits and/or cognitive deficits in a patient. Successful treatment can be measured by comparing a patient's rate of cognitive decline over a time period of 2 months, or 3 months, or another time period, compared to the rates of decline of mental and cognitive abilities which have been observed in groups of untreated patients with AD or MCI over the same time periods.

### Example 2

### Exemplary Method of Treatment of AD or MCI in Patients Using an Injectable, Time-Release Suspension of Triptorelin in a Commercially-Available, Polymer Granule Formulation According to Various Embodiments of the Invention

The following description of Example 2's compositions and procedures for administration are based on publicly available materials. (See, e.g., http://pi.watson.com/prescribing_info.asp?type=pi&product_group=1314.) The results described for treatments of AD are results that are expected in view of the publicly available materials and this specification.

TRELSTAR® LA contains a pamoate salt of triptorelin. Triptorelin is a synthetic decapeptide agonist analog of GnRH-I with greater potency than the naturally occurring GnRH. The chemical name of triptorelin pamoate is 5-oxo-L-prolyl-L-histidyl-L-tryptophyl-L-seryl-L-tyrosyl-D-tryptophyl-L-leucyl-L-arginyl-L-prolylglycine amide (pamoate salt); the empirical formula is C₆₄H₈₂N₁₈O₁₃.C₂₃H₁₆O₆, and the molecular weight is 1699.9. The structure of the chemical is shown below in Chemical Diagram 2.

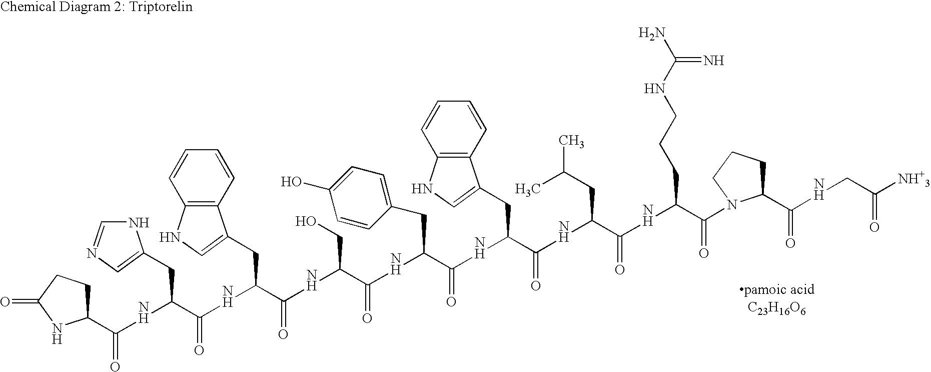

TRELSTAR® LA is a sterile, lyophilized, biodegradable microgranule formulation supplied as a single-dose vial containing triptorelin pamoate (11.25 mg as the peptide base), 145 mg poly-d,l-lactide-co-glycolide, 85 mg mannitol, USP, 30 mg carboxymethylcellulose sodium, USP, 2 mg polysorbate 80, NF. When 2 mL sterile water for injection is added to the vial containing TRELSTAR® LA and mixed, a suspension is formed which is intended as an intramuscular injection to be administered every 84 days (i.e., every 12 weeks). TRELSTAR® LA is available in 2 packaging configurations: (a) TRELSTAR® LA vial alone, or (b) TRELSTAR® LA vial plus a separate pre-filled syringe that contains sterile water for injection, USP, 2 mL, pH 6 to 8.5 (Clip‘n’Ject®).

Triptorelin is a potent inhibitor of gonadotropin secretion when administered continuously and in therapeutic doses. Following the first administration of a single intramuscular (IM) injection of TRELSTAR® LA, there is a transient surge in circulating levels of luteinizing hormone (LH), follicle-stimulating hormone (FSH), testosterone, and estradiol. After chronic and continuous administration, usually 2 to 4 weeks after initiation of therapy, a sustained decrease in LH and FSH secretion and marked reduction of testicular and ovarian steroidogenesis is expected to be observed. In men, a reduction of serum testosterone concentration to a level typically seen in surgically castrated men is obtained. Consequently, tissues and functions that depend on these hormones for maintenance often become quiescent.

A clinician follows the directions on the TRELSTAR® LA package insert to prepare the drug dose for treatment of a patient with AD or MCI, which is similar to the procedure used in the treatment for a use known in the art. It should be noted that triptorelin is not orally active.

A clinician or trained person injects the syringe contents (intramuscular injection) into a person with AD or MCI, or one who is suspected of having AD or MCI, to achieve the desired effect. In some cases, it will be desirable for a clinician to inject a patient with two or three injections of TRELSTAR® LA at about the same time, or within a week of each other to achieve the desired effect of treating or slowing the decline of a person's cognitive or mental abilities. This multiple-dose treatment may be especially important when a patient is overweight or obese, or for example, as in the case where the dosage determined in Example 3 is greater than the amount of active drug in one injectable dose.

Following the first single intramuscular (IM) injection of TRELSTAR® LA dose (11.25 mg of active material), mean serum gonadotropin hormone concentrations and mean serum sex steroid concentrations transiently increase, then fall to low levels within three weeks. Continued treatments with TRELSTAR® LA at 12-week intervals maintains the hormone suppression throughout the study or treatment period, and provides beneficial treatment effects to a patient with mild cognitive impairment or Alzheimer's disease, as determined using known methods for scoring of a patient's cognitive abilities and mental functions such as memory loss by using the ADAS-cog test, the ADCS-CGIC test, the ADCS-ADL test, and/or other clinically-proven tests found useful for such determinations and monitoring.

The treatment is expected to be useful to slow the progression of MCI or AD mental deficits and/or cognitive deficits in a patient. Successful treatment can be measured by comparing a patient's rate of cognitive decline over a time period of 2 months, or 3 months, or another time period, compared to the rates of decline of mental and cognitive abilities which have been observed in groups of untreated patients (or patients treated with a single drug) with AD or MCI over the same time periods.

### Examples 3A-3G

### Dosage Ranges of GnRH-I Analogs Useful for Treating or Slowing the Rate of Cognitive Decline in AD Patients and Patients with MCI

The following description of Example 3A-3G's compositions and procedures for administration are based on publicly available materials. (See, e.g., http://pi.watson.com/prescribing_info.asp?type=pi&product_group=1314, http://www.eligard.com/hcp/pi/pi.asp, http://www.tap.com/pi.asp, http://www.viadur.com/, http://www.trelstar.com/about/pres_information.asp). The results described for treatments of AD are results that are expected in view of the publicly available materials and this specification.

3A) For a GnRH-I analog which has a molecular weight of between 1000 g/mol and 1200 g/mol for the peptide free-base portion (the active material) of a GnRH-I analog salt, the initial dosage can be adjusted to be between 0.0015 mg/patient-lb/day and 0.0025 mg/patient-lb/day, where the mg unit represents the drug amount (as the free base form) introduced, the patient-lb unit represents the weight of the patient in pounds, and the day unit refers to the number of days that a therapeutic dose of analog is expected to be present in the patient, based upon a time-release formulation, for an initial beneficial result. The initial dosage may also be a different amount, such as a range of between 0.003 mg/patient-lb/day and 0.005 mg/patient-lb/day.

3B) For a GnRH-I analog which has a molecular weight of between about 1200 g/mol and 1400 g/mol for the peptide free-base portion (the active material) of the GnRH-I analog salt, the initial dosage of peptide (calculated as the analog free base portion) can be adjusted to be between 0.0017 mg/patient-lb/day and 0.003 mg/patient-lb/day, or between 0.0035 mg/patient-lb/day and 0.006 mg/patient-lb/day. For example, if the analog is leuprolide, the molecular weight of its acetate salt is about 1270 g/mol, and that of the free base is about 1209 g/mol. For a patient who weighs 130 lbs and who is given an injection of leuprolide acetate that lasts about 30 days, the amount of leuprolide (calculated as peptide free base) used in an injected dose should be between 0.0017 mg×130 patient-lb×30 day and 0.003 mg×130 patient-lb×30 day. In other words, the amount of free base leuprolide peptide to be injected into the patient is calculated to be between 6.6 mg and 11.7 mg of leuprolide free base for the injection which is released over about 30 days. In this case, an injection of ELIGARD® 7.5 mg would be useful as a starting point for beginning the therapy with 30-day therapy intervals, as the dosage of active compound in one injection is about 7.0 mg of leuprolide (as free base) in the 7.5 mg of leuprolide acetate. Since 7.0 mg of free base leuprolide peptide analog falls within the appropriate range of 6.6 mg and 11.7 mg of active peptide material (as the free base) for the initial dose above, one injection of ELIGARD® per month is expected to suffice for this patient.

3C) For a GnRH-I analog that has a molecular weight of between about 1400 g/mol and 1600 g/mol for the peptide free-base portion (the active material) of the GnRH-I analog salt (or free base hydrate, for example), the initial dosage of analog can be adjusted to be between 0.002 mg/patient-lb/day and 0.0035 mg/patient-lb/day to give beneficial results.

3D) For a GnRH-I analog that has a molecular weight of between about 1600 g/mol and 1800 g/mol for the peptide free-base portion (the active material) of a GnRH-I analog salt, the initial dosage of analog can be adjusted to be between 0.0022 mg/patient-lb/day and 0.0039 mg/patient-lb/day to give beneficial results. For example, if the analog used in the invention is a polyethyleneglycol ether derivative of triptorelin which has an average molecular weight for the peptide free-base portion (the active material) of the GnRH-I analog of about 1700 g/mol, then the initial dosage of analog (calculated as mg of the free base present in the dose of any useful pharmaceutical salt form) can be adjusted to be between 0.0022 mg/patient-lb/day and 0.0039 mg/patient-lb/day to give beneficial results. For treatment of a 125-pound patient using a 12-month, time-release insert (similar to that used in a Viadur™ implant) that contains this analog, the amount of analog free base (calculated as peptide free base) used in the insert should be between 0.0017 mg×125 patient-lb×365 day and 0.003 mg×125 patient-lb×365 day. In other words, for a beneficial effect, the amount of this free base PEG-peptide (either as the free base, or in a suitable salt form such as a hydrochloride, hydrobromide, acetate or benzoate) to be implanted into the patient is calculated to be between 100 mg and 177 mg of the free base PEG-peptide analog which is released from the time-release formulation (after an initial burst of material during the first week or so) at a relatively even rate over about a year.

3E) For a patient weighing 110 lbs, the amount of the above mentioned polyethyleneglycol ether derivative of triptorelin (free base MW=1700 g/mol) desired for use in a slow-release formulation of biodegradable polymer microspheres that provides about 12 weeks or 84 days of relatively even drug release, the beneficial amount of the above mentioned polyethyleneglycol ether derivative of triptorelin (calculated as free base peptide analog) used in an injected dose for this patient should be between 0.0022 mg×110 patient-lb×84 day and 0.0039 mg×110 patient-lb×84 day or between 0.004 mg×110 patient-lb×84 day and 0.008 mg x patient-lb×84 day. In other words, between 20.3 mg and 36.0 mg should be the weight of the active free base triptorelin polyethyleneglycol ether derivative in the injected time-release formulation which lasts about 12 weeks, in order to see beneficial effects in delay or slowing of the rate of cognitive decline, relative to what is expected for rates of decline known in the art.

3F) When using triptorelin pamoate, the molecular weight of the pamoate salt is about 1700 g/mol. However, the free base peptide has a molecular weight of about 1311 g/mol. Therefore, if a patient weighs 110 lbs and is given an injection of a slow-release formulation of biodegradable polymer microspheres containing triptorelin pamoate that provides about 12 weeks or 84 days of relatively even drug release, then the beneficial amount of the triptorelin (calculated as the free base) used in an injected dose should be calculated using the ranges in Example 3B, not the ranges in Example 3E. The range of active free base-useful in such a formulation would be calculated to be between between 0.0017 mg/patient-lb/day and 0.003 mg/patient-lb/day. Or, for this example, between 0.0017 mg×110 patient-lb×84 day and 0.003 mg×110 patient-lb×84 day, that is between 15.7 mg and 27.7 mg of triptorelin free base peptide as the active component in the triptorelin pamoate in the injection which lasts about 12 weeks. In this case, 2 injections of the commercially-available product, TRELSTAR® LA, would supply an adequate amount of active material. Each injection contains 11.25 mg of the active material, and the 22.5 mg supplied by the double injection would be useful as a starting point for therapy, as the dosage of active compound using two injections falls within the appropriate range for initial dosage above.

According to some embodiments of the invention, a combination therapy method uses a GnRH-I analog regimen together with an AChEI drug regimen to produce added benefits in treated patients. Further examples are described below.

### Examples 4A and 4B

### Combination Therapy Approaches Using an AChEI Drug Regimen Together with a GnRH-I Analog Regimen, According to Various Embodiments of the Invention

4A) It is useful to treat a patient or a group of patients with both a GnRH-I analog, such as is found in ELIGARD® 7.5 mg, TRELSTAR® and TRELSTAR® LA, Viadur, Zoladex, Synarel® formulations, Lupron Depot® formulations, SUPPRELIN® formulations, VANTAS, SUPREFACT formulations, Cetrotide® formulations, Plenaxis®, Antagon™, and Decapeptyl® formulations to lower gonadotropins and sex steroids, in combination with the concomitant use of an AChE inhibitor drug therapy regimen over time to slow or delay the progressive deterioration of cognitive and mental functions in patients with AD or MCI. A clinician first determines if a patient with AD or MCI (or who is suspected of having AD or MCI by the clinician), responds well to a certain AChEI treatment, such as found by using Aricept®, Exelon®, Reminyl®, Cognex® or another AChE inhibitor as provided using techniques described in their respective product inserts. This initial determination can be made over the course of about two weeks to two months. Following the initial determination of a suitable AChEI drug and dose level for a patient or group of patients, the clinician begins a second therapy regimen with a GnRH-I analog such as leuprolide, triptorelin, histrelin, buserelin, cetrorelix, abarelix, ganirelix, nafarelin, or goserelin. Beneficial results from the combination of therapies can be determined by comparing the rates of decline of ADAS-cog scores for a patient or group of patients on dual therapy over time with the expected rates of decline known in the art for patients on a AChEI mono-therapy alone, or compared to patients on a GnRH-I mono-therapy alone, or compared to those who are on no drug therapy treatment. Clinical rates of decline are known in the art for treatment with Aricept®, Exelon®, Reminyl®, or Cognex® and can be found in various sources such as the Physician's Desk Reference or in product inserts.

4B) Alternatively, a patient or group of patients who have AD or MCI can be treated with a GnRH-I analog first at an initial dose level such as determined by the methods disclosed in the examples above for an initial period such as a week, two weeks, three weeks or a month or two months, to determine if the initial dose of a drug is adequate to produce a beneficial cognitive effect in that patient or group of patients, or to reduce the rate of cognitive or mental decline, which may be judged by a physician, a caretaker, or in some cases of mild AD or mild cognitive impairment, by the patient. Following the first determination period, the clinician can investigate if the patient or group of patients can benefit from a larger dose of the GnRH-I analog. During this second investigative period, the clinician uses twice the dose of GnRH-I analog which was used in the initial investigative period for the patient or patient group, and the effects of the increased dose on the cognitive abilities or rates of cognitive and mental decline are again determined after a period of two weeks, three weeks, a month, or two months. If a determination is made that the higher dose has been more helpful to the patient or group of patients than the initial dose, then this process is continued for a third investigative period wherein the drug dosage is again doubled (to four-fold over the initial dose). This process of dose-effect determination by dosage doubling can be continued until the clinician, caretaker, or patient determine that no further benefit, or very little added benefit is derived from an increased dose of GnRH-I analog, or until the clinician determines that one or more side effects from the increased drug dosage outweigh the benefits of the increased dose to the patient.

It should be noted that the majority of clinical trials measuring the effects of AChEIs in mild to moderate Alzheimer's disease patients have been of 12-24 weeks in duration (Physicians' Desk Reference, 59th edition, 2005, pp. 1197-1200, 1736-1741, 2304-2311), but there are at least two studies in which data was obtained over a 12-month period. (Wilcock G, Howe I, Coles H, Lilienfeld S, Truyen L, Zhu Y, Bullock R, Members of the GAL-GBR-2 Study Group. A long-term comparison of galantamine and donepezil in the treatment of Alzheimer's disease. Drugs Aging 20:777-789, 2003; and Feldman H H, Baelen B V, Kavanagh S M, Torfs K E L. Cognition, function, and caregiving time patterns in patients with mild-to-moderate Alzheimer disease. Alzheimer Disease and Associated Disorders 19:29-36, 2005.)

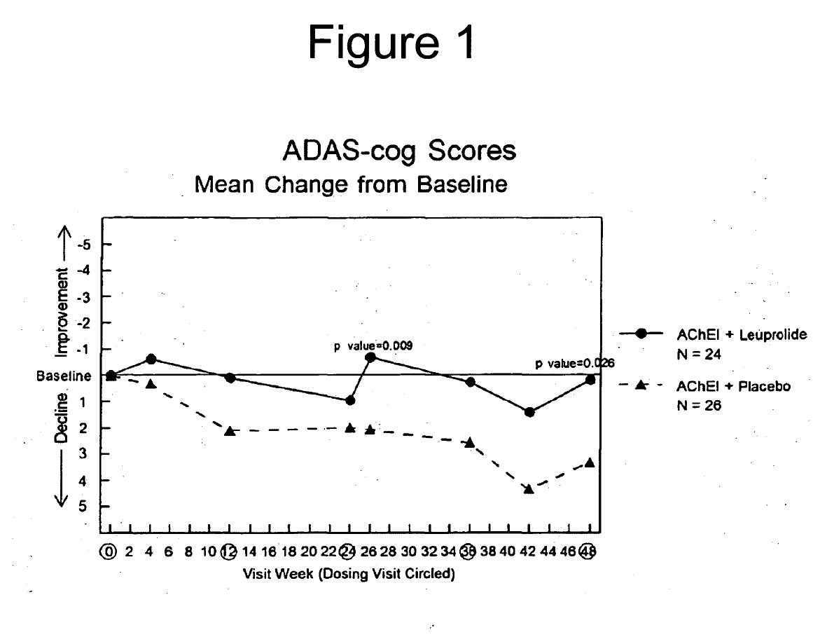

The cognition data presented in FIGS. 1, 2, 3, and 4 are from a clinical trial of 48 weeks duration. Those of ordinary skill in the art may expect this data to better compare to data available from 12-month trials than to data from trials of shorter periods. The expected or typical cognitive decline in mild to moderate Alzheimer's patients over a 12 month time period has been described in two 12-month trials, described below.

Wilcock et al. described a clinical trial comparing two AChEIs that was conducted over a one year period. Galantamine patients demonstrated a decline of 2.22±0.77 points compared with a decline of 3.43±0.8 for donepezil patients after 52 weeks of treatment, using the ADAS-cog outcome measure.

In another study, placebo data were pooled from two 1-year, randomized, double-blind, placebo-controlled trials of a candidate glutamate antagonist (sabeluzole) to assess the typical decline in groups of patients <85 years of age (see Feldman H H, Baelen B V, Kavanagh S M, Torfs K E L. Cognition, function, and caregiving time patterns in patients with mild-to-moderate Alzheimer disease. Alzheimer Disease and Associated Disorders 19:29-36, 2005). These combined studies represented 331 observed cases of human patients treated with placebo only. The mean [±SD] change from baseline in ADAS-cog score over 12 months for untreated Alzheimer's patients (age range 72.7±0.5) was 5.6±7.3, with a greater decline in moderate patients compared to mild patients.

Together, these studies demonstrate that a typical 1-year “placebo” decline on the ADAS-cog scale in Alzheimer's patients is 5.6, while the typical AChEI-treated 1-year decline is expected to be between 2.22 and 3.43.

From the clinical trial data presented in this specification, female patients in the mild-to-moderate AD category receiving AChEIs declined an average of 3.30 points over 48 weeks. This result is comparable to those provided above for the cognitive decline found in the other AChEI trials. By comparison, female patients in the mild-to-moderate AD category receiving 22.5 mg leuprolide acetate declined 4.68 points over 48 weeks, which is about one point of cognitive decline less than that expected from the literature data for placebo only-treated patients. Based on the above referenced, published data from other trials, patients receiving AChEIs declined between 2.22 and 3.43 ADAS-cog points (mean=2.82) over 1 year. Both the AChEI-only females (ADAS-cog deterioration=3.30) and the leuprolide-only females (ADAS-cog deterioration=4.68) performed better at the end of our study than a typical placebo population (ADAS-cog deterioration=5.6 decline) did at the end-of-year studies. In contrast, females receiving the combination of AChEIs and leuprolide acetate unexpectedly and surprisingly did much better than the placebo group or either drug group and showed only a 0.18 point cognitive decline at 48 weeks. Conversion of ADAS-cog scores to percent improvement over placebo results in the following outcomes:

If a cognitive decline of 5.6 ADAS-cog points is considered to represent the 100% expected normal decline for mild-to-moderate AD patients in a year, then the following conclusions can be made:

Patients on AChE inhibitors had a smaller cognitive decline (only 59%) than the expected no-treatment results in the literature (100% decline=5.6 points). Patients on leuprolide had a cognitive decline (only 84%) of the expected, no-treatment result. An expected outcome from additivity alone would have been 100%−(41%+16%)=43% decline of the cognition score obtained from the untreated group of patients after a year.

In contrast, however, patients on AChE inhibitors plus leuprolide had overall a cognitive decline of only 3% of the expected (i.e., no-treatment cognitive decline) result (which is not even in the same order of magnitude as the “expected cognitive decline” from an additive effect). This shows that an unexpected, synergistic result has occurred in these patients.

Another method of explanation may serve to further clarify these effects. If the change from baseline over 48 weeks is considered, then the decline (worsening) per week can be calculated:

Typical placebo decline of 5.6 equals an 11.6% worsening per week.

AChEI-only females declined 3.30 points over 48 weeks for a 6.9% worsening per week.

Leuprolide-only females declined 4.68 points over 48 weeks for a 9.8% worsening per week.

AChEI+leuprolide females declined 0.18 points over 48 weeks for a 0.375% worsening per week.

When the published results of other studies are compared to the data achieved using an embodiment of the combination of the present invention, the 0.375% weekly decline for the combination therapy group would not have been predicted. The benefit of adding the drugs together may have achieved on average an 8.35% weekly decline (based on the average expected decline of adding together a 6.9% decline in AChEI-only females and a 9.8% decline in leuprolide-only females: 6.9%+9.8%=16.7%/2=8.35%).

There are a number of ways to determine if a synergy of actions is produced from a combination of drug therapies. Examples of methods contemplated for use to determine synergism of drug effects are provided in Examples 5A and SB.

### Example 5A

### Exemplary Method for Determination of Synergism of Drug Effects According to Various Embodiments of the Invention

A therapeutic dose of a pharmaceutical composition comprising a GnRH-I analog for the treatment of AD or MCI is found using one of the methods known for determination of pharmaceutical effects, such as is used, for example, to determine efficacy of leuprolide in Table 3. The effect of using the GnRH-I analog in a patient or a group of patients with AD or MCI is noted over a time interval, such as over 2 months or 3 months, and is compared to what is expected in the art as a normal rate of decline for untreated AD or MCI patients, as measured by tests known in the art for measuring cognition changes, memory changes, or the like. As a second step, the GnRH-I analog drug dosage is doubled relative to the first dose, and the effect on the rate of decline from the doubled-dose is again noted over the same or a similar time period as for the first dose. The efficacies of the two dosage levels are compared to one another and the benefits and side effects found using the higher dose are noted. The next step is the addition of a pharmaceutically-active dose of an AChEI drug to the therapy regimen of the patient or patients, which is followed by the monitoring of the combined effects of the drugs over the same or a similar time interval as in the first step (e.g., 2 months or 3 months). Again, the rate of decline in test scores is measured and is compared to the rate of decline expected from literature examples of untreated patients with AD or MCI, and with patients treated with the AChEI drug. It is known in the art that the efficacious amounts of AChEI drugs that can be used in a patient or patients are limited to fairly narrow ranges due to the side effects produced by these materials. These side effects become more apparent at higher doses of each AChEI used. In other words, it is known in the art that the beneficial effects of drugs in the AChEI classification are limited by their respective dosage-ceilings caused by side effects. In general, doubling of dosages for AChE inhibitors is known to produce small, incremental gains for patients, not a doubling of efficacy. Therefore, if the concomitant use of a GnRH-I analog at the original efficacious dosage level found in the first step above plus the use of an AChEI pharmaceutical used at an initial efficacious level provides more benefit to a patient or patients, in the opinion of a qualified clinician, than the effect found from a doubled dose of either the GnRH-I analog used alone as above, or of a doubled dose of an AChEI pharmaceutical used alone as known from clinical trial literature, then it can be considered that the dual-therapy combination of the GnRH-I analog and the AChEI drug acts synergistically to provide significantly greater benefit to the patient or patients who are under treatment for AD or MCI.

Alternatively, if a therapeutic outcome of a combination of these drug regimens is determined to be of the same or similar benefit level to a patient or group of patients as on either the AChEI treatment alone or the GnRH-I analog therapy alone, but the side effects produced by the combination of drug regimens is decreased relative to the side effects produced by either of the drugs used alone, in the opinion of a qualified clinician, then this result is also a synergistic result, as the patient or group of patients benefits significantly and/or unexpectedly from the combination of drug regimens through a decrease in the level of side effects normally expected for a combination.

Synergy of drug effects can also be determined using a method such as one disclosed by Berenbaum (Berenbaum, M C. What is Synergy? Pharm. Reviews 41: 93-141, 1989). To determine if synergy arises from combination therapy treatments using a GnRH-I analog therapy and an AChE inhibitor therapy in a patient or a group of patients, a study can undertaken which is exemplified in Example 5B.

### Example 5B

### Determination of Synergy in Combination Therapy Using a GnRH-I Analog and a AChE Inhibitor Drug for the Treatment or Prevention of AD or MCI in a Patient or Patients or for the Slowing of the Progression of Cognitive or Mental Decline in a Patient or Patients, According to Various Embodiments of the Invention

When the concomitant use of two drug regimens for AD or MCI therapy or prevention results in a rate of decline of a cognitive score over time that is less than the decline scores expected over time for each of the mono-therapy treatments used alone, then three possibilities arise for labeling the extent of the outcome or benefit to the patient or group of patients. The outcome of the combination of treatments, according to Berenbaum and others, can be considered as synergistic, (somewhat) antagonistic, or indicative of non-interaction between the treatment regimens. The appropriate label for the interaction can be applied to the result of the combination therapy using the approach developed by Berenbaum, paraphrased as follows (see Berenbaum, page 96):
“In describing these [approaches], the following symbols will be used for combinations of two agents A and B . . . . The combination [dose of agents] is termed (da,db) where da and db are the doses (or concentrations if appropriate) of A and B, respectively. Effect is treated as a mathematical function E; thus, E(dₐ,d_{b}) or, where an explicit algebraic function can be used, ƒ(dₐ,d_{b}) is the effect of the combination . . . Dₐ and D_{b} are the doses of A and B separately that are isoeffective with the combination . . . .”
“The equation for the zero interaction line [addition of, rather than synergy of, combined effects] for two agents is [equation (1) of Page 96]:

$$\frac{{d}_{a}}{{D}_{\underline{a}}} + \frac{{d}_{b}}{{D}_{\underline{b}}} = 1 \qquad ( 1 ) ”$$

Berenbaum moves on to describe synergy (on page 97), presenting an inequality that describes when the synergy of effects of two agents (or the “interaction” between two agents in a system) is present:
“When agents in combination are more effective than expected from their dose-response curves (synergy), smaller amounts are needed to produce the effect under consideration, i.e., dₐ and/or d_{b} are reduced, while Dₐ and D_{b}, being doses of the agents used on their own, are unchanged, so

$$\frac{{d}_{a}}{{D}_{\underline{a}}} + \frac{{d}_{b}}{{D}_{\underline{b}}} < 1 \qquad ( 2 )$$

When the opposite inequality, in which the combination of the dosage terms of Formula (2) is greater than 1, is fulfilled, then antagonism as defined by Berenbaum is present.

Therefore, to determine if the combined use of two drug regimens is synergistic compared to using the two drug regimens separately for the treatment, prevention, or slowing of the progression of AD or MCI, the following technique is used:

First, a patient or group of patients is treated with a GnRH-I analog for a period of time in a similar manner as shown in Example 4B. The dose level is doubled in treatment stages as above in Example 4B until no further cognitive or mental benefit to the patient or group of patients is obtained (in other words, a dose-response plateau is reached), or until the side effects from a higher dose of the GnRH-I analog outweigh the benefits to the patient or group of patients. At this point, it has been determined that an even higher dose of the GnRH-I analog will not be able to provide, or is unlikely to provide, an added benefit to a patient or a group of patients.

As a second step in this method of determining if synergy is present or not, the patient or group of patients is kept on the highest dose of the GnRH-I analog which has been determined to be of reasonable benefit to them, while a second drug-therapy regimen using an AChE inhibitor is begun. The clinician prescribes or administers a prudent initial dose of an AChEI drug, as suggested by the manufacturer or provider, to the patient or group of patients who are concomitantly on GnRH-I analog therapy. If, after a reasonable amount of time, such as a few days, a week, or two weeks, side effects due to the AChEI or combination are not present, or some side effects are present, but these do not outweigh the benefits to the patient or group of patients, then the next higher dose of AChEI, as recommended by a manufacturer or provider of the drug, is administered or prescribed for the patient or group of patients. This procedure is repeated until the increased dose of AChEI does not provide a significantly higher benefit to the patient or group of patients, or until it is determined that the side effects from the higher dose outweigh the cognitive benefits to the patient or patients.

As a third step to this method of synergy determination, the rate of cognitive decline or mental decline over a time period (such as a month, two months, three months, four months, five months, six months, seven months, eight months, nine months, ten months, eleven months or a year or more) of the patient or group of patients on the dual-therapy routine is scored using a technique known in the art, and this rate of decline is compared to the known rate of decline found for patients reported from clinical trials using the same or a similar monotherapy AChEI dose regimen as found useful for the AChEI part of the dual therapy, and using the same cognitive or other scoring method. Any cognitive or mental benefit, or decrease in side effects noticed for patients on the highest beneficial tolerated dose of AChEI in the dual therapy regimen, compared to the similar high-dose AChEI monotherapy reported for clinical trial results in the literature, such as lower rate of cognitive or mental decline with time in dual-therapy patients, over what is seen in the results for cognitive or mental decline with time in clinical trials using the same or similar AChEI monotherapy indicates that synergy is present in the combination therapy approach. The reason is as follows:

When, in a given instance, Berenbaum's inequality (2) is shown to be true, that is:

$$\frac{{d}_{a}}{{D}_{\underline{a}}} + \frac{{d}_{b}}{{D}_{\underline{b}}} < 1 \qquad ( 2 )$$

then synergy has been shown to be present. In the dual-therapy example above, it was determined that no further benefit, or very little significant benefit could be found using a higher dose of the GnRH-I analog in its corresponding monotherapy regimen, because either a dose-response plateau had been reached, or nearly reached, or side effects from the higher dose outweighed the benefits to patients. Further, it could be determined from the clinical literature that using a similar or nearly identical higher dose of the AChEI drug in a corresponding monotherapy regimen (i.e., from clinical trial published results) would not be of further benefit to patients, as side effects would outweigh the benefits to cognition at that dose.

Therefore, as explained by Berenbaum for this type of case where a maximum beneficial effect of one or the other agent is reached, in order for a more beneficial effect to be reached using either the GnRH-I analog or the AChEI drug alone, either Dₐ or D_{b} or both (i.e., the doses isoeffective with the combination effectiveness) used in Berenbaum's inequality (2) would have to be extremely large, and could be approximated to be infinite or to be near infinity. Therefore, when a maximum effect has been reached for one or both drug monotherapies, yet added benefit is seen from a combination of the therapies, either one or the other or both fractions in inequality (2) would be an extremely small number and the inequality showing synergy is thus shown to be true.

### Example 5B

### Synergy in Three-Drug Treatment Regimens, According to Embodiments of the Invention

In a similar manner to the method shown in Example 5A for dual-therapy, it is expected that a combination therapy involving administration of a GnRH-I analog, an AChE inhibitor, and an NMDA antagonist could be shown to be beneficial to patients, and that the outcome could be shown to be synergistic using Berenbaum's method extended to three drug participants. Again, in the cases where the maximum effects or near-maximal effects are known or can be determined for each individual component in method or composition, then synergy is determined to be present where a greater effect for the combination is found than for any component used separately, and where the sum of three ratios (instead of two ratios as shown in Equation (2)) adds up to a number less than 1.

### Example 6

### Clinical Trial Using a GnRH-I Analog for Reducing the Rate of Cognitive Decline with Time for Patients with AD, According to an Embodiment of the Invention

During 2004-2005, a 48-week, double-blind placebo-controlled dose ranging study was conducted in 108 women diagnosed with mild-to-moderate Alzheimer's Disease. The study inclusion criteria included a requirement that each patient either (a) is taking a cholinesterase inhibitor, began taking it at least 90 days prior to the trial and is likely to continue taking it at the same dosage level throughout the trial; or (b) has never taken a cholinesterase inhibitor or has stopped taking at least 90 days prior to the trial and is likely to remain off cholinesterase inhibitors throughout the trial. The patients in the subgroup taking cholinesterase inhibitors were in turn divided into two groups for analysis purposes:

Group 1 patients were administered an injectable 22.5 mg formulation of leuprolide acetate in combination with a stable dose of acetylcholinesterase inhibitors (AChEI). At the completion of the study, Group 1 included 24 subjects. Group 2 patients were administered a placebo injection (saline) in combination with a stable dose of AChEI. Group 2 included 26 subjects at the end of the study. Patients in a third study-treatment group (Group 3, N=12) were administered an injectable 22.5 mg formulation of leuprolide acetate at 12-week intervals over a 48-week period. The combined data from Group 3 are presented in Table 4. A placebo group was also used.

During the Phase II study, the administrations of leuprolide acetate and placebo occurred at weeks 0, 12, 24, 36, and 48 of the study, whereas doses of AChE inhibitors were taken as prescribed by a physician (determined as the proper beneficial level) to each specific patient before enrollment. As used in the study, a stable dose of AChEI meant that the patient took substantially the same formulation of AChEI, at substantially the same dosage amount and frequency, throughout the study period. The trial utilized the ADAS-Cog, an assessment of cognitive decline; the ADCS-ADL, an assessment of ability to perform activities of daily living; and the ADCS-CGIC, a clinician's assessment of the patient's cognitive state. These tests are commonly used assessments for primary endpoints in AD clinical trials.

Table 1 below shows the mean scores of the study participants on the ADAS-Cog test, which are also depicted in FIG. 1, along with the applicable statistical p-levels:

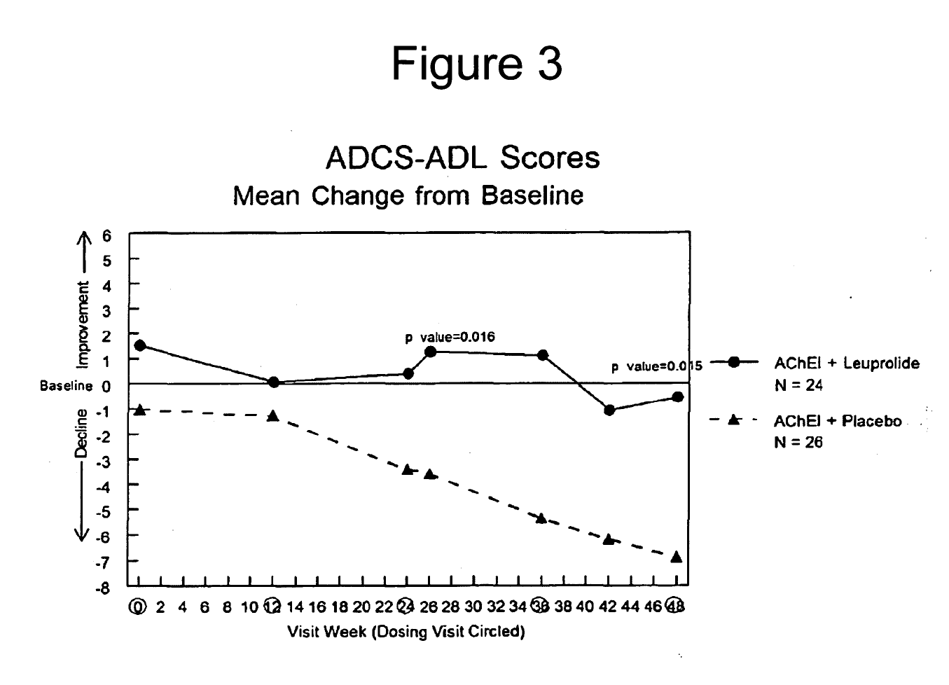

Table 2 below shows the mean scores of the study participants on the ADCS-ADL test, which are also depicted in FIG. 3, along with the applicable p-levels:

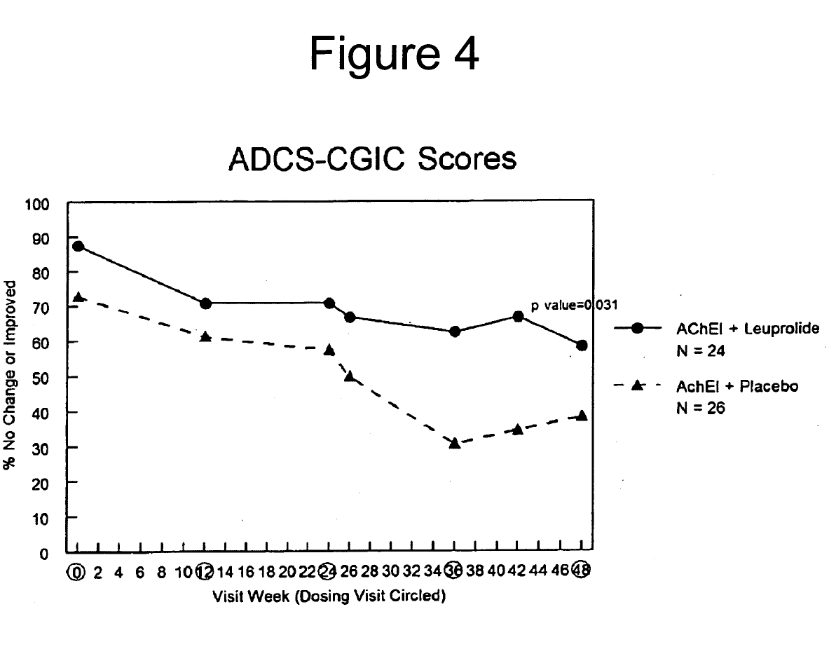

Table 3 reflects the scores of the study participants on the ADCS-CGIC test, which are also shown in FIG. 4, along with the applicable p-levels. Specifically, Table 3 and FIG. 4 show the proportion (percent) of patients in each group showing no change or improvement on the ADCS-CGIC test at various observation times during the trial.

TABLE 1

| | | Mean Change from Baseline | | | | | | |
|---|---|---|---|---|---|---|---|---|
| | Base- | Wk. | Wk. | Wk. | Wk. | Wk. | Wk. | Wk. |
| | line | 4 | 12 | 24 | 26 | 36 | 42 | 48 |
| Group 1 | 20.31 | −0.62 | 0.10 | 0.95 | −0.69 | 0.26 | 1.41 | 0.18 |
| Group 2 | 24.29 | 0.31 | 2.09 | 1.98 | 2.03 | 2.53 | 4.32 | 3.30 |

TABLE 2

| | Mean Change from Baseline | | | | | | |
|---|---|---|---|---|---|---|---|
| | Wk. | Wk. | Wk. | Wk. | Wk. | Wk. | Wk. |
| | 4 | 12 | 24 | 26 | 36 | 42 | 48 |
| Group 1 | 1.54 | 0.08 | 0.42 | 1.29 | 1.13 | −1.04 | −0.54 |
| Group 2 | −1.00 | −1.23 | −3.38 | −3.54 | −5.31 | −6.15 | −6.85 |

TABLE 3

| | Percent of Subjects Scoring No Change or Improvement | | | | | | |
|---|---|---|---|---|---|---|---|
| | Wk. | Wk. | Wk. | Wk. | Wk. | Wk. | Wk. |
| | 4 | 12 | 24 | 26 | 36 | 42 | 48 |
| Group 1 | 87.5 | 70.8 | 70.8 | 66.7 | 62.5 | 66.7 | 58.3 |
| Group 2 | 73.0 | 61.5 | 57.7 | 50.0 | 30.8 | 34.6 | 38.5 |

As shown in FIG. 2, females receiving AChEIs only declined 3.3 points over 48 weeks on the ADAS-cog outcome measure. Females receiving 11.25 mg Lupron Depot® alone declined 16 points over 48 weeks, whereas females receiving 22.5 mg Lupron Depot® declined 4.5 points over 48 weeks. When females were administered 11.25 mg Lupron Depot® together with AChEIs, they declined approximately 4 points while those receiving 22.5 mg Lupron Depot® and AChEIs did not worsen, but instead were stable at the end of 48 weeks.

An analysis of these data indicates, at statistically significant levels, that the mean ADAS-Cog scores for Group 1 (combination of AChEI and 22.5 mg dosage of leuprolide acetate) remained essentially at baseline (a decline of 0.18 points) compared to a decline of 3.3 points in the AChEI plus placebo group (Group 2), with an unadjusted p-value of 0.026. The mean ADCS-ADL score in Group 1 (GnRH-I analog+AChEI) also remained essentially at baseline (a decline of 0.54 points) compared to a decline in Group 2 (AChEI plus placebo) of 6.85 points, with an unadjusted p-value of 0.015. In the ADCS-CGIC tests, 58% of the patients in Group 1 scored “no change” or “improvement” at week 48, versus 38% of the patients in Group 2.

Table 4 shows the results on the ADAS-cog (mean change from baseline), ADCS-ADL (mean change from baseline) and ADAS-CGIC tests (percent no change or improvement) for a group of patients (N=12) administered an injectable 22.5 mg formulation of leuprolide acetate at 12-week intervals over a 48-week period, but taking no other cognition-related treatment drug.

TABLE 4

| | Base- | Wk. | Wk. | Wk. | Wk. | Wk. | Wk. | Wk. |
|---|---|---|---|---|---|---|---|---|
| | line | 4 | 12 | 24 | 26 | 36 | 42 | 48 |
| ADAS- | 19.79 | 2.17 | 2.99 | 3.94 | 1.20 | 3.24 | 5.22 | 4.68 |
| cog | | | | | | | | |
| ADCS- | | −2.75 | −1.92 | −4.83 | −4.58 | −5.17 | −5.17 | −6.50 |
| ADL | | | | | | | | |
| ADCS- | | 66.7% | 50% | 41.7% | 41.7% | 50% | 50% | 25% |
| CGIC | | | | | | | | |

These expected cognitive declines are similar in mild-to-moderate Alzheimer's patients who are untreated, suggesting that AChEIs do not provide sustained benefit. In contrast, non-demented elderly subjects do not suffer cognitive decline as a normal part of aging.

The average rates of decline in these populations can be used to judge the efficacy of the combination therapy treatments. These data are known for large groups of people, and allow for a very reliable rate of decline for each group. In this study, the placebo group of patients was very small, and did not follow the normal rate of decline over the trial study time which would be expected from comparisons with data found in published materials.

Analysis of these data also suggests that the combination of leuprolide acetate with acetylcholinesterase inhibitors has a greater effect on preventing or slowing the progress of AD than the additive effects of the two drugs administered alone.

The clinical trial also involved AD patients who were using NMDA receptor antagonists concomitantly with leuprolide acetate. Anecdotal evidence from the trial also suggests that the use of a combination of leuprolide acetate and NMDA receptor antagonists also has a greater effect on preventing or slowing the progress of AD than the additive effects of the two drugs administered separately.

### Example 7

### Pharmacokinetics of GnRH-I Analog Depot Formulations

The clinical data presented in this specification in Example 6 was from a clinical trial of females treated with Lupron Depot®, an injectable formulation of leuprolide acetate with a well-defined pharmacokinetic drug release profile (Lupron Depot® Package Insert available at http://www.Lupron Depot.com/prostate/PackageInsert.asp) that is characterized by a large percentage of the available drug being released during the initial 72 hours after injection. This is very effective at lowering gonadotropin production by the pituitary to a level sufficient to decrease sex steroid production by the testes or ovaries, thereby providing benefit to men with prostate cancer or women with endometriosis, respectively. For Alzheimer's disease therapy, in some cases, it is possible that a “burst” release of drug during the initial post-dosing period is important. Therefore, a formulation of GnRH-I analog used to treat Alzheimer's disease or MCI is prepared with this important “burst” release profile in mind to have similar pharmacokinetic characteristics to those seen with Lupron Depot®. Such a formulation is prepared using a similar formulation of polymer containing an analog such as leuprolide, nafarelin, buserelin, histrelin, triptorelin, goserelin, cetrorelix, abarelix, ganirelix, or other GnRH-I analog. For some formulations of GnRH-I analog, it is found that an injectable formulation with the above release profile in combination with a GnRH-I analog implant with a steadier, more uniform release of the same or different GnRH-I analog is preferable as a therapeutic combination for patient treatment; i.e., causing an initial down-regulation of receptor with the burst release profile followed by the sustained effect due to the presence of higher tissue or serum levels, due to the steadier drug delivery by an implant.

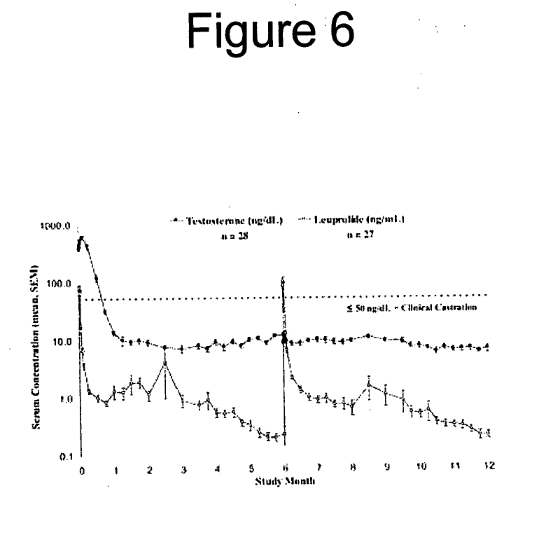

FIG. 6 shows FIG. 6 shows a pharmacokinetic release profile of leuprolide acetate and a decrease in serum concentration of testosterone in men receiving ELIGARD® 45 mg. As shown in FIG. 6, the pharmacokinetic release profile of ELIGARD® 45 mg in 27 men with advanced prostate cancer demonstrates the initial “burst” release of leuprolide acetate during the first few days after dosing (leuprolide acetate serum concentration reaches approximately 100 ng/ml). At a six month re-dosing interval, the “burst” leuprolide release is again demonstrated.

Injectable formulations of GnRH-I analogs have similar release profiles characterized by a “burst” of drug release during the first few days followed by declining levels for the remainder of the dose. Lupron Depot®, TRELSTAR® Depot, Supprellin, Cetrotide® and Decapeptyl® SR are all characterized by this release profile.

### Exemplary Formulations

As mentioned above, GnRH-I agonists are small peptides, and as such are generally not amenable to oral administration. Therefore, they are customarily administered subcutaneously, intramuscularly, or via nasal spray. In some embodiments, the GnRH-I analog, e.g., leuprolide, goserelin, nafarelin, buserelin, histrelin, triptorelin, cetrorelix, abarelix, or ganirelix is provided for administration in a formulation, comprising a free base form, or a salt form, in which the method for administration of pharmaceutically-active material comprises the drug administration to the patient at a rate comprising twice a day, daily, weekly, monthly, quarterly, or yearly or in such combinations of units of time as deemed necessary to provide the desired cognition effects in addition to being useful or convenient to the patient or care-giver. Many patients and care-givers find that monthly injections of GnRH-I analog, or injections of analog administered at 2-month, 3-month, 4-month, 5-month, 6-month, or yearly intervals are more convenient and better tolerated than injections of GnRH-I analog administered more frequently. Other embodiments for administration methods comprise using a slow-release formulation as found in products such as: Lupron Depot®Viadur™, ELIGARD®, Zoladex®, Synarel®, TRELSTAR® DEPOT, SUPPRELIN® LA, VANTAS®, SUPREFACT®, and Decapeptyl® SR for the treatment of AD or MCI, as described further in Tables A-G and Examples 1-7.

Another more specific example of a slow-release formulation of a GnRH-I analog which can be used in the method of the invention is that obtained from Durect Corporation of Cupertino, Calif. under the trade name DURIN. This formulation is a solid formulation comprising approximately 25-30 weight % leuprolide acetate dispensed in a matrix of poly (DL-lactide-co-glycolide). The formulation is a cylindrical, opaque rod with nominal dimensions of approximately 1.5 mm (diameter) by approximately 2.0 cm (length). This formulation can be implanted into a patient about every one month, two months, or three months to provide approximately 11.25 mg leuprolide per 2 cm rod, and to provide a substantially uniform release profile over the time period it is left in. Preferably, the time period before replacement or addition of one or more other implants is two months. More than one piece of DURIN formulation or similar material can be implanted; for example, two 1.5 mm×2 cm implants can be inserted via trochar or other device or method suitable for surgical placement of the formulation in a patient. In some patients, especially those who are overweight or are very large, the introduction of 3 or even 4 or more pieces of DURIN formulation or of a similar material containing a different GnRH-I analog can be used in the method of the invention. GnRH-I analogs such as leuprolide have been shown to be metabolized by peptidases, and the cytochrome P450 enzymes are not known to be involved.

Acetylcholinesterase inhibitors and NMDA receptor antagonists are orally available and generally delivered in tablet or liquid form. Donepezil is metabolized by cytochrome P450 enzymes into multiple metabolites. Rivastigmine is metabolized through the action of hydrolysis by esterases. Galantamine is metabolized by hepatic cytochrome P450 enzymes. Tacrine is metabolized by cytochrome P450 enzymes into multiple metabolites. Memantine undergoes little metabolism, with the majority (up to 82%) of a dose being excreted in the urine unchanged; the remainder is converted to three polar metabolites.

Given the different availabilities and routes of metabolism, it is expected that two or more of GnRH-I agonists, AChE inhibitors, or NMDA receptor antagonists will be administered in a combination therapy that may or may not be in a single dosage form.

While various embodiments of the present invention have been described above, it should be understood that they have been presented by way of example only, and not by way of limitation. The breadth and scope of the present invention should not be limited to any of the above-described exemplary embodiments, but should be defined in accordance with the appended claims.

1. A method of treating, mitigating, slowing the progression of, or preventing a condition selected from the group consisting of Alzheimer's disease and Mild Cognitive Impairment (MCI), comprising the step of:
administering a therapeutically effective combination of a gonadotropin-releasing hormone analog with an acetylcholinesterase inhibitor or an N-methyl-D-aspartate receptor antagonist.

2-3. (canceled)

4. A method of treating, mitigating, slowing the progression of, or preventing Alzheimer's disease, comprising the step of:
administering a therapeutically effective synergistic combination of a gonadotropin-releasing hormone analog with an acetylcholinesterase inhibitor or an N-methyl-D-aspartate receptor antagonist.

5. The method of claim 1, wherein the gonadotropin releasing hormone is leuprolide acetate, and the acetylcholinesterase inhibitor is a selected from the group consisting of donepezil, rivastigimine, galantamine and tacrine.

6. (canceled)

7. The method of claim 1, wherein the gonadotropin releasing hormone is leuprolide acetate, and the N-methyl-D-aspartate receptor antagonist is memantine.

8. The method of claim 1, wherein the gonadotropin releasing hormone is leuprolide acetate, and the N-methyl-D-aspartate receptor antagonist is memantine.

9. The method of claim 4, wherein the therapeutically effective synergistic combination comprises a therapeutically effective amount of an acetylcholinestarase inhibitor selected from the group consisting of donepezil, rivastigimine, galantamine and tacrine and a therapeutically effective amount of an N-methyl-D-aspartate receptor antagonist.

10. The method of claim 9, wherein the N-methyl-D-aspartate receptor antagonist is memantine.

11. The method of claim 4, wherein the therapeutically effective synergistic combination is a therapeutically effective synergistic combination of leuprolide acetate and an acetylcholinestarase inhibitor selected from the group consisting of donepezil, rivastigimine, galantamine and tacrine.

12. The method of claim 4, wherein the therapeutically effective synergistic combination is a therapeutically effective synergistic combination of leuprolide acetate and memantine.

13. The method of claim 1, wherein the gonadotropin-releasing hormone analog comprises leuprolide and is administered approximately once every 60 days in combination with a stable dose of an acetylcholinesterase inhibitor.

14. The method of claim 1, wherein the combination comprises approximately 22.5 mg of leuprolide acetate.

15. The method of claim 14, wherein the leuprolide acetate is administered in a controlled-release formulation.

16. A combination comprising:
a gonadotropin-releasing hormone analog and at least one of an acetylcholinesterase inhibitor and an N-methyl-D-aspartate receptor antagonist.

17. The combination of claim 16, wherein the gonadotropin-releasing hormone comprises leuprolide acetate and the acetylcholinesterase inhibitor is selected from the group consisting of donepezil, rivastigimine, galantamine and tacrine.

18. The combination of claim 16, wherein the gonadotropin-releasing hormone comprises leuprolide acetate and the N-methyl-D-aspartate receptor antagonist comprises memantine.

19. The combination of claim 16, wherein the gonadotropin-releasing hormone analog has the following structural formula: 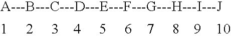
or a pharmaceutically acceptable salt thereof; wherein:
A is an amino acyl residue selected from the group consisting of:
L-pyroglutamyl, D-pyroglutamyl, N-acetyl-L-prolyl, N-acetyl-D-prolyl, N-acetyl-L-delta^{3,4}-prolyl, N-acetyl-D-delta^{3,4}-prolyl, N-acetyl-L phenylalanyl, N-acetyl-D-phenylalanyl, N-acetyl-L-3-(2-thienyl)alanyl, N-acetyl-D-3-(2-thienyl)alanyl, N-acetyl-L-3-(4-chlorophenyl)alanyl, N-acetyl-D-3-(4-chlorophenyl)alanyl, N-acetyl-L-3-(4-fluorophenyl)alanyl, N-acetyl-D-3-(4-fluorophenyl)alanyl, N-acetyl-L-3-(4-bromophenyl)alanyl, N-acetyl-D-3-(4-bromophenyl)alanyl, N-acetyl-L-3-(4-methylphenyl)alanyl, N-acetyl-D-3-(4-methylphenyl)alanyl, N-acetyl-L-3-(pentamethylphenyl)alanyl, N-acetyl-D-3-(pentamethylphenyl)alanyl, N-acetyl-L-3-(3,4,5-trimethylphenyl)alanyl, N-acetyl-D-3-(3,4,5-trimethylphenyl)alanyl, N-acetyl-L-3-tryptyl(N-indole-methyl), N-acetyl-D-3-tryptyl(N-indole-methyl), N-acetyl-L-tryptyl(N-indole-formyl), N-acetyl-D-tryptyl-(N-indole-formyl), N-acetyl-L-3-(1-adamantyl)alanyl, N-acetyl-D-3-(1-adamantyl)alanyl, N-acetyl-L-5-fluorotryptyl(N-indole-formyl), N-acetyl-D-5-fluorotryptyl(N-indole-formyl), N-acetyl-L-3-(2-naphthyl)alanyl, N-acetyl-D-3-(2-naphthyl)alanyl, N-acetyl-L-3-(3-benzothienyl)alanyl, N-acetyl-D-3-(3-benzothienyl)alanyl, N-acetyl-L-3-(3-benzoxazolyl)alanyl, N-acetyl-D-3-(3-benzoxazolyl)alanyl, N-acetyl-alpha-methyl-L-3-(4-chlorophenyl)alanyl, N-acetyl-alpha-methyl-D-3-(4-chlorophenyl)alanyl, N-acetyl-L-3-(4-trifluoromethylphenyl)alanyl, N-acetyl-D-3-(4-trifluoromethylphenyl)alanyl, N-acetyl-L-tyrosyl, N-acetyl-D-tyrosyl, N-acetyl-L-O-methyl-tyrosyl, N-acetyl-D-O-methyl-tyrosyl, N-acetyl-D-3-(2-naphthyl)alanyl, N-acetyl-L-3-(1-naphthyl)alanyl, N-acetyl-D-3-(1-naphthyl)alanyl, N-acetylsarcosyl, N-acetyl-L-3-(cyclohexyl)alanyl, N-acetyl-D-3-(cyclohexyl)alanyl, N-acetylglycyl, L-N-acetyl-N-methylalanyl, N-acetyl-N-methyl-D-alanyl, N-acetyl-alpha-methyl-L-phenylalanyl, N-acetyl-alpha-methyl-D-phenylalanyl, N-acetyl-D-phenylalanyl, N-acetyl-L-phenylalanyl, N-formylsarcosyl, N-formyl-N-methyl-L-alanyl, N-formyl-N-methylalanyl, 2-N-beta-(ethylaminocarbonyl)-N-epsilon-(ethylamido)glutamyl, N-delta-ethyl-glutamyl, L-prolyl, D-prolyl, L-delta^{3,4}-prolyl, D-delta^{3,4}-prolyl, L-phenylalanyl, D-phenylalanyl, L-3-(4-methylphenyl)alanyl), D-3-(4-methylphenyl)alanyl, L-3-(4-nitrophenyl)alanyl, D-3-(4-nitrophenyl)alanyl, L-3-(4-acetylaminophenyl)alanyl, D-3-(4-acetylaminophenyl)alanyl, L-3-(4-chlorophenyl)alanyl, D-3-(4-chlorophenyl)alanyl, L-3-(4-fluorophenyl)alanyl, D-3-(4-fluorophenyl)alanyl, alpha-methyl-L-3-(4-chlorophenyl)alanyl, alpha-methyl-D-3-(4-chlorophenyl)alanyl, L-3-(4-trifluoromethylphenyl)alanyl, D-3-(4-trifluoromethylphenyl)alanyl, L-tyrosyl, D-tyrosyl, L-O-methyl-tyrosyl, D-O-methyl-tyrosyl, sarcosyl, glycyl, L-N-methylalanyl, N-methyl-D-alanyl, N-methyl-L-pyroglutamyl, N-methyl-D-pyroglutamyl, alpha-methyl-L-phenylalanyl, alpha-methyl-D-phenylalanyl, N-acetyl-alpha-aza-3-(4-chlorophenyl)alanyl, N-acetyl-alpha-aza-3-(4-fluorophenyl)alanyl, N-acetyl-alpha-aza-3-(2-naphthyl)alanyl, N-acetyl-alpha-aza-3-(1-naphthyl)alanyl, N-acetyl-alpha-aza-alanyl, N-acetyl-alpha-aza-glycyl, N-acetyl-alpha-aza-sarcosyl, N-acetyl-alpha-aza-3-(4-methylphenyl)alanyl, N-acetyl-alpha-aza-cyclohexylalanyl, N-acetyl-alpha-aza-3-(1-adamantyl)alanyl, N-acetyl-alpha-aza-tyrosyl(O-methyl), N-acetyl-alpha-aza-3-(3-benzothienyl)alanyl, N-acetyl-alpha-aza-phenylalanyl, N-methyl-alpha-aza-pyroglutamyl, N-acetyl-alpha-aza-3-(2-thienyl)alanyl, N-acetyl-alpha-aza-3-(3-benzoxazolyl)alanyl, N-acetyl-alpha-aza-3-(3,4,5-trimethylphenyl)alanyl, N-acetyl-alpha-aza-3-(pentamethylphenyl)alanyl, N-acetyl-N-alpha-methyl-alpha-aza-3-(2-naphthyl)alanyl, N-acetyl-N-alpha-methyl-alpha-aza-3-(1-naphthyl)alanyl, N-acetyl-N-alpha-methyl-alpha-aza-3-(4-chlorophenyl)alanyl, N-acetyl-N-alpha-methyl-alpha-aza-3-(4-fluorophenyl)alanyl, N-acetyl-N-alpha-methyl-alpha-aza-3-(4-methylphenyl)alanyl, N-acetyl-N-alpha-methyl-alpha-aza-3-(4-methoxyphenyl)alanyl, N-acetyl-N-alpha-methyl-alpha-aza (1-adamantyl)alanyl, N-acetyl-N-alpha-methyl alpha-aza-3-(phenyl)alanyl, N-acetyl-N-alpha-methyl-alpha-aza-alanyl, N-acetyl-N-alpha-methyl alpha-aza-3-(cyclohexyl)alanyl, N-acetyl-N-alpha-methyl-alpha-aza-3-(benzothienyl)alanyl, N-acetyl-N-alpha-methyl-alpha-aza-3-(benzoxazolyl)alanyl, N-acetyl-N-alpha-methyl-alpha-aza-3-(3,4,5-trimethylphenyl)alanyl, N-acetyl-N-alpha-methyl-alpha-aza-3-(pentamethylphenyl)alanyl and N-acetyl-N-alpha-methyl-alpha-aza-3-(2-thienyl)alanyl;
B is absent or is an amino acyl residue selected from the group consisting of L-histidyl, D-histidyl, L-tryptyl, D-tryptyl, L-tryptyl(N-indole-methyl), D-tryptyl(N-indole-methyl), L-phenylalanyl, D-phenylalanyl, L-3-(2-naphthyl)-alanyl, D-3-(2-naphthyl)-alanyl, L-3-(1-naphthyl)-alanyl, D-3-(1-naphthyl)-alanyl, L-3-(3-benzoxazolyl)alanyl, D-3-(3-benzoxazolyl)alanyl, L-3-(3-pyridyl)-alanyl, L-3-(2-pyridyl)-alanyl, D-3-(3-pyridyl)-alanyl, D-3-(2-pyridyl)-alanyl, L-3-(2-thiazolyl)alanyl, D-3-(2-thiazolyl)-alanyl, L-3-(3-benzothienyl)alanyl, D-3-(3-benzothienyl)alanyl, L-3-(2-benzothienyl)alanyl, D-3-(2-benzothienyl)alanyl, L-3-(2-thienyl)-alanyl, D-3-(2-thienyl)-alanyl, L-cyclohexylalanyl, D-cyclohexylalanyl, L-3-(3-pyrazolyl)alanyl, D-3-(3-pyrazolyl)alanyl, L-3-(4-chlorophenyl)alanyl, D-3-(4-chlorophenyl)alanyl, L-3-(4-fluorophenyl)alanyl, D-3-(4-fluorophenyl)alanyl, L-3-(4-bromophenyl)alanyl, D-3-(4-bromophenyl)alanyl, L-3-(4-trifluoromethylphenyl)alanyl, D-3-(4-trifluoromethylphenyl)alanyl, L-3-(4-aminophenyl)alanyl, D-3-(4-aminophenyl)alanyl, L-3-(4-nitrophenyl)alanyl, D-3-(4-nitrophenyl)alanyl, L-3-(4-cyanophenyl)alanyl, D-3-(4-cyanophenyl)alanyl, L-tyrosyl-(O-methyl), D-tyrosyl(O-methyl), L-3-(4-methylphenyl)alanyl, D-3-(4-methylphenyl)alanyl, L-3-(4-nitrophenyl)alanyl, D-3-(4-nitrophenyl)alanyl, L-3-(4-acetylaminophenyl)alanyl, D-3-(4-acetylaminophenyl)alanyl, L-methionyl, D-methionyl, L-alpha-methyl-3-(4-chlorophenyl)alanyl, D-alpha-methyl-3-(4-chlorophenyl)alanyl, (3S)-1,2,3,4-tetrahydroisoquinoline-3-carbonyl, (3R)-1,2,3,4-tetrahydroisoquinoline-3-carbonyl, (2)-N-(ethylaminocarbonyl)-(5)-N-(ethylamido)glutamyl, alpha-aza-3-(3,4,5-trimethylphenyl)alanyl, alpha-aza-3-(4-bromophenyl)alanyl, alpha-aza-3-(4-methylphenyl)alanyl, alpha-aza-3-(1-naphthyl)alanyl, alpha-aza-3-(1-adamantyl)alanyl, L-3-(3-quinolyl)-alanyl, D-3-(3-quinolyl)-alanyl, alpha-aza-3-(4-chlorophenyl)alanyl, alpha-aza-3-(4-fluorophenyl)alanyl, alpha-aza-3-(2-naphthyl)alanyl, alpha-aza-3-(3-quinolyl)alanyl, alpha-aza-phenylalanyl, alpha-aza-tyrosyl(O-methyl), alpha-aza-3-(2-thienyl)alanyl, alpha-aza-3-(3-benzthienyl)alanyl, alpha-aza-cyclohexylalanyl, alpha-aza-tryptyl, alpha-aza-tryptyl(N-indole-methyl), alpha-aza-tryptyl(N-indole-formyl), N—(R₃₁)-L-phenylalanyl, N—(R₃₁)-D-phenylalanyl, N—(R₃₁)-D-3-(4-chlorophenyl)alanyl, N—(R₃₁)-L-3-(4-chlorophenyl)alanyl, N—(R₃₁)-D-3-(4-fluorophenyl)alanyl, N—(R₃₁)-L-3-(4-fluorophenyl)alanyl, N—(R₃₁)-L-3-(4-trifluoromethylphenyl)alanyl, N—(R₃₁)-D-3-(4-trifluoromethylphenyl)alanyl, N—(R₃₁)-L-3-(cyclohexyl)alanyl, N—(R₃₁)-D-3-(cyclohexyl)alanyl, N—(R₃₁)-L-3-(4-bromophenyl)alanyl, N—(R₃₁)-D-3-(4-bromophenyl)alanyl, N—(R₃₁)-L-3-(4-nitrophenyl)alanyl, N—(R₃₁)-D-3-(4-nitrophenyl)alanyl, L-prolyl, D-prolyl, N—(R₃₁)-L-O-methyltyrosyl, N—(R₃₁)-L-tyrosyl, N—(R₃₁)-D-O-methyl-tyrosyl, N—(R₃₁)-D-tyrosyl, N—(R₃)-L-histidyl, N—(R₃₁)-D-histidyl, N—(R₃₁)-L-3-(2-thienyl)alanyl, N—(R₃₁)-D-3-(2-thienyl)alanyl, N—(R₃₁)-L-3-(2-thiazolyl)alanyl, N—(R₃₁)-D-3-(2-thiazolyl)alanyl, N—(R₃₁)-L-3-(2-pyridyl)alanyl, N—(R₃₁)-D-3-(2-pyridyl)alanyl, N—(R₃₁)-D-3-(2-naphthyl)alanyl, N—(R₃₁)-L-3-(2-naphthyl)alanyl, N—(R₃₁)-L-3-(3-benzothienyl)alanyl, N—(R₃₁)-D-3-(3-benzothienyl)alanyl, N—(R₃₁)-L-3-(2-benzothienyl)alanyl, N—(R₃₁)-D-3-(2-benzothienyl)alanyl, N—(R₃₁)-L-3-(3-benzoxazolyl)alanyl, N—(R₃₁)-D-3-(3-benzoxazolyl)alanyl, N—(R₃₁)-L-3-(3-pyridyl)alanyl, N—(R₃₁)-D-3-(3-pyridyl)alanyl, N—(R₃₁)-L-tryptyl, N—(R₃₁)-D-tryptyl, N—(R₃₁)-L-tryptyl(N-indole-methyl), N—(R₃₁)-D-tryptyl(N-indole-methyl), N—(R₃₁)-D-methionyl, N—(R₃₁)-L-methionyl, N—(R₃₁)-D-3-(1-naphthyl)alanyl, and N—(R₃₁)-L-3-(1-naphthyl)alanyl, wherein R₃₁— is methyl, ethyl, 2-fluoroethyl, 2,2-difluoroethyl, 2,2,2-trifluoroethyl, propyl or isopropyl;
C is an amino acyl residue selected from the group consisting of L-tryptyl, D-tryptyl, L-tryptyl(N-indole-formyl), D-tryptyl(N-indole-formyl), L-tryptyl(N-indole-methyl), D-tryptyl(N-indole-methyl), 5-fluoro-L-tryptyl, 5-fluoro-D-tryptyl, L-phenylalanyl, L-prolyl, D-prolyl, L-tyrosyl, D-tyrosyl, D-phenylalanyl, D-3-(3-pyridyl)alanyl, L-3-(3-pyridyl)alanyl, D-3-(3-pyridyl-N′-oxide)alanyl, L-3-(3-pyridyl-N′-oxide)alanyl, D-3-(3-quinolyl)alanyl, L-3-(3-quinolyl)alanyl, D-3-(3-quinolyl-N′-oxide)alanyl, L-3-(3-quinolyl-N′-oxide)alanyl, D-3-(1-adamantyl)alanyl, L-3-(1-adamantyl)alanyl, L-3-(1-naphthyl)alanyl, D-3-(1-naphthyl)alanyl, L-3-(3-benzothienyl)alanyl, D-3-(3-benzothienyl)alanyl, L-3-(2-benzothienyl)alanyl, D-3-(2-benzothienyl)alanyl, L-3-(3-benzoxazolyl)alanyl, D-3-(3-benzoxazolyl)alanyl, L-cyclohexylalanyl, D-cyclohexylalanyl, L-3-(3-indazolyl)alanyl, D-3-(3-indazolyl)alanyl, alpha-methyl-L-phenylalanyl, alpha-methyl-D-phenylalanyl, L-3-(2-naphthyl)alanyl, D-3-(2-naphthyl)alanyl, L-O-methyltyrosyl, D-O-methyltyrosyl, L-3-(4-methylphenyl)alanyl, D-3-(4-methylphenyl)alanyl, L-3-(pentamethylphenyl)alanyl, D-3-(pentamethylphenyl)alanyl, L-3-(3,4,5-trimethylphenyl)alanyl, D-3-(3,4,5-trimethylphenyl)alanyl, L-3-(4-chlorophenyl)alanyl, D-3-(4-chlorophenyl)alanyl, alpha-methyl-L-3-(4-chlorophenyl)alanyl, alpha-methyl-D-3-(4-chlorophenyl)alanyl, L-3-(4-trifluoromethylphenyl)alanyl, D-3-(4-trifluoromethylphenyl)alanyl, L-3-(4-fluorophenyl)alanyl, D-3-(4-fluorophenyl)alanyl, L-3-(2-thienyl)-alanyl, D-3-(2-thienyl)-alanyl, N—(R₃₂)-L-3-(3-pyridyl)alanyl, N—(R₃₂)-D-3-(3-pyridyl)alanyl, N—(R₃₂)-L-3-(3-pyridyl-N′-oxide)alanyl, N—(R₃₂)-D-3-(3-pyridyl-N′-oxide)alanyl, L-3-(2-thiazolyl)-alanyl, D-3-(2-thiazolyl)alanyl, alpha-aza-3-(1-naphthyl)alanyl, alpha-aza-tryptyl, alpha-aza-phenylalanyl, alpha-aza-3-(2-thienyl)alanyl, alpha-aza-3-4-methylphenyl)alanyl, alpha-aza-3-(pentamethylphenyl)alanyl, alpha-aza-3-(2-naphthyl)alanyl, alpha-aza-3-(3-benzothienyl)alanyl, alpha-aza-3-(3-benzoxazolyl)alanyl, alpha-aza-3-(cyclohexyl)alanyl, alpha-aza-3-(1-adamantyl)alanyl, alpha-aza-3-(4-methoxyphenyl)alanyl, alpha-aza-3-(4-chlorophenyl)alanyl, alpha-aza-3-(4 bromophenyl)alanyl, alpha-aza-tryptyl(N-indole-methyl), alpha-aza-3-(3-pyridyl)alanyl, alpha-aza-3-(3-quinolyl)alanyl, alpha-aza-3-(2-thiazolyl)alanyl, N—(R₃₂)-L-3-(2-thienyl)alanyl, N—(R₃₂)-D-3-(2-thienyl)alanyl, L-3-(3-quinolyl)alanyl, D-3-(3-quinolyl)alanyl, L-3-(2-naphthyl)alanyl, D-3-(2-naphthyl)alanyl, N—(R₃₂)-D-phenylalanyl, N—(R₃₂)-L-phenylalanyl, N—(R₃₂)-D-tryptyl, N—(R₃₂)-L-tryptyl, N—(R₃₂)-L-tryptyl(N-indole-formyl), N—(R₃₂)-D-tryptyl(N-indole-formyl), N—(R₃₂)-L-tryptyl(N-indole-methyl), N—(R₃₂)-D-tryptyl(N-indole-methyl), N-(R₃₂)-L-3-(2-thiazolyl)alanyl, N—(R₃₂)-D-3-(2-thiazolyl)alanyl, N—(R₃₂)-L-3-(3-pyridyl)alanyl, N—(R₃₂)-D-3-(3-pyridyl)alanyl, N—(R₃₂)-D-3-(3-quinolyl)alanyl, N—(R₃₂)-L-3-(3-quinolyl)alanyl, N—(R₃₂)-D-3-(1-adamantyl)alanyl, N—(R₃₂)-L-3-(1-adamantyl)alanyl, N—(R₃₂)—)-D-3-(4-fluorophenyl)alanyl, N—(R₃₂)-L-3-(4-fluorophenyl)alanyl, N—(R₃₂)-D-3-(4-chlorophenyl)alanyl, N—(R₃₂)-L-3-(4-chlorophenyl)alanyl, N—(R₃₂)-L-3-(4-trifluoromethylphenyl)alanyl, N—(R₃₂)-D-3-(4-trifluoromethylphenyl)alanyl, N—(R₃₂)-D-3-(2-naphthyl)alanyl, N—(R₃₂)-L-3-(2-naphthyl)alanyl, N—(R₃₂)-D-3-(1-naphthyl)alanyl, N—(R₃₂)-L-3-(1-naphthyl)alanyl, N—(R₃₂)-L-3-(3-benzothienyl)alanyl, N—(R₃₂)-D-3-(3-benzothienyl)alanyl, N—(R₃₂)-L-3-(2-benzothienyl)alanyl, N—(R₃₂)-D-3-(2-benzothienyl)alanyl, N—(R₃₂)-L-3-(3-benzoxazolyl)alanyl, N—(R₃₂)-D-3-(3-benzoxazolyl)alanyl, N—(R₃₂)-L-tyrosyl, N—(R₃₂)-D-tyrosyl, N—(R₃₂)-L-3-(3,4,5-trimethylphenyl)alanyl, N—(R₃₂)-D-3-(3,4,5-trimethylphenyl)alanyl, N—(R₃₂)-L-3-(4-methylphenyl)alanyl, N—(R₃₂)-D-3-(4-methylphenyl)alanyl, N—(R₃₂)-L-3-(pentamethylphenyl)alanyl, N—(R₃₂)-D-3-(pentamethylphenyl)alanyl, N—(R₃₂)-L-3-(4-bromophenyl)alanyl, N—(R₃₂)-D-3-(4-bromophenyl)alanyl, N—(R₃₂)-L-cyclohexylalanyl, N—(R₃₂)-D-cyclohexylalanyl, N—(R₃₂)-L-3-(3-indazolyl)alanyl, N—(R₃₂)-D-3-(3-indazolyl)alanyl, N-alpha-N—(R₃₂)-alpha-aza-3-(1-naphthyl)alanyl, N-alpha-(R₃₂)-alpha-aza-3-(3-pyridyl)alanyl, N-alpha-(R₃₂)-alpha-aza-phenylalanyl, N-alpha-(R₃₂)-alpha-aza-3-(3-benzothienyl)alanyl, N-alpha-(R₃₂)-alpha-aza-3-(2-benzothienyl)alanyl, N-alpha-(R₃₂)-alpha-aza-3-(4-methylphenyl)alanyl, N-alpha-(R₃₂)-alpha-aza-3-(4-methylphenyl)alanyl, N-alpha-(R₃₂)-alpha-aza-3-(4-chlorophenyl)alanyl, N—(R₃₂)—O-methyl-D-tyrosyl and N—(R₃₂)—O-methyl-L-tyrosyl, wherein R₃₂ is methyl, ethyl, 2-fluoroethyl, 2,2-difluoroethyl, 2,2,2-trifluoroethyl, propyl or isopropyl;
D is an amino acyl residue selected from the group consisting of prolyl, 4-hydroxyproline, L-seryl, L-seryl(O-benzyl), L-seryl(O—PO₃H₂), L-seryl(O—PO₃Me₂), D-seryl, D-seryl(O-benzyl), D-seryl(O—PO₃H₂), D-seryl(O—PO₃Me₂), L-glutamine, D-3-(3-pyridyl)alanyl, L-3-(3-pyridyl)alanyl, D-3-(3-pyridyl-N′-oxide)alanyl, L-3-(3-pyridyl-N′-oxide)alanyl, D-3-(3-quinolyl)alanyl, L-3-(3-quinolyl)alanyl, D-3-(3-quinolyl-N′-oxide)alanyl, L-3-(3-quinolyl-N′-oxide)alanyl, L-alpha,beta-diaminopropionyl, L-alanyl, L-threonyl, 2,3-diaminopropionyl, 2-amino-3-guanidinopropionyl, 2,3-diaminopropionyl (wherein the 3-amino group is substituted with lower alkyl, 3-pyridinecarbonyl, 2-pyrazinecarbonyl or 2-indolecarbonyl), N-alpha-aza-glycyl, N-alpha-aza-alanyl, N-alpha-(R₀)-alpha-aza-glycyl, N-alpha-(R₀)-alpha-aza-alanyl, N-alpha-(R₀)-L-seryl, N-alpha-(R₀)-L-seryl(O-benzyl), N N-alpha-(R₀)-L-glutamine, N-alpha-(R₀)-L-alanyl, N-alpha-(R₀)-beta-aminopropionyl, N-alpha-(R₀)—N-beta-ethylaminopropionyl, N-(R₀)-L-seryl(O—PO₃H₂), N-(R₀)-L-seryl(O—PO₃Me₂), and N-(R₀)-L-threonyl, wherein Ro is lower alkyl or allyl;
or D is a glycosyl derivative of D- or L-serine or D- or L-threonine;
or D is a C₁-C₁₂ ether derivative of D- or L-serine or D- or L-threonine;
or D is a polyethyleneglycol ether derivative of D- or L-serine or D- or L-threonine;
wherein the ethyleneglycol ether portion is selected from 1, 2, 3, 4 or up to 100 (O—C₂H₄—) units; and is preferably selected from 1, 2, 3, or 4 to 50 (O—C₂H₄—) units; or from 1 to 20 (O—C₂H₄—) units; or from 2 to 20 (O—C₂H₄—) units; or from 3 to 20 (O—C₂H₄—) units; or from 4 to 20 (O—C₂H₄—) units; or from 1 to 10 (O—C₂H₄—) units; or from 2 to 10 (O—C₂H₄—) units; or from 3 to 10 (O—C₂H₄—) units; or from 4 to 10 (O—C₂H₄—) units; and wherein said polyether terminates in a group selected from the group consisting of: alkyl, aryl, —OH, —OR, —O(C(O))R, —O(C(O))NHR, —O(C(O))NRR, —O—C₂H₄—CO₂H, —O—CH₂—CO₂H, —NH₂, —NHR, —NR₂, or —NH(C(O))R, —O(C(O))NHR, or —O(C(O))NRR; wherein R is selected independently from lower alkyl, cycloalkyl, benzyl or aryl;
E is an amino acyl residue selected from the group consisting of L-tyrosyl, L-tyrosyl(O-methyl), L-tyrosyl(O-ethyl), L-tyrosyl(O—PO₃H₂), L-tyrosyl(O—PO₃Me₂), D-alanyl, L-phenylalanyl, N—(R₃₃)-L-tyrosyl, N—(R₃₃)-L-tyrosyl(O-methyl), N—(R₃₃)-L-tyrosyl(O—PO₃H₂), N—(R₃₃)-L-tyrosyl(O—PO₃Me₂), 3-(2-thienyl)alanyl, 3-(3-benzothienyl)alanyl, 3-(1-naphthyl)alanyl, 3-(2-naphthyl)alanyl, N—(R₃₃)-L-phenylalanyl, L-3-(4-chlorophenyl)alanyl, L-3-(4-fluorophenyl)alanyl, L-histidyl, L-3-(cyclohexyl)alanyl, L-3-(4-aminophenyl)alanyl, 1-3-(4 acetylaminophenyl)alanyl, N—(R₃₃)-L-3-(4-aminophenyl)alanyl, N—(R₃₃)-L-3-(4-acetylaminophenyl)alanyl, N—(R₃₃)-L-3-(4-fluorophenyl)alanyl, N—(R₃₃)-L-3-(4-chlorophenyl)alanyl, N—(R₃₃)-L-histidyl, N—(R₃₃)-L-3-(cyclohexyl)alanyl, N—(R₃₃)-3-(2-thienyl)alanyl, N—(R₃₃)-3-(3-benzothienyl)alanyl, N—(R₃₃)-3-(1-naphthyl)alanyl, N—(R₃₃)-3-(2-naphthyl)alanyl, and N—(R₃₃)-L-tyrosyl(O-ethyl), wherein (R₃₃) is methyl, ethyl, 2-fluoroethyl, 2,2-difluoroethyl, 2,2,2-trifluoroethyl, propyl or isopropyl;
or E is
—N(R₃₀)—C((CH₂)nR₁)H—(CO)—
wherein n is 1 to 5; R₃₀ is hydrogen, methyl, ethyl, propyl or isopropyl; and R₁ is amino, alkylamino, cycloalkylamino or alkanoylamino; or R₁ is —N(R₃)—C(O)(CH₂)_{ff}R₆₀ or —NHC(N(R₃)₂)═NR₄ wherein R₃ is hydrogen, lower alkyl or cycloalkyl; R₄ is hydrogen, lower alkyl, cycloalkyl, amino or cyano; ff is 0 to 6; and R₆₀ is loweralkyl, dialkylamino, cycloalkyl, aryl, arylalkyl, heterocyclic, (heterocyclic)alkyl or —NHR₁₂₀ wherein R₁₂₀ is hydrogen, loweralkyl, cycloalkyl, aryl, arylalkyl, heterocyclic, (heterocyclic)alkyl, amino, alkanoylamino or —NHR₆₂ wherein R₆₂ is loweralkyl, cycloalkyl, aryl, arylalkyl, heterocyclic, (heterocyclic)alkyl or —C(O)R₆₃ wherein R₆₃ is loweralkyl, cycloalkyl, aryl, arylalkyl, heterocyclic or (heterocyclic)alkyl;
or R₁ is —C(O)R** wherein R** is hydroxy, alkoxy, amino, phenoxy or -methoxyphenyl;
F is a D-aminoacyl residue derived from any of the naturally-occurring alpha amino acids, or from synthetic, non-natural alpha amino acids including (t-Bu)-D-glycyl-, D-seryl(O-tBu), D-3-(2-naphthyl)alanyl, Nt-benzyl-D-histidyl-, and including but not limited to those listed for group B; or
F is a D-amino acyl residue having the formula:
—N(R₃₄)—C((CH₂)_{z}(CO)R₃₇)H—(CO)—
wherein z is 0 to 3 and R₃₇ is hydroxy, alkoxy, phenoxy, amino or p-methoxyphenyl and R₃₄ is hydrogen, methyl, ethyl, propyl or isopropyl;
or F is a D-lysine residue or D-homolysine residue substituted with a polyethyleneglycol group (PEG) on the distal nitrogen wherein the PEG unit is attached via acylation to give an amide derivative of a structure selected from the group consisting of:
—NH—C((CH₂)y-NH—(CO)(CH₂)x-(O—C₂H₄—)n-OH))H—(CO)—; or
—NH—C((CH₂)y-NH—(CO)(CH₂))x-(O—C₂H₄—)n-OR))H—(CO)—;
wherein y is 4 or 5; x is an integer selected from 1-10 inclusive; n is an integer selected from 1-100 inclusive; preferably selected from 1-50, more preferably selected from 1-20; and R is a group selected from lower alkyl, aryl, heteroaryl, benzyl, acyl, aroyl, or heteroaroyl; or
or F is a D-lysine residue or D-homolysine residue substituted with a polyethyleneglycol group (PEG) on the distal nitrogen wherein the PEG unit is attached via alkylation to give an amine derivative of a structure such as:
—NH—C((CH₂)y-NH—(CH₂)xCH₂—(O—C₂H—)n-OR))H—(CO)—; wherein y is 4 or 5; x is an integer selected from 1-10 inclusive; n is an integer from 1-100 inclusive; preferably selected from 1-50, more preferably selected from 1-20; and R is a group selected from lower alkyl, aryl, heteroaryl, benzyl, acyl, aroyl, or heteroaroyl;
or F is a D-citrullinyl residue, D-ornithinyl residue, D-lysyl residue or D-homolysyl residue; or
is a D-ornithinyl residue, D-lysyl residue or D-homolysyl residue substituted on the distal nitrogen with one or two groups selected independently from the group consisting of: H, lower alkyl, aryl, heteroaryl, cycloalkyl, ureido, guanidinyl, —(C(O))R, —(C(O))NHR, —(C(O))NRR, —C₂H₄—CO₂H, —CH₂—CO₂H, —NH₂, —NHR, —NR₂, —NH(C(O))R, (C(O))NHR, and —(C(O))NRR; and wherein R is selected independently from lower alkyl, cycloalkyl, benzyl or aryl;
or F is a glycosyl derivative of D- or L-serine or D- or L-threonine;
or F is a polyethyleneglycol ether derivative of D- or L-serine or D- or L-threonine; wherein the ethyleneglycol ether portion is selected from the group consisting of from 1-100 (O—C₂H₄—) units; or
alternatively is selected from 1-50 (O—C₂H₄—) units; or
alternatively is selected from 1 to 20 (O—C₂H₄—) units; or
alternatively is selected from 2 to 20 (O—C₂H₄—) units; or from 3 to 20 (O—C₂H₄—) units; or from 4 to 20 (O—C₂H₄—) units; or from 1 to 10 (O—C₂H₄—) units; or from 2 to 10 (O—C₂H₄—) units;
or from 3 to 10 (O—C₂H₄—) units; or from 4 to 10 (O—C₂H₄—) units; and
wherein said polyether unit terminates in a group selected from the group consisting of:
alkyl, aryl, —OH, —OR, —O(C(O))R, —O(C(O))NHR, —O(C(O))NRR, —O—C₂H₄—CO₂H, —O—CH₂—CO₂H, —NH₂, —NHR, —NR₂, —NH(C(O))R, —O(C(O))NHR, and —O(C(O))NRR; and
wherein R is selected independently from lower alkyl, cycloalkyl, benzyl or aryl;
provided that if D is a glycosyl derivative or a polyethyleneglycol ether derivative of a serine or threonine, then F can not be a glycosyl derivative or a polyethyleneglycol ether derivative of a serine or threonine or lysine;
G is an amino acyl residue selected from the group consisting of L-arginyl, L-leucyl, D-leucyl, (t-Bu)-D-glycyl-, (t-Bu)-L-glycyl-, L-isoleucyl, norleucyl, alloisoleucyl, valyl, norvalyl, seryl(O-t-Bu), tyrosyl, tryptyl, 2-aminobutyryl, L-(cyclohexyl)alanyl, phenylalanyl, D-tryptyl, tyrosyl, seryl(O-alkyl), prolyl, pipecolyl, L-(β-nicotinoyl)lysyl, seryl and D-seryl;
H is an amino acyl residue selected from L-prolyl, L-arginyl, L-leucyl, L-(β-nicotinoyl)lysyl; or
H is of the formula:
—N(R₃₀)—C((CH₂)nR₁)H—(CO)—
wherein n is 1 to 5; R₃₀ is hydrogen, methyl, ethyl, propyl or isopropyl; and R₁ is amino, alkylamino, cycloalkylamino or alkanoylamino; or R₁ is —N(R₃)—C(O)(CH₂)_{ff}R₆₀ or —NHC(N(R₃)₂)═NR₄ wherein R₃ is hydrogen, lower alkyl or cycloalkyl; R₄ is hydrogen, lower alkyl, cycloalkyl, amino or cyano; ff is 0 to 6; and R₆₀ is loweralkyl, dialkylamino, cycloalkyl, aryl, arylalkyl, heterocyclic, (heterocyclic)alkyl or —NHR₁₂₀ wherein R₁₂₀ is hydrogen, loweralkyl, cycloalkyl, aryl, arylalkyl, heterocyclic, (heterocyclic)alkyl, amino, alkanoylamino or —NHR₆₂ wherein R₆₂ is loweralkyl, cycloalkyl, aryl, arylalkyl, heterocyclic, (heterocyclic)alkyl or —C(O)R₆₃ wherein R₆₃ is loweralkyl, cycloalkyl, aryl, arylalkyl, heterocyclic or (heterocyclic)alkyl;
I is an NH₂ or NHR group wherein R is selected from lower alkyl such as methyl, ethyl, propyl, hydroxyethyl, fluoroethyl, difluoroethyl, or trifluoroethyl; or
I is imino acyl or aliphatic amino acyl residue selected from the group consisting of L-prolyl, L-pipecolyl, alpha-aza-prolyl, trans-beta-aminocyclopentanecarbonyl, cis-beta-aminocyclopentanecarbonyl, 3-(lower alkyl)-prolyl, N-methyl-L-alanyl, N-methyl-norvalyl, 1-dihydroisoindole-2-L-carbonyl and thiazolidine-5-L-carbonyl; or
I is L-(β-isopropyl)lysyl;
and J is nothing if I is an NH₂ or NHR group; or
J is 1-pyrrolidinyl, 1-piperidinyl, 4-morpholinyl, or an amino acyl residue selected from D-alanylamide, L-alanylamide, glycylamide, sarcosylamide, N—(R₄₀)-D-alanylamide, N—(R₄₀)-L-alanylamide, N—(R₄₀)-beta-L-alanylamide, N—(R₄₀)-beta-D-alanylamide, L-2-aminobutyrylamide, D-2-aminobutyrylamide, N—(R₄₀)-L-2-aminobutyrylamide, N—(R₄₀)-D-2-aminobutyrylamide, L-serylamide, D-serylamide, N—(R₄₀)-L-serylamide, N—(R₄₀)-D-serylamide, N—(R₄₀)-L-norvalylamide, N—(R₄₀)-D-norvalylamide, L-norvalylamide, D-norvalylamide, alpha-aza-glycylamide or alpha-aza-alanylamide, wherein R₄₀ is H, methyl, ethyl, propyl or isopropyl.
